(19) United States
(12) Patent Application Publication
(10) Pub. No.: US 2024/0076347 A1
(43) Pub. Date: Mar. 7, 2024

(54) METHODS FOR PREVENTING FUNGAL INFECTIONS

(71) Applicant: Cidara Therapeutics, Inc., San Diego, CA (US)

(72) Inventors: Kenneth BARTIZAL, La Jolla, CA (US); Paul Daruwala, Del Mar, CA (US); Kevin Forrest, Del Mar, CA (US); Brendan Hannah, San Diego, CA (US); Voon Ong, San Diego, CA (US); Maureen Roden, Severna Park, MD (US); Taylor Sandison, Encinitas, CA (US)

(21) Appl. No.: 18/160,807

(22) Filed: Jan. 27, 2023

Related U.S. Application Data
(63) Continuation of application No. 17/846,629, filed on Jun. 22, 2022, now abandoned.
Application No. 17/846,629 is a continuation of application No. 16/464,493, filed on May 28, 2019, now abandoned.
(60) Provisional application No. 62/578,877, filed on Oct. 30, 2017.
(60) Provisional application No. 62/566,947, filed on Oct. 2, 2017.
(60) Provisional application No. 62/541,474, filed on Aug. 4, 2017.
(60) Provisional application No. 62/531,767, filed on Jul. 12, 2017.
(60) Provisional application No. 62/484,696, filed on Apr. 12, 2017.
(60) Provisional application No. 62/427,507, filed on Nov. 29, 2016.

(51) Int. Cl. C07K 14/705; C12Q 1/18

(52) U.S. Cl. CPC C07K 14/70503; C12Q 1/18; A61K 38/00

(57) ABSTRACT

Provided herein are methods for preventing or reducing the likelihood of a fungal infection or related conditions thereto in a human subject in need thereof. The methods include the administration of one or multiple doses of a pharmaceutical composition including CD101 and any pharmaceutically acceptable excipients, wherein the treatment reduces or eliminates the likelihood of developing a fungal infection.

### BACKGROUND

This invention features methods for the prevention of fungal infections and conditions related thereto.

Fungal infections, such as those caused by Candida, Pneumocystis, and Aspergillus, can be serious and life-threatening infections that represent a significant public health issue, particularly in highly vulnerable populations including the elderly, post-surgical, critically ill, and other hospitalized patients with serous medical conditions. Because of increasing resistance to existing antifungal drugs, there is an urgent need to develop new and more effective antifungal agents to treat or prevent these serious infections. Echinocandins are members of a leading class of antifungal agents for the treatment of fungal infections. These compounds target the cell wall by preventing the production of 1,3-β-D-glucan through inhibition of the catalytic subunit of 1,3-β-D-glucan synthase enzyme complex. The three echinocandins approved by the U.S. Food and Drug Administration for the treatment of fungal infections (caspofungin, anidulafungin, and micafungin) are available only in intravenous formulations. Further, these antifungal agents must be administered daily over multiple days, making it challenging to transition patients to a home setting. Further, failure to comply with this multi-day regimen may contribute to the rise in reports of drug-resistant fungal infections. Thus, there is a need in the art for improved methods of preventing and treating fungal infections.

### SUMMARY OF THE INVENTION

The present invention is directed to methods of reducing the likelihood of a fungal infection in a subject (e.g., an immunocompromised subject) by administering to the subject a pharmaceutical composition that includes CD101 salt, or a neutral form thereof.

The invention features a method of reducing the likelihood of a fungal infection in a subject by administering to the subject a pharmaceutical composition including CD101 salt, or a neutral form thereof, and one or more pharmaceutically acceptable excipients, wherein the pharmaceutical composition is administered in an amount and for a duration sufficient to reduce the likelihood of the fungal infection. In some embodiments, the pharmaceutical composition is administered in two or more doses. In some embodiments, the pharmaceutical composition is administered one or more times per year (e.g., 1, 2, 3, 4, 5, or 6 times per year), one or more times per month (e.g., 1, 2, 3, or 4 times per month), one or more times per week (e.g., 1, 2, 3, 4, 5, 6, or 7 times per week), or one or more times per day (e.g., 1, 2, or 3 times per day). In some embodiments, the pharmaceutical composition is administered on consecutive days (e.g., every day), consecutive weeks (e.g., every week), or consecutive months (e.g., every month). In some embodiments, the pharmaceutical composition is administered on non-consecutive days (e.g., every other day, every 3 days, every 4 days, every 5 days, or every 6 days), weeks (e.g., every other week or every 2 or 3 weeks), or months (e.g., every other month or every 2, 3, 4, 5, 6, 7, 8, 9, 10, or 11 months). In some embodiments, the pharmaceutical composition is administered for a duration of about 1 to 8 weeks (e.g., 1 to 3, 2 to 4, 3 to 5, 4 to 6, 5 to 7, or 6 to 8 weeks). In some embodiments, the pharmaceutical composition is administered for a duration of about 2 to 12 months (e.g., 2 to 4, 3 to 5, 4 to 6, 5 to 7, 6 to 8, 7 to 9, 8 to 10, or 9 to 11 months). In some embodiments, the pharmaceutical composition is administered at any frequency for a duration of 1 month, 2 months, 3 months, 4 months, 5 months, 6 months, 7 months, 8 months, 9 months, 10 months, 11 months, or 12 months. In some embodiments, the pharmaceutical composition is administered at any frequency for a duration of 1 to 5 years (e.g., 1 year, 2 years, 3 years, 4 years, or 5 years), from 6 to 10 years (e.g., 6 years, 7 years, 8 years, 9 years, or 10 years), from 11 to 15 years (e.g., 11 years, 12 years, 13 years, 14 years, or 15 years), from 16 to 20 years (e.g., 16 years, 17 years, 18 years, 19 years, or 20 years), from 21 to 25 years (e.g., 21 years, 22 years, 23 years, 24 years, or 25 years), or from 26 to 30 years (e.g., 26 years, 27 years, 28 years, 29 years or 30 years). In some embodiments, the pharmaceutical composition is administered as a lifetime prophylactic (e.g., administered at any frequency starting from when the subject is identified as at risk of a fungal infection to death).

In some embodiments, the method further includes administering a second antifungal agent selected from the group consisting of glucan synthase inhibitors, ergosterol inhibitors, and pharmaceutically acceptable salts thereof. In some embodiments, the second antifungal agent is selected from the group consisting of CD101, caspofungin, micafungin, anidulafungin, enfumafungin, clindamycin, trimethoprim, sulfamethoxazole, cotrimoxazole, VT-1161, VT-1129, VT-1598, VL-2397, fluconazole, albaconazole, bifonazole, butoconazole, clotrimazole, econazole, efinaconazole, fenticonazole, isavuconazole, isoconazole, itraconazole, ketoconazole, luliconazole, miconazole, omoconazole, oxiconazole, posaconazole, pramiconazole, ravuconazole, sertaconazole, sulconazole, terconazole, tioconazole, flucocytosine, voriconazole, atovaquone, pentamidine, primaquine, pyrimethamine, 67-121-A, 67-121-C, amphotericin B, arenomvcin B, aurenin, aureofungin A, aureotuscin, candidin, chinin, chitin synthesis inhibitors, demethoxyrapamycin, dermostatin A, dermostatin B, DJ-400-B1, DJ-400-B2, elizabethin, eurocidin A, eurocidin B, filipin I, filipin II, filipin III, filipin IV, fungichromin, gannibamycin, hamycin, levorin A2, lienomycin, lucensomycin, mycoheptin, mycoticin A, mycoticin B, natamycin, nystatin A, nystatin A3, partricin A, partricin B, perimycin A, pimaricin, polifungin B, rapamycin, rectilavendomvcin, rimocidin, roflamycoin, tetramycin A, tetramycin B, tetrin A, tetrin B, polygodial, benzoic acid, ciclopirox, tolnaftate, undecylenic acid, flucytosine or 5-fluorocytosine, griseofulvin, haloprogin, and pharmaceutically acceptable salts thereof. In some embodiments, the second antifungal agent is administered intraorally, intravenously, intramuscularly, intradermally, intrarterially, subcutaneously, orally, or by inhalation.

In some embodiments, the subject is administered a single dose of a pharmaceutical composition including CD101 salt, or a neutral form thereof, and one or more pharmaceutically acceptable excipients.

In some embodiments, the pharmaceutical composition is administered intraorally, intravenously, intramuscularly, intradermally, intrarterially, subcutaneously, orally, or by inhalation. In some embodiments, the pharmaceutical composition includes from 50 mg to 1200 mg (e.g., 100±50 mg, 200±50 mg, 300±50 mg, 400±50 mg, 500±50 mg, 600±50 mg, 700±50 mg, 750±50 mg, 800±100 mg, 900±100 mg, 1000±100 mg, or 1100±100 mg) of the CD101 salt, or a neutral form thereof. In some embodiments, the pharmaceutical composition includes from 50 mg to 600 mg (e.g., 100±50 mg, 200±50 mg, 300±50 mg, 400±50 mg, 500±50 mg) of the CD101 salt, or a neutral form thereof. In some embodiments, the pharmaceutical composition includes from 600 mg to 1200 mg of the CD101 salt, or a neutral form thereof (e.g., 100±50 mg, 200±50 mg, 300±50 mg, 400±50 mg, 500±50 mg, 600±50 mg, 700±50 mg, 750±50 mg, 800±100 mg, 900±100 mg, 1000±100 mg, or 1100±100 mg).

In some embodiments, the fungal infection is a Candida infection, an Aspergillus infection, or a Pneumocystis infection. In some embodiments, the Pneumocystis infection is in a patient with HIV. In some embodiments, the Pneumocystis infection is in a patient without HIV. In some embodiments, the fungal infection is caused by two or more fungal species. In some embodiments, the fungal infection is a nosocomial infection.

In some embodiments, the administration of the pharmaceutical composition substantially prevents a fungal infection. In some embodiments, the pharmaceutical composition is an antifungal prophylaxis.

In some embodiments, the subject is immunocompromised.

In some embodiments, the subject has a cancer, an autoimmune disorder, or HIV/AIDS.

In some embodiments, the subject is a patient admitted to a hospital. In some embodiments, the hospital has a high institutional incidence of fungal infections in immunocompromised patients. In some embodiments, the institutional incidence of fungal infections in immunocompromised patients is greater than or equal to 5%.

In some embodiments, the likelihood of a fungal infection in the subject is associated with one or more risk factors. In some embodiments, the one or more risk factors is an immunosuppressive condition, an immunosuppressive treatment, or a combination thereof. In some embodiments, the immunosuppressive treatment is a chemotherapy, a radiation therapy, a corticosteroid treatment, an anti-TNF therapy, an immunosuppressive drug, or a combination thereof. In some embodiments, the immunosuppressive condition is associated with a humoral immune deficiency, T cell deficiency, leukopenia, neutropenia, asplenia, complement deficiency, or a combination thereof. In some embodiments, the one or more risk factors is a diagnostic or therapeutic procedure. In some embodiments, the diagnostic or therapeutic procedure is a biopsy, an endoscopy, a catheterization, an intubation, a ventilation, a surgery, an implantation, a transplantation, or a combination thereof. In some embodiments, the diagnostic or therapeutic procedure is a noninvasive surgery, a minimally invasive surgery, or an invasive surgery. In some embodiments, the diagnostic or therapeutic procedure is a central venous catheterization, a peripheral venous catheterization, or a urinary catheterization. In some embodiments, the diagnostic or therapeutic procedure is an endotracheal intubation. In some embodiments, the diagnostic or therapeutic procedure implants a ventricular assist device. In some embodiments, the diagnostic or therapeutic procedure is a solid organ transplant, a bone marrow transplant, a stem cell transplant, or a combination thereof. In some embodiments, the solid organ transplant is a heart transplant, a lung transplant, a renal transplant, a liver transplant, pancreas transplant, small bowel transplant, skin transplant, or a combination thereof. In some embodiments, the stem cell transplant is a haemopoeitic stem cell transplant. In some embodiments, the one or more risk factors is an injury. In some embodiments, the injury is an injury to the skin or mucous membranes. In some embodiments, the injury is a burn. In some embodiments, the one or more risk factors is the use of total parenteral nutrition (TPN). In some embodiments, the one or more risk factors is an age-related risk factor. In some embodiments, the age-related risk factor is an age greater than or equal to 65 years. In some embodiments, the age-related risk factor is an age less than or equal to 31 days. In some embodiments, the one or more risk factors is an environmental risk factor. In some embodiments, the environmental risk factor is environmental contamination by an airborne fungus.

In some embodiments, the likelihood of a fungal infection in the subject is associated with two or more risk factors.

In some embodiments, the CD101 salt is CD101 acetate.

In another aspect, the invention features a method of preventing or treating a biofilm in a subject. The method includes administering to the subject a pharmaceutical composition comprising CD101 salt, or a neutral form thereof, and one or more pharmaceutically acceptable excipients.

In some embodiments of this aspect, the biofilm in the subject is a Candida biofilm (e.g., Candida albicans biofilm or a Candida auris biofilm). In some embodiments, the biofilm is attached to a mucous membrane of the subject.

In another aspect, the invention features a method of preventing biofilm growth on a catheter or killing a biofilm attached to a catheter comprising submerging the catheter in an aqueous solution comprising CD101 salt, or a neutral form thereof, or running an aqueous solution comprising CD101 salt, or a neutral form thereof, through the lumen of the catheter.

In some embodiments of this aspect, the biofilm on the catheter is a Candida biofilm (e.g., Candida albicans biofilm or a Candida auris biofilm).

In any of the above methods, the CD101 could be substituted with compound 2 in salt or neutral form (described herein) or a compound described in U.S. Pat. No. 9,217,014, incorporated herein by reference. For example, CD101 could be substituted with a compound described in U.S. Pat. No. 9,217,014 selected from the group consisting of compound 6, compound 7, compound 12, compound 15, compound 17, compound 23, compound 24, and pharmaceutically acceptable salts thereof. These compounds can be prepared using methods analogous to those described in U.S. Pat. No. 9,217,014.

### Definitions

For the purpose of the present invention, the following abbreviations and terms are defined below.

As used herein, the term “about” refers to a range of values that is ±10% of specific value. For example, “about 150 mg” includes ±10% of 150 mg, or from 135 mg to 165 mg. Such a range performs the desired function or achieves the desired result. For example, “about” may refer to an amount that is within less than 10% of, within less than 5% of, within less than 1% of, within less than 0.1% of, and within less than 0.01% of the stated amount. Other than in the examples, or where otherwise indicated, all numbers expressing quantities of ingredients or reaction conditions used herein should be understood as modified in all instances by the term “about.”

As used herein, the term “anti-TNF therapy” refers to a therapy that uses small molecule and/or protein drugs to inhibit or prevent tumor necrosis factor (TNF) receptor binding and/or activation by a TNF. TNF signaling is involved in the autoimmune and immune-mediated disorders (e.g., systemic lupus erythematosus, rheumatoid arthritis, Crohn's disease, multiple sclerosis, myasthenia gravis, Sarcoidosis, Behcet's disease). Examples of small molecule and/or protein drugs that target the TNF receptor and/or the TNF include, but are not limited to, infliximab, adalimumab, certolizumab pegol, etanercept, golimumab, xanthine derivatives, and bupropion.

As used herein, the terms “associated with” and “related to” refer to symptoms, conditions, diseases, syndromes, or disorders that may be diagnosed in a subject who has developed or is at risk of developing of fungal infections. For example, the fungal infection may be a causal factor for a related condition, a factor that exacerbates a related condition without necessarily being causal, or a symptom or outcome of a related condition. Further, the fungal infection may occur at any point in time relative to the related condition (e.g., before, concomitant with, or after onset of the related condition).

As used herein, the term “CD101 salt” refers to a salt of the compound of Formula 1. CD101 has a structure (below) in which the tertiary ammonium ion positive charge of CD101 is balanced with a negative counterion (e.g., an acetate) in its salt form. The structure of CD101 is depicted below.

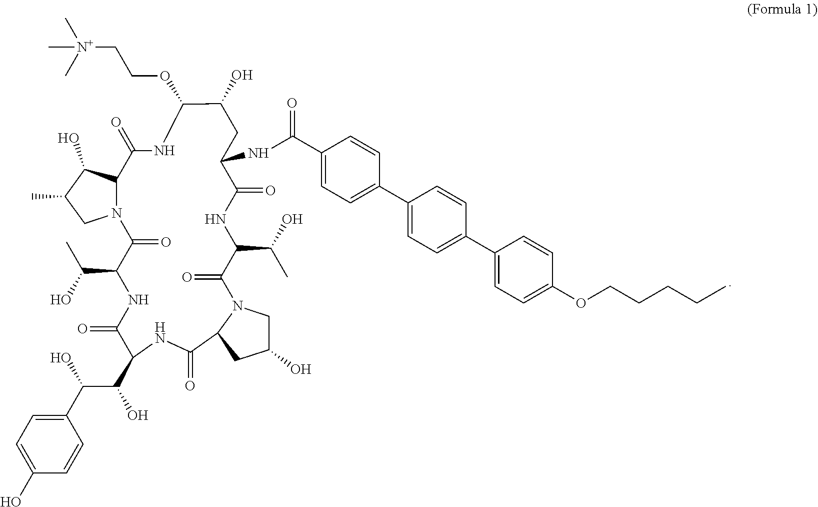

As used herein, the term “compound 2” refers to a salt of the compound of Formula 2, or a neutral form thereof. Compound 2 has a structure (below) in which the tertiary ammonium ion positive charge of the compound in Formula 2 is balanced with a negative counterion (e.g., an acetate) in its salt form. The structure of compound 2 is depicted below.

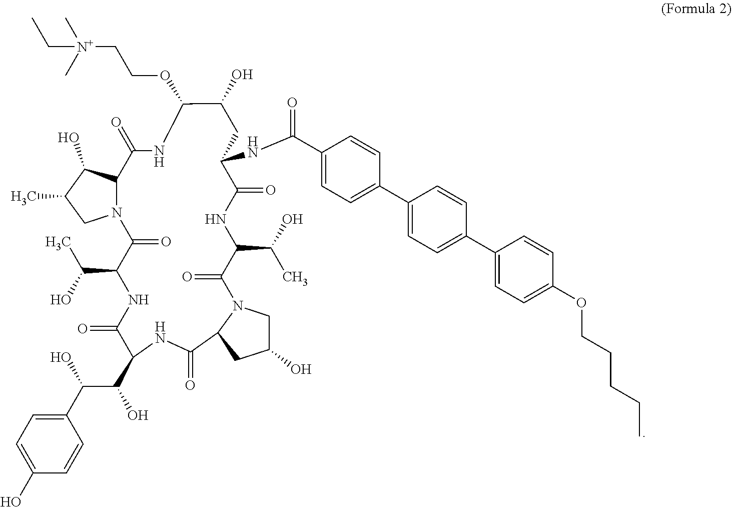

CD101 and compound 2 are semi-synthetic echinocandin compounds that inhibits the synthesis of 1,3-β-D-glucan, an essential component of the fungal cell wall of yeast forms of Candida species and regions of active cell growth of Aspergillus hyphae. The synthesis of 1,3-β-D-glucan is dependent upon the activity of 1,3-β-D-glucan synthase, an enzyme complex in which the catalytic subunit is encoded by FKS1, FKS2, and FKS3 genes. Inhibition of this enzyme results in rapid, concentration-dependent, fungicidal activity for Candida spp. As used herein, the term “CD101 neutral form” includes the zwitterionic forms of CD101 in which the compound of Formula 1 or 2 has no net positive or negative charge. The zwitterion is present in a higher proportion in basic medium (e.g., pH 9) relative to CD101, or a salt thereof. In some embodiments, the zwitterion may also be present in its salt form.

As used herein, the term “CD101 neutral form” or “compound 2 neutral form” includes the zwitterionic forms of CD101 in which the compound of Formula 1 or 2 has no net positive or negative charge. The zwitterion is present in a higher proportion in basic medium (e.g., pH 9) relative to CD101, compound 2, or a salt thereof. In some embodiments, the zwitterion may also be present in its salt form.

The “colonization” of a host organism includes the non-transitory residence of a fungi in or on any part of the body of a human subject. As used herein, “reducing colonization” of a pathogenic fungi (opportunistic or non-opportunistic) in any microbial niche includes a reduction in the residence time of the pathogen and/or a reduction in the number (or concentration) of the pathogen in the colonized part of the subject's body.

The term “combination” or “combination therapy” includes co-administration of a first agent and a second agent, which for example may be dissolved or intermixed in the same pharmaceutically acceptable carrier, or administration of a first agent, followed by the second agent, or administration of the second agent, followed by the first agent. For example, the combination therapy may include CD101 in combination with a different antifungal agent or antibacterial agent, or CD101 in administered in two different dosage forms (e.g., dosage forms for subcutaneous administration and oral administration).

As used herein, the term “chemotherapy” refers to a cancer treatment that uses one or more anticancer drugs. In cancer treatment, chemotherapy is sometimes used in combination with other therapies, such as radiation and surgery to treat cancer.

By “concurrent antifungal treatment” is meant any additional dose of an antifungal agent (e.g., CD101 or another antifungal agent) administered within 12 hours, 24 hours, 2 days, 3 days, 4 days, 5 days, 6, days, or 1 week before or after administration of the single dose of CD101) that would confer therapeutic benefits (e.g., be systemically active) in the treatment of the targeted fungal infection at the same time that CD101 is at a therapeutically effective concentration in the subject. In some instances, the single dose treatment is not combined with any other antifungal treatment within 1-21 days before or after administration. For example, a single dose of CD101 may be administered (e.g., orally, intravenously, subcutaneously, or intramuscularly) to a subject with a fungal infection and the single dose effectively treats the fungal infection without necessitating additional antifungal treatments before, concurrently, or after the single dose treatment with CD101.

As used herein, the term “corticosteroid therapy” refers to a therapy that uses one or more corticosteroids to treat a variety of diseases and conditions, e.g., immune system-related diseases, inflammatory conditions, and skin diseases. Examples of corticosteroids include, but are not limited to, dexamethasone, prednisone, fludrocortisones, and hydrocortisone.

As used herein, “dysbiosis” or “microbial dysbiosis” refers to a state of the microbiota (e.g., fungi and bacteria) on or in any part of the body of a human subject in which the normal diversity and/or function of the ecological network is disrupted. For example, this disrupted state can be due to a decrease in fungal diversity, an overgrowth of one or more fungal pathogens or pathobionts, or a shift to an ecological microbial network that no longer provides an essential function to the host subject, and therefore no longer promotes health. As used herein, the term “pathobiont” or “opportunistic pathogen” refers to symbiotic fungi able to cause disease only when certain genetic and/or environmental conditions are present in a subject.

By “dose” is meant the amount of a compound administered to the human subject.

The term “dosage form” or “unit dosage form”, as used herein, refers to physically discrete units suitable as unitary dosages, such as a pill, tablet, caplet, hard capsule or soft capsule, each unit containing a predetermined quantity of a drug.

By “effective” amount is meant the amount of drug required to treat or prevent a fungal infection or a disease associated with a fungal infection. The effective amount of drug used to practice the methods described herein for therapeutic or prophylactic treatment of conditions caused by or contributed to by a fungal infection varies depending upon the manner of administration, the age, body weight, and general health of the subject. Ultimately, the attending physician will decide the appropriate amount and dosage regimen. Such amount is referred to as an “effective” amount.

As used herein, the term “immunocompromised” or “immunosuppressed” refers to a subject (e.g., a human) who has immune system that functions in an abnormal or incomplete manner, for example, wherein the subject does not have the ability to respond normally to an infection due to what is referred to herein as an “impaired immune system”, “weakened immune system”, or “reduced immune system”. The subject's immune system can be weakened or compromised by a disease (e.g., an HIV infection, an autoimmune disease, cancer), a medical procedure (e.g., an organ transplant (e.g., a solid organ transplant) or a bone marrow transplant), a medical treatment (e.g., an immunosuppressant), and/or a pathogen (e.g., bacteria, fungus, virus). The immune system of the host may also have a congenital defect that renders the host more susceptible to infection. Immunocompromised subjects may be found more frequently among infants, the elderly, and individuals undergoing extensive drug or radiation therapy. Accordingly, aspects of the invention involve the treatment of pediatric and geriatric patients, or patients at risk of a nosocomial infection. Particular patient populations may for example include patients with compromised immune systems due to HIV infection or AIDS, cancer, solid organ transplantation, stem cell transplantation, sickle cell disease or asplenia, congenital immune deficiencies, or chronic inflammatory conditions.

As used herein, the term “immunosuppression therapy” or “immunosuppressive treatment” refers to a therapy that uses one or more immunosuppressants to reduce the activation and/or efficacy of the immune system of a subject (e.g., a human). “Immunosuppressive agents,” “immunosuppressive drugs,” and “immunosuppressants” refer to drugs used in an immunosuppression therapy. In some instances, an immunosuppression therapy is used to prevent the body from rejecting a transplant (e.g., an organ transplant (e.g., a solid organ transplant) or a bone marrow transplant), to treat graft-versus-host disease after a bone marrow transplant, and/or to treat autoimmune diseases (e.g., systemic lupus erythematosus, rheumatoid arthritis, Crohn's disease, multiple sclerosis, myasthenia gravis, Sarcoidosis, Behcet's disease). Immunosuppressants include, but are not limited to, calcineurin inhibitors, mTOR inhibitors, and tyrosine kinase inhibitors (e.g., cyclosporine A, cyclosporine G, voclosporin, tacrolimus, pimecrolimus, sirolimus, temsirolimus, deforolimus, everolimus, zotarolimus, baloniums, ibrutinib, imatinib, dasatinib, nilotinib, erlotinib, sunitinib, gefitinib, bosutinib, neratinib, axitinib, crizotinib, lapatinib, toceranib and vatalanib).

By “infection” or “fungal infection” is meant a microbial dysbiosis characterized by overgrowth or colonization of any part of the body of a human subject by one or more species of fungi (e.g., fungal pathogens or opportunistic pathogens), reduction of which may provide benefit to the host. For example, the infection may include the excessive growth of or colonization by fungal species that are normally present in or on the body of a human subject, or the infection may include colonization by fungal species that are not normally present in or on the body of a human subject. In some instances, the infection may include colonization of a part of the body by a fungus that is indigenous to some parts of the human body (e.g., GI tract) but is detrimental when found in other parts of the body (e.g., tissues beyond the GI tract). More generally, an infection can be any situation in which the presence of a microbial population(s) is damaging to a host body. As used herein, the term “microbiota” refers to the community of microorganisms that occur (sustainably or transiently) in and on the human body.

As used herein, the term “biofilm” refers to a three-dimensional structure composed of heterogeneous fungi (e.g., Candida) and hyphae that can attach to various surfaces, e.g., a mucous membrane or the inside of a catheter. Biofilms can form on the surfaces of medical devices and cause biofilm device-associated infections. For example, having a biofilm on an indwelling device, e.g., a vascular catheter, can cause life-threatening infections.

As used herein, “parenteral administration” and “administered parenterally” refer to modes of administration other than enteral and topical administration, such as injections, and include without limitation intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrastemal injection and infusion.

As used herein, “risk factors” refers to medical conditions (e.g., diseases, disorders, syndromes, or other aberrant biological states), circumstances (e.g., setting or location of a subject or age), and/or events (e.g., exposure, medical procedure, medical treatment, or injury) that increase the likelihood a subject will develop a fungal infection relative to that of an individual who does not experience similar conditions, circumstances, or events. Medical conditions can include a disease or disorder of any etiology (e.g., genetic, acquired, infectious, or idiopathic) that can increase the susceptibility of the subject to a fungal infection. Circumstances can include any situation that the subject experiences (e.g., a long-term stay in a hospital, working in a hospital, or living in a region contaminated with an airborne fungus) that increases the susceptibility of the subject to a fungal infection and/or increases the likelihood of exposure to a fungal pathogen. Events can include any occurrence that happens to a subject (e.g., exposure to a contaminated biological fluid, surgery) that increases the susceptibility of the subject to a fungal infection and/or increases the likelihood of exposure to a fungal pathogen. A subject who is “at risk” or “at high risk” of developing a fungal infection has one or more of these risk factors.

As used herein, the term “salt” refers to any pharmaceutically acceptable salt, such as a non-toxic acid addition salt, metal salt, or metal complex, commonly used in the pharmaceutical industry. Acid addition salts include organic acids, such as acetic, lactic, palmoic, maleic, citric, cholic acid, capric acid, caprylic acid, lauric acid, glutaric, glucuronic, glyceric, glycocolic, glyoxylic, isocitric, isovaleric, lactic, malic, oxalo acetic, oxalosuccinic, propionic, pyruvic, ascorbic, succinic, benzoic, palmitic, suberic, salicylic, tartaric, methanesulfonic, toluenesulfonic, and trifluoroacetic acids, and inorganic acids, such as hydrochloric acid, hydrobromic acid, sulfuric acid, and phosphoric acid. Representative alkali or alkaline earth metal salts include sodium, lithium, potassium, calcium, and magnesium, among others.

As used herein, the term “second antifungal agent” refers to an agent that is used in combination with CD101 to treat or prevent a fungal infection. Second antifungal agents include, but are not limited to, any of the antifungal agents described herein.

As used herein, a “single dose” or “single dose treatment” of CD101 (e.g., CD101 in salt or neutral form) refers to treatment (e.g., substantial elimination) of a fungal infection in a subject by administration of not more than one dose of a pharmaceutical composition including CD101 in salt or neutral form and one or more pharmaceutically acceptable carriers or excipients during a six week, 8 week, or 12 week period. Desirably, the single dose administration is sufficient to treat the fungal infection without requiring a “concurrent antifungal treatment.”

By “subject” or “patient” is meant a human. A human subject who is being treated for a fungal infection is one who has been diagnosed by a medical practitioner as the case may be as having such a condition. Diagnosis may be performed by any suitable means. One in the art will understand that subjects of the invention may have been subjected to standard tests or may have been identified, without examination, as one at high risk due to the presence of one or more risk factors, such as age, genetics, or family history.

As used herein, the term “substantially eliminates” a fungal infection refers to reducing colonization (see definition above) by one or more opportunistic or non-opportunistic pathogenic fungi (e.g., by about 1%, 2%, 5%, 10%, 20%, 30%, 40%, 50%, 60%, 70%, 80%, 90%, or %100 relative to a starting amount) in one or more parts of the body in an amount sufficient to restore a normal fungal population (e.g. approximately the amount found in a healthy individual) and/or allow benefit to the subject (e.g., reducing colonization in an amount sufficient to sustainably resolve symptoms). For example, a fungal infection can be caused by overgrowth of an opportunistic pathogen that is normally present on the human body but has grown above healthy levels, in which case the infection may be eliminated by reducing fungal species to a level typically found in a healthy individual without necessarily eliminating the fungal species. Alternatively, for example, a fungal pathogen or opportunistic pathogen may colonize a portion of the body in which it does not typically reside and thus, the infection is treated when the fungal population is eradicated.

As used herein, the term “substantially prevents” refers to preventing increased colonization by one or more opportunistic or non-opportunistic pathogenic fungi (e.g., by about 1%, 2%, 5%, 10%, 20%, 30%, 40%, 50%, 60%, 70%, 80%, 90%, %100, or more than 100% relative to a starting amount) in one or more parts of the body in an amount sufficient to maintain a normal fungal population (e.g., approximately the amount found in a healthy individual), prevent the onset of a fungal infection, and/or prevent symptoms or conditions associated with infection. For example, subjects may receive prophylaxis treatment to substantially prevent a fungal infection while being prepared for an invasive medical procedure (e.g., preparing for surgery, such as receiving a transplant, stem cell therapy, a graft, a prosthesis, receiving long-term or frequent intravenous catheterization, or receiving treatment in an intensive care unit), in immunocompromised subjects (e.g., subjects with cancer, with HIV/AIDS, or taking immunosuppressive agents), or in subjects undergoing long term antibiotic therapy.

As used herein, the term “treating” refers to administering a pharmaceutical composition for prophylactic purposes. To “prevent” or “reduce the likelihood” of a fungal infection refers to prophylactic treatment of a subject who is not yet ill, but who is susceptible to, or otherwise at risk of, developing a fungal infection. Other features and advantages of the invention will be apparent from the following detailed description, the drawings, and the claims.

### BRIEF DESCRIPTION OF THE DRAWINGS

FIG. 1 is a graph showing kidney fungal burden in neutropenic mice prophylactically treated with a single subcutaneous administration of CD101 and infected with Candida albicans (see Example 1).

FIG. 2A is a graph showing percent survival over time in neutropenic mice prophylactically treated with CD101 and infected with Aspergillus fumigatus (see Example 1).

FIG. 2B is a graph showing the pharmacokinetic profile of CD101 in mice following a 10-mg/kg subcutaneous dose injection.

FIG. 2C is a graph showing a correlation between free drug plasma concentration at time of infection over MIC (0.03 μg/mL) with higher free drug plasma concentration generating greater CFU reduction.

FIG. 3 is a graph showing nuclei counts as a measure of lung fungal burden in mice prophylactically treated with CD101 and infected with Pneumocystis murina (see Example 2).

FIG. 4 is a graph showing asci counts as a measure of lung fungal burden in mice prophylactically treated with CD101 and infected with Pneumocystis murina (see Example 2).

FIG. 5 is a graph showing the percent survival over time after prophylaxis with 3 mg/kg CD101 administered subcutaneously in immune competent DBA/2 female mice infected with A. fumigatus (ATCC 13073) (see Example 3).

FIG. 6 is a graph showing the percent survival over time after prophylaxis with 10 mg/kg CD101 administered subcutaneously in immune competent DBA/2 female mice infected with A. fumigatus (ATCC 13073) (see Example 3).

FIG. 7 is a graph showing the percent survival over time after prophylaxis with 30 mg/kg CD101 administered subcutaneously in immune competent DBA/2 female mice infected with A. fumigatus (ATCC 13073) (see Example 3).

FIG. 8 is a graph showing the kidney fungal burden after prophylaxis with CD101 in immune competent DBA/2 female mice infected with C. albicans (see Example 4).

FIG. 9 is a graph showing percent survival over time in mice infected with Candida auris and treated with 20 mg/kg CD101 (IP), 20 mg/kg fluconazole (PO), or 0.3 mg/kg amphotericin B (IP).

FIGS. 10A and 10B are bar graphs showing the effect of CD101 (0.25 or 1 μg/ml) (FIG. 10A) and fluconazole (1 or 4 μg/ml) (FIG. 10B) on metabolic activity of adhesion phase C. albicans biofilms compared to untreated control.

FIGS. 11A-11E are confocal scanning laser micrographs showing the effect of CD101 and fluconazole on adhesion phase C. albicans biofilms (prevention): top-down three-dimensional view (top panels) and side-views (bottom panels) of biofilms formed by C. albicans treated with: no drug (control; FIG. 11A), 0.25 μg/ml CD101 (FIG. 11B), 1 μg/ml CD101 (FIG. 11C), 1 μg/ml fluconazole (FIG. 11D), and 4 μg/ml fluconazole (FIG. 11E).

FIGS. 11F and 11G are bar graphs showing the thickness of C. albicans biofilms exposed to CD101 (FIG. 11F) and fluconazole (FIG. 11G).

FIGS. 12A and 12B are bar graphs showing the effect of CD101 (0.25 or 1 μg/ml) (FIG. 12A) and fluconazole (1 or 4 μg/ml) (FIG. 12B) on metabolic activity of mature phase C. albicans biofilms compared to untreated control.

FIGS. 13A-13E are confocal scanning laser micrographs showing the effect of CD101 and Fluconazole on mature phase C. albicans biofilms (treatment): Top-down three-dimensional view (top panels) and side-view (bottom panels) of biofilms exposed to: no drug (FIG. 13A), 0.25 μg/ml CD101 (FIG. 13B), 1 μg/ml CD101 (FIG. 13C), 1 μg/ml fluconazole (FIG. 13D), and 4 μg/ml fluconazole (FIG. 13E). Arrows show bulged/broken cells.

FIGS. 13F and 13G are bar graphs showing thickness of C. albicans biofilms exposed to: CD101 (FIG. 13F) and fluconazole (FIG. 13G).

FIGS. 14A-14F are images showing the temporal effect of CD101 (0.25 μg/ml) on formation of C. albicans biofilms. Images were captured immediately from 0 h and followed up to 16 h for biofilms treated with: no drug (FIGS. 14A and 14B), CD101 at low magnification, ×20 (FIGS. 14C and 14D), and CD101 at high magnification, ×63 (FIGS. 14E and 14F). Arrows show bulging, deformed, and broken cells.

FIGS. 15A and 15B are images showing the temporal effect of CD101 (0.25 μg/ml) on 3 h formed C. albicans biofilms. CD101 was added after 3 h biofilm formation and images were captured immediately after adding CD101 (FIG. 15A) and followed up to 16 h (FIG. 15B), magnification, ×63. Arrows show bulging, deformed, and broken cells.

FIG. 16 is a graph showing reductions in kidney colony forming units following CD101 subcutaneous administration.

FIG. 17 is a graph showing plasma levels of CD101 in two cynomolgus monkeys over 10 days after a single 30 mg/kg dose administered subcutaneously.

FIG. 18 is a graph showing total CD101 exposure following intravenous and subcutaneous administration.

FIG. 19 shows an outline of the study design for Example 8.

FIG. 20A is a line graph showing the average group weight of rats with vulvovaginal candidiasis throughout the study. Arrows on x-axis indicate the estradiol treatment days.

FIG. 20B is a line graph showing the average group weights of rat with vulvovaginal candidiasis throughout the study relative to weight on day of infection (Day 0). Arrows on x-axis indicate the estradiol treatment days.

FIG. 21 is a graph showing the pharmacokinetic profile of CD101 in a rat model of vulvovaginal candidiasis (VVC) following a 10 mg/kg intravenous and subcutaneous dose injection.

FIG. 22 is a scatterplot showing vaginal lavage burden Day +1 (24 h) post infection/prior to treatments following localized vaginal infection with C. albicans 529L.

FIG. 23 is a scatterplot showing vaginal lavage burden Day +2 (48 h) post infection.

FIG. 24 is a scatterplot showing vaginal lavage burden Day +3 (72 h) post infection.

FIG. 25 is a scatterplot showing vaginal lavage burden Day +5 (120 h) post infection.

FIG. 26 is a scatterplot showing vaginal lavage burden Day +7 (168 h) post infection.

FIG. 27 is a scatterplot showing vaginal lavage burden Day +9 (216 h) post infection.

FIG. 28A is a bar graph showing the mean daily vaginal lavage burden of the rats in each group over duration of the study following localized vaginal infection with C. albicans 529L and administration with vehicle, CD101, or fluconazole (error bars are geometric standard deviation).

FIG. 28B is a scatterplot showing the daily vaginal lavage burden of each rat over duration of study following localized vaginal infection with C. albicans 529L and administration with vehicle, CD101, or fluconazole.

FIG. 28C is a line graph showing the daily vaginal lavage burden of the rats in each group over duration of study following localized vaginal infection with C. albicans 529L and administration with vehicle, CD101, or fluconazole (error bars are geometric standard deviation).

FIG. 29A is a bar graph showing the geometric mean terminal vaginal tissue burden (vagina, uterus, and uterine horns) Day +9 (216 h) post infection (error bars are geometric standard deviation).

FIG. 29B is a scatterplot showing terminal vaginal tissue burden (vagina, uterus, and uterine horns) Day +9 (216 h) post infection.

FIG. 30 shows animal weights following infection with A. fumigatus strain AF293. The arrows on the x-axis show the immunosuppression day.

FIG. 31 shows a Kaplan Meir plot of survival for a murine model of pulmonary aspergillosis treated with CD101 at 5 mg/kg, 10 mg/kg, or 20 mg/kg zero, one, three, or five days pre-infection or treated with the comparator micafungin at 2 mg/kg zero or one days pre-infection.

FIG. 32 shows unbound CD101 plasma concentrations in healthy human adults relative to antifungal activity.

FIG. 33 shows the tissue distribution of CD101 and associated half-life of CD101 in various organs in rats following a 5 mg/kg IV CD101 dose.

FIG. 34 shows the plasma concentrations of CD101 in mice following single IP doses. Samples were obtained at seven time points over 72 hours. Each symbol represents the mean and standard deviation from three mice. Cmax represents the peak concentration, AUC is from 0 to infinity, and T1/2 the beta elimination half-life.

FIG. 35A shows CD101 dose-response curves against C. albicans. Each symbol represents the mean and standard deviation from three mice. The horizontal dashed-line at 0 represents the burden of organisms in the kidneys of mice at the start of therapy. Data points below the line represent cidal activity and points above the line represent net growth.

FIG. 35B shows CD101 dose-response curves against C. glabrata.

FIG. 35C shows CD101 dose-response curves against C. parapsilosis.

FIG. 36A shows the relationship between total and free drug AUC/MIC and treatment effect for C. albicans. AUC is measured as the total or free AUC over the full treatment course (168 h). Each symbol represents the mean fungal burden from three mice. The horizontal dashed-line at 0 represents the burden of organisms in the kidneys of mice at the start of therapy. Data points below the line represent cidal activity and points above the line represent net growth. The curved line through the data is the best fit line based on the hill equation and the co-efficient of determination (R2) is shown for each organism group.

FIG. 36B shows the relationship between total and free drug AUC/MIC and treatment effect for C. glabrata.

FIG. 36C shows the relationship between total and free drug AUC/MIC and treatment effect for C. parapsilosis.

FIG. 37A shows the relationship between 24 h average free drug AUC/MIC (fAUC/MIC) and treatment effect for C. albicans. Each symbol represents the mean fungal burden from three mice. The horizontal dashed-line at 0 represents the burden of organisms in the kidneys of mice at the start of therapy. Data points below the line represent cidal activity and points above the line represent net growth. The curved line through the data is the best fit line based on the hill equation and co-efficient of determination (R2) is shown for each organism group. Also shown is the maximum effect (Emax), 50% maximum effect (ED50), and slope of the line (N).

FIG. 37B shows the relationship between 24 h average free drug AUC/MIC (fAUC/MIC) and treatment effect for C. glabrata.

FIG. 37C shows the relationship between 24 h average free drug AUC/MIC (fAUC/MIC) and treatment effect for C. parapsilosis.

FIG. 38 shows the efficacy of 5 mg/kg CD101 SC and amphotericin B in the survival of neutropenic ICR female mice infected with A. fumigatus (ATCC 13073). An asterisk (*) indicates a 50 percent or more (≥50%) increase in the survival rate compared to the vehicle control group.

FIG. 39 shows the efficacy of 10 mg/kg CD101 SC and amphotericin B in the survival of neutropenic ICR female mice infected with A. fumigatus (ATCC 13073). An asterisk (*) indicates a 50 percent or more (≥50%) increase in the survival rate compared to the vehicle control group.

FIG. 40 shows the efficacy of 20 mg/kg CD101 SC and amphotericin B in the survival of neutropenic ICR female mice infected with A. fumigatus (ATCC 13073). An asterisk (*) indicates a 50 percent or more (≥50%) increase in the survival rate compared to the vehicle control group.

FIG. 41 is a graph showing CD101 plasma and epithelial lining fluid (ELF) concentration-time profiles following CD101 IP 20 mg/kg administration.

FIG. 42 is a graph showing the survival rate of mice given prophylaxis CD101 IP 20 mg/kg or posaconazole (PO; 2 or 10 mg/kg) as a single dose one day prior to infection in pulmonary aspergillosis.

FIG. 43 is a graph showing CD101 plasma (total- and free-drug) and ELF concentration-time profiles following CD101 IP 20 mg/kg administration.

FIG. 44 is a graph showing the average group weight of mice throughout the study relative to mice weight on Day −4.

FIG. 45 is a graph showing a Kaplan Meier plot of survival for a murine model of pulmonary aspergillosis treated with a single dose of CD101 IP at 20 mg/kg and 60 mg/kg one day pre-infection or treated with the comparator posaconazole 2 mg/kg and 10 mg/kg one day pre-infection.

FIG. 46 is a graph showing the geometric mean terminal lung burden of a murine model of pulmonary aspergillosis treated with a single dose of CD101, micafungin, or posaconazole.

FIG. 47 is a graph showing the geometric mean terminal lung burden of a murine model of pulmonary aspergillosis treated with a single dose of CD101 or micafungin.

### DETAILED DESCRIPTION

Provided herein are methods for preventing or reducing the likelihood of a fungal infection or related conditions thereto in a human subject in need thereof. The methods include the administration of one or multiple doses of a pharmaceutical composition including CD101 and any pharmaceutically acceptable excipients, wherein the treatment reduces or eliminates the likelihood of developing a fungal infection.

### I. Antifungal Prophylaxis

The invention features prophylaxis treatment methods for reducing the likelihood of a fungal infection in a subject in need thereof. In some instances, the methods described herein can be used to prevent a fungal infection associated with a disruption in the levels or composition of fungal species (e.g., Candida infection, Aspergillus infection, or Pneumocystis infection) in or on one or more body regions or tissues of the host subject. Further, the method can be used to prevent symptoms, manifestations, conditions, or diseases associated with a fungal infection. The fungal infection may be associated with one or more fungal species and/or colonization of the fungal species on one or more body regions or tissues of the host subject. Further, the methods provided herein can prevent a fungal infection that originates from any source or origin, including those that originate from a setting within a healthcare institution (e.g., nosocomial infections).

The methods of the invention can reduce the likelihood of a fungal infection, for example, by preventing an increased colonization by one or more opportunistic or non-opportunistic pathogenic fungi (e.g., prevent increase of about 1%, 2%, 5%, 10%, 20%, 30%, 40%, 50%, 60%, 70%, 80%, 90%, %100, or more than %100 relative to a starting amount) in one or more parts of the body in an amount sufficient to maintain a normal fungal population (e.g., approximately the amount found in a healthy individual), prevent the onset of a fungal infection, and/or prevent symptoms or conditions associated with infection.

In some instances, the methods provided herein can be used to reduce the likelihood of a fungal infection associated with, or partially associated with, a fungal infection or fungal overgrowth localized to one or more portions of the human body. In some instances, the fungal species can be any species belonging to the phylum Ascomycota, Basidomycota, Chytridiomycota, Microsporidia, or Zygomycota. The fungal infection or overgrowth can include one or more fungal species, e.g., Candida albicans, C. tropicalis, C. parapsilosis, C. glabrata, C. krusei, C. auris, Saccharomyces cerevisiae, Malassezia globose, M. restricta, or Debaryomyces hansenii, Gibberella moniliformis, Alternana brassicicola, Cryptococcus neoformans, Pneumocystis carinii, P. jirovecii, P. murina, P. oryctolagi, P. wakefieldiae, and Aspergillus clavatus. The fungal species may be considered a pathogen or an opportunistic pathogen. Further, the fungal species may be found indigenously in or on the human body (e.g., occurs sustainably regardless of level or concentration) or it can exist transiently in or on the human body.

In some instances, the methods provided herein can be used to reduce the likelihood of a fungal infection caused by a fungus in the genus Candida (i.e., a Candida infection). For example, a Candida infection can be caused by a fungus in the genus Candida that is selected from the group consisting of Candida albicans, C. glabrata, C. dubliniensis, C. krusei, C. parapsilosis, C. tropicalis, C. orthopsilosis, C. meningoencephalitis, C. guilliermondii, C. rugosa, C. auris, and C. lusitaniae. Examples of Candida infections that can be treated or prevented by the methods of the invention include, but are not limited to candidemia, oropharyngeal candidiasis, esophageal candidiasis, mucosal candidiasis, genital candidiasis, vulvovaginal candidiasis, rectal candidiasis, hepatic candidiasis, renal candidiasis, pulmonary candidiasis, splenic candidiasis, cardiovascular candidiasis (e.g., endocarditis), CNS candidiasis, or invasive candidiasis.

In some instances, the methods provided herein can be used to reduce the likelihood of a fungal infection caused by a fungus in the genus Aspergillus (i.e., an Aspergillus infection). For example, an Aspergillus infection can be caused by a fungus in the genus Aspergillus that is selected from the group consisting of Aspergillus fumigatus, A. flavus, A. terreus, A. niger, A. candidus, A. clavatus, A. lentulus, A. thermomutatus, A. udagawae, A. calidoustus, and A. ochraceus. Examples of Aspergillus infections that can be treated by the methods of the invention include, but are not limited to, aspergillosis (e.g., invasive aspergillosis, central nervous system aspergillosis, or pulmonary aspergillosis). In some instances, a fungal infection may also be a dermatophyte infection, which can be caused by a fungus in the genus Microsporum, Epidermophyton, or Trichophyton.

In some instances, the methods provided herein can reduce the likelihood of a Pneumocystis infection, referring to an infection caused by a fungus in the genus Pneumocystis. Fungi in the genus Pneumocystis include P. carnii, P. jirovecii, P. murina, P. oryctolagi, and P. wakefieldiae. Pneumocystis infections that can be treated by the methods of the invention include, but are not limited to Pneumocystis jirovecii pneumonia (also called Pneumocystis carnii pneumonia, Pneumocysis pneumonia, or PCP).

Further, the methods provided herein can be used to prevent conditions associated with a fungal infection, including, but not limited to, tinea capitis, tinea corporis, tinea pedis, onychomycosis, perionychomycosis, Pityriasis versicolor, oral thrush, vaginal candidosis, respiratory tract candidosis, biliary candidosis, esophageal candidosis, urinary tract candidosis, systemic candidosis, mucocutaneous candidosis, aspergillosis, mucormycosis, paracoccidioidomycosis, North American blastomycosis, histoplasmosis, coccidioidomycosis, sporotrichosis, fungal sinusitis, and chronic sinusitis. Alternatively, the treatment regimens and pharmaceutical compositions described herein can be administered to prevent a blood stream infection or organ infection (e.g., lung, kidney, or liver infections) in a subject.

In some instances, the methods provided herein can be used to reduce the likelihood of a fungal infection by an otherwise drug-resistant fungal infection, which is a fungal infection that is refractory to treatment with an antifungal drug. In such infections, the fungus that causes the infection is resistant to treatment with one or more antifungal drugs (e.g., an antifungal drug-resistant strain of Candida spp.). Antifungal drugs to which the fungus may be resistant include, but are not limited to, azole compounds, echinocandins, polyene compounds, and flucytosine.

### II. Treatment Indications

The methods of treatment provided herein can be used to reduce the likelihood of, or prevent, a fungal infection in a subject, wherein the subject is at high risk of developing a fungal infection or related conditions thereto (e.g., immunocompromised subjects, hospital inpatients, transplant recipients, low birth weight infants, individuals with a genetic susceptibility). In some instances, the methods provided herein can be used to reduce the likelihood of a fungal infection in a subject who has one or more risk factors (e.g., any risk factors described herein), wherein the one or more risk factors indicate an increased likelihood of developing a fungal infection.

As described herein, the methods of the invention can be used to reduce the likelihood of, or prevent, a fungal infection in a subject with one or more risk factors. In some instances, the risk factors can indicate that the subject is at risk of developing a fungal infection. In some instances, the risk factors can indicate the subject's need and/or the subject's likelihood to benefit from prophylaxis or preventative treatments with a pharmaceutical composition including CD101 (e.g., CD101 in salt or neutral form). For example, risk factors that can be used to identify subjects that can benefit from the treatment methods described herein can include medical conditions (e.g., diseases, disorders, syndromes, or other aberrant biological states), circumstances (e.g., setting or location of a subject or age), and/or events (e.g., exposure, medical procedure, medical treatment, or injury) that increase the likelihood a subject will develop a fungal infection relative to that of an individual who does not experience similar conditions, circumstances, or events. In some instances, the one or more risk factors is the use of total parenteral nutrition (TPN).

Subjects may have a higher likelihood of developing a fungal infection due to medical conditions, including diseases or disorders of any etiology (e.g., genetic, acquired, infectious, or idiopathic) that can increase the susceptibility of the subject to a fungal infection (e.g., immunosuppressive condition). Subjects may have a higher likelihood of developing a fungal infection due to circumstances that increase the likelihood of exposure to a fungus and/or lead to a compromised immune system. For example, subjects who may have a higher risk of developing a fungal infection can include subjects who are admitted for a long-term stay in a hospital (e.g., an intensive care unit), subjects who work in a hospital, subjects residing in a region or building contaminated with an airborne fungus, or subjects who are naturally immunocompromised due to age (e.g., premature infants, infants less than 1 month old, or subjects greater than 65 years old). Further, subjects may have a higher likelihood of developing a fungal infection due to particular exposure events that increase the chance of exposure to pathogenic fungal species (e.g., exposure to a contaminated biological fluid, diagnostic or therapeutic procedures, or an environmental contamination). Other events that can occur to a subject include treatment regimens that lead to a reduced immune function in the subject (e.g., immunosuppressive treatment). A subject who is “at risk” or “at high risk” of developing a fungal infection can have one or more (e.g., 1, 2, 3, 4, or 5) of these risk factors.

### Immunocompromised Subjects

The methods provided herein can be used to reduce the likelihood of, or prevent, a fungal infection in a subject who has a reduced immune function (e.g., a subject who is immunocompromised, immunosuppressed, or immune-deficient) and is thus at a high risk of developing a fungal infection. The reduced immune function may arise from any etiology including medical conditions (e.g., immunosuppressive conditions), medical treatment (e.g., immunosuppressive treatments), medical procedures, age, or injuries that reduce the ability of the subject's immune system to prevent fungal infections.

Immunocompromised subjects (e.g., a human) are especially vulnerable to fungal infections. An immunocompromised subject has an immune system that is weakened by a disease or disorder, a medical procedure (e.g., a transplantation procedure or a surgery), a drug (e.g., an immunosuppressive agent), and/or a pathogen (e.g., bacteria, fungus, virus). In some instances, a subject's immune system is compromised by the treatment of any of the diseases or disorders described further herein. In some instances, a subject (e.g., an immunocompromised subject) is about to have, is currently having, or has had a disease or disorder that weakens the immune system. In some instances, a subject (e.g., an immunocompromised subject) is about to undergo or has undergone a transplantation procedure (e.g., an organ transplant (e.g., a solid organ transplant) or a bone marrow transplant) or a radiation therapy. In some instances, a subject (e.g., an immunocompromised subject) is about to be administered, is currently administered, or has been administered one or more drugs that weaken the immune system, e.g., an immunosuppressant (e.g., a corticosteroid). In some instances, a subject (e.g., an immunocompromised subject) is about to undergo, is currently undergoing, or has undergone an immunosuppressive treatment, an anti-TNF therapy, a corticosteroid therapy, a radiation therapy, and/or a chemotherapy. In some instances, a subject's immune system is compromised by a pathogen (e.g., bacteria, fungus, virus), i.e., either the pathogen is currently present within the subject or had previously infected the subject.

In some instances, a subject (e.g., an immunocompromised subject) is HIV positive or has hyper IgM syndrome. In some instances, a subject (e.g., an immunocompromised subject) has a CD4+ T-cell count of less than 200 cells/μl of blood. In some instances, methods described herein may be used to reduce the risk of or to treat a fungal infection in a subject who is HIV positive or has hyper IgM syndrome. In some instances, methods described herein may be used to reduce the risk of or to treat a fungal infection in a subject having a CD4+ T-cell count of less than 200 cells/μl of blood.

Examples of diseases and disorders that weaken a subject's immune system include, but are not limited to, a human immunodeficiency virus (HIV) infection, a hyper IgM syndrome, cancers, autoimmune diseases, neutropenia, CD4 lymphopenia, hematologic disorders, a congenital immune deficiency, Cushing's syndrome, and a nephrotic syndrome. Examples of cancers that weaken a subject's immune system include, but are not limited to, leukemia (e.g., acute lymphoblastic leukemia (ALL), acute myeloid leukemia (AML), chronic myeloid leukemia (CML), small lymphocytic lymphoma (SLL), chronic lymphocytic leukemia (CLL), acute monocytic leukemia (AMOL), and erythroleukemia), Hodgkin's lymphoma, and non-Hodgkin's lymphoma (e.g., lymphoblastic lymphoma, small cell lymphoma (Burkitt's/Non-Burkitt's), and large cell lymphoma). Other cancers that may weaken a subject's immune system include, but are not limited to, bladder cancer, pancreatic cancer, lung cancer, liver cancer, ovarian cancer, colon cancer, stomach cancer, breast cancer, prostate cancer, renal cancer, testicular cancer, thyroid cancer, uterine cancer, rectal cancer, a cancer of the respiratory system, a cancer of the urinary system, oral cavity cancer, skin cancer, larynx cancer, sarcoma, carcinoma, basal cell carcinoma, choriocarcinoma, adenocarcinoma, giant (or oat) cell carcinoma, breast carcinoma, squamous cell carcinoma, multiple myeloma (MM), astrocytoma, oligoastrocytoma, biliary tract cancer, CNS cancer, neuroblastoma, glioblastoma, rhabdomyosarcoma, neuroectodermal cancer, melanoma, inflammatory myofibroblastic tumor and soft tissue tumor. Subjects having a disease or disorder that weaken a subject's immune system can reduce their risk of developing a fungal infection using the methods disclosed herein.

Examples of autoimmune diseases that weaken a subject's immune system include, but are not limited to, alopecia areata, ankylosing spondylitis, antiphospholipid syndrome, Addison's disease, hemolytic anemia, autoimmune hepatitis, hepatitis, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatricial pemphigoid, limited scleroderma (CREST Syndrome), cold agglutinin disease, Crohn's disease, discoid lupus, essential mixed cryoglobulinemia, fibromyalgia-fibromyositis, Graves' disease, Guillain-Barré syndrome, Hashimoto's thyroiditis, hypothyroidism, inflammatory bowel disease, autoimmune lymphoproliferative syndrome (ALPS), idiopathic pulmonary fibrosis, idiopathic thrombocytopenia purpura (ITP), IgA nephropathy, insulin-dependent diabetes, juvenile arthritis, Lichen planus, lupus, Ménière's disease, mixed connective tissue disease, multiple sclerosis, myasthenia gravis, pemphigus vulgaris, pernicious anemia, polyarteritis nodosa, polychondritis, polyglandular syndromes, polymyalgia rheumatica, polymyositis, dermatomyositis, primary agammaglobulinemia, primary biliary cirrhosis, psoriasis, Raynaud's phenomenon, Reiter's syndrome, rheumatic fever, rheumatoid arthritis, sarcoidosis, scleroderma, Sjogren's syndrome, Stiff-man syndrome, systemic lupus erythematosus, Takayasu arteritis, temporal arteritis/giant cell arteritis, ulcerative colitis, uveitis, vasculitis, vitiligo, and Wegener's granulomatosis. Subjects having an autoimmune disease can reduce their risk of developing a fungal infection using the methods disclosed herein.

The methods described herein can be used to reduce the likelihood of, or prevent, a fungal infection in a subject (e.g., a human) having an HIV infection. For example, the methods provided herein can be used to reduce the likelihood of, or prevent, a Pneumocystis fungal infection in a subject with HIV. Pneumocystis jirovecii or PCP is the most common opportunistic infection in subjects with HIV. HIV infects the cells of the immune system by attaching and destroying T-cells (e.g., CD4+ T-cells). An HIV-positive subject with low CD4+ T-cell count is especially susceptible to Pneumocystis infections (e.g., Pneumocystis jirovecii infections). When an HIV-positive subject's T-cell count falls below 200 cells/μl of blood, the subject may be susceptible to other diseases and/or infections, e.g., fungal infections (e.g., Pneumocystis infections). Alternatively, the methods provided herein can be used to reduce the likelihood of, or prevent, a Pneumocystis fungal infection in a subject who does not have HIV.

The methods described herein can be used to reduce the likelihood of, or prevent, a fungal infection in a subject (e.g., a human) having a hyper IgM syndrome. Hyper IgM syndrome refers to a family of genetic disorders in which the level of IgM antibodies is relatively high as a result of a defect in a CD4+ Th2-cell protein (e.g., CD40 ligand). The defect in the CD4+ Th2-cell protein leads to the inability of B-cells to produce antibodies other than IgM. Subjects with hyper IgM syndrome often have a low number of neutrophils and platelets.

The methods described herein can be used to reduce the likelihood of, or prevent, a fungal infection in a subject having neutropenia. Neutropenia refers to a condition characterized by an abnormally low concentration of neutrophils in the blood. Neutrophils make up the majority of circulating while blood cells and serve as the primary defense against microbial infections.

The methods described herein can be used to reduce the likelihood of, or prevent, a fungal infection in a subject (e.g., a human) having a congenital immune deficiency. A congenital immune deficiency refers to a group of immune deficiency disorders present at the time of birth that are caused by genetic defects. Congenital immune deficiencies may occur as a result of defects in B lymphocytes and/or T lymphocytes. Subjects having a congenital immune deficiency are particularly susceptible to infections of the lung, throat, skin, and ear.

The methods described herein can be used to reduce the likelihood of, or prevent, a fungal infection in a subject (e.g., a human) who is immunocompromised due to age. In some instances, immunocompromised subjects may be found more frequently among infants (e.g., subjects ≤1 month, low birth weight infants, or premature infants) or the elderly (e.g., subjects a ≥65 years). Accordingly, aspects of the invention involve the treatment of pediatric and geriatric patients.

The methods described herein can be used to reduce the likelihood of, or prevent, a fungal infection in a subject (e.g., a human) who has undergone or is about to undergo a transplantation procedure (e.g., an organ transplant (e.g., a solid organ transplant) or a bone marrow transplant). In some instances, the subject has undergone or is about to undergo a solid organ transplant (e.g., a heart transplant, a lung transplant, a renal transplant, a liver transplant, pancreas transplant, small bowel transplant, skin transplant, or a combination thereof). In some instances, a subject has undergone or is about to undergo a hematopoietic stem cell transplant. In some instances, a subject who has undergone or is about to undergo a hematopoietic stem cell transplant has a hematologic disorder (e.g., leukemia). In some instances, a subject who has undergone or is about to undergo a transplantation procedure is administered one or more immunosuppressants before, during, and/or after the transplantation procedure to block the adverse effects of organ or tissue rejection by the immune system, or to treat or reduce complications resulting from the transplantation (e.g., graft-versus-host disease (GVHD) and graft rejection).

The methods described herein can be used to reduce the likelihood of, or prevent, a fungal infection in a subject (e.g., a human) who has undergone or is about to undergo a radiation therapy. Certain types of radiation therapy may cause damage to the immune system. For example, radiation to the underarm area, where the lymph nodes are, may cause damage to the lymph nodes and vessels. Radiation directed at pelvic bones may damage the bone marrow within the bones, thus, reducing the production of red and white blood cells.

The methods described herein can be used to reduce the likelihood of, or prevent, a fungal infection in a subject (e.g., a human) who is about to be administered, is currently administered, or has been administered one or more drugs that weaken the immune system, e.g., an immunosuppressant (e.g., a corticosteroid). In some instances, methods described herein may be used to reduce the risk of, or treat, a fungal infection in a subject who is about to undergo, is currently undergoing, or has undergone an immunosuppressive treatment, an anti-TNF therapy, a corticosteroid therapy, a radiation therapy, and/or a chemotherapy. Examples of immunosuppressants used in an immunosuppressive treatment that weaken a subject's immune system include, but are not limited to, calcineurin inhibitors, mTOR inhibitor, and tyrosine kinase inhibitors (e.g., cyclosporine A, cyclosporine G, voclosporin, tacrolimus, pimecrolimus, sirolimus, temsirolimus, deforolimus, everolimus, zotarolimus, biolimus, ibrutinib, imatinib, dasatinib, nilotinib, erlotinib, sunitinib, gefitinib, bosutinib, neratinib, axitinib, crizotinib, lapatinib, toceranib and vatalanib). Examples of small molecule and protein drugs used in an anti-TNF therapy to target the TNF receptor and/or the TNF include, but are not limited to, infliximab, adalimumab, certolizumab pegol, etanercept, golimumab, xanthine derivatives, and bupropion. Examples of corticosteroids in a corticosteroid therapy that weaken a subject's immune system include, but are not limited to, dexamethasone, prednisone, fludrocortisones, and hydrocortisone.

The methods described herein can be used to reduce the likelihood of, or prevent, a fungal infection in a subject (e.g., a human) who has undergone or is about to undergo a diagnostic or therapeutic procedure (e.g., noninvasive procedures, minimally invasive procedures, or invasive procedures) that may increase the risk of exposure to an infectious fungal pathogen and/or weaken the immune system to increase the likelihood of a fungal infection. For example, methods described herein can be used to reduce the likelihood of, or prevent, a fungal infection in a subject (e.g., a human) who has undergone or is about to undergo a biopsy, an endoscopy, a catheterization (e.g., central venous catheterization, peripheral venous catheterization, or a urinary catheterization), an intubation (e.g., an endotracheal intubation), a ventilation, a surgery (e.g., a noninvasive surgery, a minimally invasive surgery, or an invasive surgery), an implantation (e.g., implantation of a ventricular assist device), a transplantation, or a combination thereof.

The methods described herein can be used to reduce the likelihood of, or prevent, a fungal infection in a subject (e.g., a human) having a skin or mucous membrane injury (e.g., a burn). The skin or mucous membrane injury may reduce natural defenses (e.g., skin barrier) and/or weaken the immune system to increase the likelihood of a fungal infection.

In any of the instances of immunosuppression described herein, the immunosuppression can be associated with a humoral immune deficiency, a T cell deficiency, a leukopenia, a neutropenia, an asplenia, a complement deficiency, or a combination thereof. The methods described herein can be used to reduce the likelihood of, or prevent, a fungal infection in a subject (e.g., a human) who is about to develop or has developed any form of immunosuppression, including immunosuppressive conditions associated with a humoral immune deficiency, a T cell deficiency, a leukopenia, a neutropenia, an asplenia, a complement deficiency, or a combination thereof

### Environmental Risk Factors

The methods provided herein can be used to reduce the likelihood of a fungal infection in a subject, where one or more environmental factors increase the risk of developing a fungal infection. In some instances, the subject (e.g., an immunocompromised patient) can be located in an environment (e.g., a healthcare institute (e.g., a hospital or clinic)), wherein there is a high likelihood of being exposed to a pathogenic fungal species. For example, a subject (e.g., an immunocompromised subject) may have an increased risk of developing a fungal infection during a long-term stay (e.g., two or more weeks) in a hospital (e.g., in an intensive care unit). In some instances, the methods of the invention may be used to reduce the likelihood of an infection in a subject (e.g., an immunocompromised patient) admitted to a healthcare institution where there is a high institutional rate of fungal infections (e.g., fungal infections observed in 25% of immunosuppressed patients). Other environmental risk factors that may increase the likelihood of fungal infections include increased exposure to airborne fungi, such as that observed in hospitals undergoing construction or certain regions following a natural disaster

### III. Pharmaceutical Formulations

The invention features methods for treating or preventing a fungal infection or an associated condition thereto by administering a pharmaceutical composition including or consisting of CD101 in salt or neutral form (e.g., by oral, subcutaneous, or intravenous administration). The pharmaceutical composition may be administered to the subject with a pharmaceutically acceptable diluent, carrier, and/or excipient. Depending on the mode of administration and the dosage, the pharmaceutical composition of the methods described herein will be formulated into suitable pharmaceutical compositions to permit facile delivery. The pharmaceutical composition may be in a unit dose form as needed. The amount of active component (e.g., CD101 in salt or neutral form) included in the pharmaceutical composition of the invention are such that a suitable dose within the designated range is provided (e.g., a dose of 50 to 800 mg or 500 mg to 1200 mg of CD101 in salt or neutral form).

A pharmaceutical composition consisting of or including CD101 in salt or neutral form may be formulated for e.g., oral administration, intravenous administration, or subcutaneous administration. In some instances, a pharmaceutical composition consisting of or including CD101 in salt or neutral form may be formulated for oral administration. In some instances, a pharmaceutical composition consisting of or including CD101 in salt or neutral form may be formulated for subcutaneous administration. In some instances, a pharmaceutical composition consisting of or including CD101 in salt or neutral form may be formulated for intravenous administration (e.g., injection or infusion). For injectable formulations, various effective pharmaceutical carriers are known in the art (See, e.g., Remington: The Science and Practice of Pharmacy, 22^{nd} ed., (2012) and ASHP Handbook on Injectable Drugs, 18^{th} ed., (2014)).

Acceptable carriers and excipients in the pharmaceutical composition of the present invention are nontoxic to recipients at the dosages and concentrations employed. Acceptable carriers and excipients may include buffers such as phosphate, citrate, HEPES, and TAE, antioxidants such as ascorbic acid and methionine, preservatives such as hexamethonium chloride, octadecyldimethylbenzyl ammonium chloride, resorcinol, and benzalkonium chloride, proteins such as human serum albumin, gelatin, dextran, and immunoglobulins, hydrophilic polymers such as polyvinylpyrrolidone, amino acids such as glycine, glutamine, histidine, and lysine, and carbohydrates such as glucose, mannose, sucrose, and sorbitol. The compositions may be formulated according to conventional pharmaceutical practice. The concentration of the compound in the formulation will vary depending upon a number of factors, including the dosage of the drug to be administered, and the route of administration.

### Oral Formulations

The pharmaceutical composition of the present invention (e.g., CD101 in salt or neutral form) can be prepared in the form of an oral formulation. Formulations for oral use can include tablets, caplets, capsules, syrups, or oral liquid dosage forms containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like. Formulations for oral use may also be provided in unit dosage form as chewable tablets, non-chewable tablets, caplets, capsules (e.g., as hard gelatin capsules wherein the active ingredient is mixed with an inert solid diluent, or as soft gelatin capsules wherein the active ingredient is mixed with water or an oil medium).

The pharmaceutical compositions of the invention can alternatively be formulated with excipients that improve the oral bioavailability of the compound. For example, the dosage forms of the invention can be formulated for oral administration with medium chain (C8 to C12) fatty acids (or a pharmaceutically acceptable salt thereof), such as capric acid, caprylic acid, lauric acid, or a pharmaceutically acceptable salt thereof, or a mixture thereof. The formulation can optionally include a medium chain (C8 to C12) alkyl alcohol, among other excipients. Alternatively, the compounds of the invention can be formulated for oral administration with one or more medium chain alkyl saccharides (e.g., alkyl (C8 to C14) beta-D-maltosides, alkyl (C8 to C14) beta-D-Gulcosides, octyl beta-D-maltoside, octyl beta-D-maltopyranoside, decyl beta-D-maltoside, tetradecyl beta-D-maltoside, octyl beta-D-glucoside, octyl beta-D-glucopyranoside, decyl beta-D-glucoside, dodecyl beta-D-glucoside, tetradecyl beta-D-glucoside) and/or medium chain sugar esters (e.g., sucrose monocaprate, sucrose monocaprylate, sucrose monolaurate and sucrose monotetradecanoate).

The methods disclosed herein may also further include the administration of an immediate-release, extended release or delayed-release formulation of CD101 in salt or neutral form.

### Parenteral Formulations

The pharmaceutical composition of the present invention (e.g., CD101 in salt or neutral form) may be formulated in the form of liquid solutions or suspensions and administered by a parenteral route (e.g., subcutaneous, intravenous, or intramuscular). The pharmaceutical composition can be formulated for injection or infusion. Pharmaceutical compositions for parenteral administration can be formulated using a sterile solution or any pharmaceutically acceptable liquid as a vehicle. Pharmaceutically acceptable vehicles include, but are not limited to, sterile water, physiological saline, or cell culture media (e.g., Dulbecco's Modified Eagle Medium (DMEM), α-Modified Eagles Medium (α-MEM), F-12 medium). Formulation methods are known in the art, see e.g., Gibson (ed.) Pharmaceutical Preformulation and Formulation (2nd ed.) Taylor & Francis Group, CRC Press (2009).

### IV. Dosage and Administration

A pharmaceutical composition including CD101 (e.g., CD101 in salt or neutral form) may be formulated for, e.g., intraoral administration, intravenous administration, intramuscular administration, intradermal administration, intraarterial administration, subcutaneous administration, oral administration, or administration by inhalation. In some instances, the pharmaceutical composition including CD101 (e.g., CD101 in salt or neutral form) may be formulated for intravenous administration. In some instances, the pharmaceutical composition including CD101 (e.g., CD101 in salt or neutral form) may be formulated for administration by inhalation. In some instances, the pharmaceutical composition including CD101 (e.g., CD101 in salt or neutral form) may be formulated for oral administration. For injectable formulations, various effective pharmaceutical carriers are known in the art. See, e.g., Remington: The Science and Practice of Pharmacy, 22nd ed., (2012) and ASHP Handbook on Injectable Drugs, 18th ed., (2014).

The dosage of the pharmaceutical composition (e.g. CD101 in salt or neutral form) of the present invention depends on factors including the route of administration, the disease to be treated, and physical characteristics, e.g., age, weight, general health, of the subject (e.g., a human). The dosage may be adapted by the physician in accordance with conventional factors such as the extent of the disease and different parameters of the subject. Typically, the amount of the antifungal agent (e.g. CD101 in salt or neutral form) contained within one or more doses may be an amount that effectively reduces the risk of or treats a microbial dysbiosis and associated conditions (e.g. CD101 in salt or neutral form) in a subject without inducing significant toxicity.

In one aspect, the invention features methods for reducing the likelihood, or preventing, a fungal infection or an associated condition thereto by administering a single dose of a pharmaceutical composition including CD101 (e.g., CD101 in salt or neutral form), wherein the single dose is administered in an amount sufficient to prevent the fungal infection without requiring additional doses of an antifungal agent. In some instances, the single dose of CD101 (e.g., CD101 in salt or neutral form) is administered without concurrent administration of an additional antifungal agent (e.g., CD101 or another antifungal agent) within a time period (e.g., within 1 minute, 30 minutes, 1 hour, 2 hours, 12 hours, 24 hours, 2 days, 3 days, 4 days, 5 days, 6 days, or 1 week before or after administration of the single dose of CD101) that would confer therapeutic benefits (e.g., be systemically active) at the same time that CD101 is at a therapeutically effective concentration in the subject. In some instances, the single dose treatment is not combined with any other antifungal treatment within 1-21 days before or after administration. For example, a single dose of CD101 may be administered (e.g., orally, intravenously, subcutaneously, or intramuscularly) to a subject at risk of a fungal infection and the single dose effectively prevents the fungal infection without necessitating additional antifungal treatments before, during, or after the single dose treatment with CD101.

The single dose formulations can be administered to subjects (e.g., humans) in therapeutically effective amounts. In some instances, the single dose can include an oral formulation of CD101 (e.g. CD101 in salt or neutral form), and can be administered in doses of about 50 mg to about 1200 mg (e.g., 75±25 mg, 100±25 mg, 150±50 mg, 200±50 mg, 250±50 mg, 300±50 mg, 350±50 mg, 400±50 mg, 500±100 mg, 600±100 mg, 700±100 mg, 800±100 mg, 900±50 mg, 1000 mg±100 mg, or 1100±100 mg). In other instances, the single dose of CD101 (e.g. CD101 in salt or neutral form) can include a parenteral formulation (e.g., intravenous, subcutaneous, or intramuscular), and can be administered in dosages of about 50-1200 mg (e.g., 75±25 mg, 100±25 mg, 150±50 mg, 200±50 mg, 250±50 mg, 300±50 mg, 350±50 mg, 400±50 mg, 450±50 mg, 500±100 mg, 600±100 mg, 700±100 mg, 800±100 mg, 900±50 mg, 1000 mg±100 mg, or 1100±100 mg).

In another aspect, the invention is directed to a multi-dose prophylactic treatment regiments to prevent fungal infections in a subject (e.g., administering a pharmaceutical composition including CD101 (e.g., CD101 in salt or neutral form)) in combination with a second antifungal agent. The multi-dose treatment regimen may include administration of a pharmaceutical composition including CD101 (e.g., CD101 in salt or neutral form) in combination with any antifungal agent to prevent a fungal infection or related conditions thereto. Antifungal agents include, but are not limited to, any of the agents described herein including different dosage forms of CD101.

In some instances, the multi-dose treatment regimen can include an oral formulation of CD101 (e.g. CD101 in salt or neutral form) or a pharmaceutically acceptable salt thereof, and can be administered in doses of about 2 mg to about 2 g (e.g., about 2 mg to about 2 g, about 2 mg to about 50 mg, about 50 mg to about 150 mg, about 150 mg to about 250 mg, about 250 mg to about 350 mg, 350 mg to 450 mg, 450 mg to 550 mg, 550 mg to 650 mg, 650 mg to 750 mg, 750 mg to 850 mg, 850 mg to 950 mg, 950 mg to 1050 mg, 1g to 1.5 g, or 1.5 g to 2 g). The oral dosage of CD101 (e.g., CD101 in salt or neutral form) or any formulation of an antifungal agent can be administered daily or one or more times per week (e.g., 1, 2, 3, 4, 5, or 6 days a week). For example, the dosage can be administered one or more times per week over the course of 2 to 8 weeks (e.g., 2 to 8 weeks, 2 to 7 weeks, 2 to 6 weeks, 2 to 5 weeks, 2 to 4 weeks, or 2 to 3 weeks).

In some instances, other dosage forms of the multi-dose regimen can include a parenteral (e.g., intravenous or subcutaneous) formulation of CD101 (e.g., CD101 in salt or neutral form). For example, CD101 (e.g., CD101 in salt or neutral form) can be parenterally (e.g., intravenously, intramuscularly, or subcutaneously) administered in dosages of about 50-400 mg (e.g., 50-125 mg, 75-150 mg, 100-175 mg, 125-200 mg, 150-225 mg, 175-250 mg, 200-275 mg, 225-300 mg, 250-325 mg, 275-350 mg, 300-375 mg, 325-400 mg, 50-250 mg, 250-400 mg, or 50-400 mg). In some instances, the first dose contains about 400 mg of CD101, or a neutral form thereof and each of the subsequent doses contains about 200 mg of a salt of CD101, or a neutral form thereof. In some instances, the first dose includes about 400 mg of CD101, or a salt or neutral form thereof, and each of the subsequent doses include about 50-400 mg (e.g., 50-125 mg, 75-150 mg, 100-175 mg, 125-200 mg, 150-225 mg, 175-250 mg, 200-275 mg, 225-300 mg, 250-325 mg, 275-350 mg, 300-375 mg, 325-400 mg, 50-250 mg, 250-400 mg, or 50-400 mg) of CD101, or a salt or neutral form thereof. The parenteral dosage form can be administered once or multiple times at regular or irregular intervals. The administration of the parenteral dosage form can coincide or occur at different times (e.g. 15 minutes, 45 minutes, 1 hour, 12 hours, 24 hours, or 3 days) relative to the time of administration of the oral dosage form. In instances, the parenteral dosage form can be administered to a subject in one or more weekly doses (e.g., 1, 2, 3, or 4 doses/month) or one or more monthly doses (1, 2, 3, 4, 5, 6, 7, 8, 9, 10, 11, 12 doses/year). Alternatively, the parenteral dosage form can be administered once to a subject, with no additional dosages later on.

In a multi-dose treatment regimen, the timing of the administration of a compound of each dosage form (e.g., CD101 in salt or neutral form thereof) depends on the medical and health status of the subject (e.g., a human). The timing of the administration of each dosage form (e.g., CD101 in salt or neutral form) may be optimized by a physician to reduce the likelihood of a fungal infection in a subject.

### Dosage Timing

In any of the methods described herein, the pharmaceutical composition can be administered in any dosage pattern, frequency, or duration to effectively reduce the likelihood of a fungal infection in an individual. In some instances, the pharmaceutical composition is administered one or more times per year (e.g., 1, 2, 3, 4, 5, or 6 times per year), one or more times per month (e.g., 1, 2, 3, or 4 times per month), one or more times per week (e.g., 1, 2, 3, 4, 5, 6, or 7 times per week), or one or more times per day (e.g., 1, 2, or 3 times per day). In some instances, the pharmaceutical composition is administered on consecutive days (e.g., every day), consecutive weeks (e.g., every week), or consecutive months (e.g., every month). In some instances, the pharmaceutical composition is administered on non-consecutive days (e.g., every other day, every 3 days, every 4 days, every 5 days, or every 6 days), weeks (e.g., every other week or every 2 or 3 weeks), or months (e.g., every other month or every 2, 3, 4, 5, 6, 7, 8, 9, 10, or 11 months). In some instances, the pharmaceutical composition is administered for a duration of about 1 to 8 weeks (e.g., 1 to 3, 2 to 4, 3 to 5, 4 to 6, 5 to 7, or 6 to 8 weeks). In some instances, the pharmaceutical composition is administered for a duration of about 2 to 12 months (e.g., 2 to 4, 3 to 5, 4 to 6, 5 to 7, 6 to 8, 7 to 9, 8 to 10, 9 to 11, or 10 to 12 months). In some instances, the pharmaceutical composition is administered at any frequency for a duration of 1 month, 2 months, 3 months, 4 months, 5 months, 6 months, 7 months, 8 months, 9 months, 10 months, 11 months, or 12 months. In some instances, the pharmaceutical composition is administered at any frequency for a duration of 1 to 5 years (e.g., 1 year, 2 years, 3 years, 4 years, or 5 years), from 6 to 10 years (e.g., 6 years, 7 years, 8 years, 9 years, or 10 years), from 11 to 15 years (e.g., 11 years, 12 years, 13 years, 14 years, or 15 years), from 16 to 20 years (e.g., 16 years, 17 years, 18 years, 19 years, or 20 years), from 21 to 25 years (e.g., 21 years, 22 years, 23 years, 24 years, or 25 years), or from 26 to 30 years (e.g., 26 years, 27 years, 28 years, 29 years or 30 years). In some instances, the pharmaceutical composition is administered as a lifetime prophylactic (e.g., administered at any frequency starting from when the subject is identified as at risk of a fungal infection to death).

In any of the methods described herein, the timing of the administration of the pharmaceutical composition including CD101 (e.g. CD101 in salt or neutral form) depends on the medical and health status of the subject (e.g., a human). In some instances, the subject is about to be immunocompromised by a disease, a medical procedure, a drug, and/or a pathogen. In some instances, the subject is already immunocompromised by a disease, a medical procedure, a drug, and/or a pathogen. For example, a subject who is about to undergo an immunosuppressive treatment may be administered a pharmaceutical composition including CD101 (e.g. CD101 in salt or neutral form) before, during, and/or after receiving the immunosuppressive treatment. The timing of the administration of a compound of the pharmaceutical composition including CD101 (e.g. CD101 in salt or neutral form) may be optimized by a physician to reduce the risk of or to treat a fungal infection in a subject (e.g., an immunocompromised subject).

The subject (e.g., a human) treated using the methods of the disclosure can be immunocompromised. The immune system of an immunocompromised subject may be weakened or compromised by a disease (e.g., an HIV infection, an autoimmune disease, cancer), a medical procedure (e.g., an organ transplant (e.g., a solid organ transplant) or a bone marrow transplant), a drug (e.g., an immunosuppressant), and/or a pathogen (e.g., bacteria, fungus, virus). In some instances, a subject (e.g., an immunocompromised subject) is about to have, is currently having, or has had a disease. In some instances, a subject (e.g., an immunocompromised subject) is about to undergo or has undergone a transplantation procedure (e.g., an organ transplant (e.g., a solid organ transplant) or a bone marrow transplant) or a radiation therapy. In some instances, a subject (e.g., an immunocompromised subject) is about to be administered, is currently administered, or has been administered one or more drugs that weaken the immune system, e.g., an immunosuppressant (e.g., a corticosteroid). In some instances, a subject (e.g., an immunocompromised subject) is about to undergo, is currently undergoing, or has undergone an immunosuppressive treatment, an anti-TNF therapy, a corticosteroid therapy, a radiation therapy, and/or a chemotherapy. In some instances, a subject's immune system is compromised by a pathogen (e.g., bacteria, fungus, virus), i.e., either the pathogen is currently present within the subject or had previously infected the subject. For example, the subject can be HIV positive, have hyper IgM syndrome, or have a CD4+ T-cell count of less than 200 cells/μl of blood.

For the treatment of a subject undergoing an immunosuppression treatment, a pharmaceutical composition including CD101 (e.g., CD101 in salt or neutral form) can be administered concurrently or within a few days (e.g., 1-15 days, e.g., 1, 2, 3, 4, 5, 6, 7, 8, 9, 10, 11, 12, 13, 14, or 15 days (e.g., 7 days)), before and/or after, the subject undergoes an immunosuppression treatment.

For the treatment of a subject undergoing an anti-TNF therapy, a corticosteroid therapy, a radiation therapy, or a chemotherapy a pharmaceutical composition including CD101 (e.g., CD101 in salt or neutral form) can be administered concurrently or within a few days (e.g., 1-15 days, e.g., 1, 2, 3, 4, 5, 6, 7, 8, 9, 10, 11, 12, 13, 14, or 15 days (e.g., 7 days)), before and/or after, the subject undergoes an anti-TNF therapy, a corticosteroid therapy, a radiation therapy, or a chemotherapy.

For the treatment of a subject undergoing a transplantation procedure (e.g., an organ transplant (e.g., a solid organ transplant) or a bone marrow transplant), a pharmaceutical composition including CD101 (e.g., CD101 in salt or neutral form) can be administered concurrently or within a few days (e.g., 1-15 days, e.g., 1, 2, 3, 4, 5, 6, 7, 8, 9, 10, 11, 12, 13, 14, or 15 days (e.g., 7 days)), before and/or after, the subject undergoes a transplantation procedure (e.g., an organ transplant (e.g., a solid organ transplant) or a bone marrow transplant). In some instances, the treatment may start before and continue after the organ transplant.

### Antifungal Agents

The methods disclosed herein may be used alone or in a multi-dose treatment regimen to reduce the likelihood, or prevent, fungal infections in a subject at risk thereof. In the multi-dose regimens described herein, a second antifungal agent can be used in combination with the pharmaceutical composition including CD101 (e.g., CD101 in salt or neutral form) to reduce the likelihood of, or prevent, a fungal infection. The second antifungal agent and the pharmaceutical composition including CD101 (e.g., CD101 in salt or neutral form) can be administered concurrently. Alternatively, the pharmaceutical composition including CD101 (e.g., CD101 in salt or neutral form) can be administered first, followed by administration of the second antifungal agent. In some instances, the second antifungal agent is administered first, followed by administration of the pharmaceutical composition including CD101 (e.g., CD101 in salt or neutral form).

Antifungal agents that can be used as a second antifungal agent in combination with a pharmaceutical composition including CD101 (e.g., CD101 in salt or neutral form) include, but are not limited to, CD101 (e.g., a salt of CD101 or neutral form thereof), clindamycin (sold under the brand names CLEOCIN® and DALACIN®), trimethoprim (sold under the brand names PROLOPRIM®, MONOTRIM®, and TRIPRIM®), sulfamethoxazole (sold under the brand name GANTANOL®), cotrimoxazole (a combination of trimethoprim and sulfamethoxazole (aka TMP-SMX); this combination is sold under the brand names BACTRIM®, COTRIM®, SULFATRIM®, and SEPTRA®), atovaquone (sold under the brand name MEPRON®), pentamidine (sold under the brand names NEBUPENT® and PENTRAM®), primaquine, pyrimethamine (sold under the brand name DARAPRIM®), and pharmaceutically acceptable salts thereof.

Alternatively, the second antifungal agent described herein can be selected from glucan synthase inhibitors (e.g., echinocandins, enfumafungins), polyene compounds, azole compounds, and pharmaceutically acceptable salts thereof.

Glucan synthase inhibitors that can be used as a second antifungal agent include, but are not limited to echinocandins (e.g., caspofungin, micafungin, or anidulafungin) enfumafungin (e.g., SCY-078 (aka MK-3118, see Lepak et al., Antimicrobial agents and chemotherapy 59:1265 (2015)), and pharmaceutically acceptable salts thereof.

The azole compounds are antifungal compounds that contain an azole group (i.e., a five-membered heterocyclic ring having at least one N and one or more heteroatoms selected from N, O, or S). Azole compounds function by binding to the enzyme 14α-demethylase and disrupt, inhibit, and/or prevent its natural function. The enzyme 14α-demethylase is a cytochrome P450 enzyme that catalyzes the removal of the C-14 α-methyl group from lanosterol before lanosterol is converted to ergosterol, an essential component in the fungal cell wall. Therefore, by inhibiting 14α-demethylase, the synthesis of ergosterol is inhibited. Azole compounds that can be used in the first dosage form of the invention include, but are not limited to (e.g., VT-1161, VT-1129, VT-1598, fluconazole, albaconazole, bifonazole, butoconazole, clotrimazole, econazole, efinaconazole, fenticonazole, isavuconazole, isoconazole, itraconazole, ketoconazole, luliconazole, miconazole, omoconazole, oxiconazole, posaconazole, pramiconazole, ravuconazole, sertaconazole, sulconazole, terconazole, tioconazole, and voriconazole), VL-2397, and flucytosine (ANCOBON®).

Polyene compounds are compounds that insert into fungal membranes, bind to ergosterol and structurally related sterols in the fungal membrane, and disrupt membrane structure integrity, thus causing leakage of cellular components from a fungus that causes infection. Polyene compounds typically include large lactone rings with three to eight conjugated carbon-carbon double bonds and may also contain a sugar moiety and an aromatic moiety. Polyene compounds typically include large lactone rings with three to eight conjugated carbon-carbon double bonds and may also contain a sugar moiety and an aromatic moiety. Polyene compounds that can be used in the first dosage form of the invention include, but are not limited to, 67-121-A, 67-121-C, amphotericin B, derivatives of amphotericin B (e.g., C35deOAmB; see Gray et al., Proceedings of the National Academy of Sciences 109:2234 (2012)), arenomvcin B, aurenin, aureofungin A, aureotuscin, candidin, chinin, chitin synthesis inhibitors (e.g., lufenuron), demethoxyrapamycin, dermostatin A, dermostatin B, DJ-400-B1, DJ-400-B2, elizabethin, eurocidin A, eurocidin B, filipin I, filipin II, filipin III, filipin IV, fungichromin, gannibamycin, hamycin, levorin A2, lienomycin, lucensomycin, mycoheptin, mycoticin A, mycoticin B, natamycin, nystatin A, nystatin A3, partricin A, partricin B, perimycin A, pimaricin, polifungin B, rapamycin, rectilavendomvcin, rimocidin, roflamycoin, tetramycin A, tetramycin B, tetrin A, tetrin B, and pharmaceutically acceptable salts thereof.

Other compounds that have antifungal properties that may be used as the second antifungal agent include, but are not limited to polygodial, benzoic acid, ciclopirox, tolnaftate, undecylenic acid, flucytosine or 5-fluorocytosine, griseofulvin, and haloprogin.

### EXAMPLES

### Example 1. Prophylactic, Single-Dose, Subcutaneous Administration of CD101 Shows Robust Efficacy in Neutropenic Mouse Models of Candidiasis and Aspergillosis

The potential for intermittent subcutaneous (SC) administration of CD101 may extend the utility of CD101 beyond that of other echinocandins, to include antifungal treatment and prophylaxis in the outpatient setting. Neutropenic mouse models of candidiasis and aspergillosis were used to evaluate the in vivo efficacy of single SC doses of CD101 as antifungal prophylaxis.

### Method

Candidiasis model: ICR mice (5/group) were rendered neutropenic by cyclophosphamide on Day −4 (150 mg/kg) and Day −1 (100 mg/kg), then challenged (Day 0) with Candida albicans ATCC SC5314 via IV (100 μL, 105 CFU/mouse). Prior to challenge, mice were given one SC dose (5, 10, or 20 mg/kg) of CD101 on Day −5, Day −3, or Day −1. At 24 hours post-challenge, kidneys were removed for CFU enumeration.

Aspergillosis model: ICR mice (6/group) were rendered neutropenic by cyclophosphamide on Day −3 (6 mg/mouse), Day +1, and Day +4 (2 mg/mouse). Challenge with Aspergillus fumigatus ATCC via IV (100 μL, 104 CFU/mouse) occurred on Day 0. Prior to challenge, mice were given one SC dose (5, 10 or 20 mg/kg) of CD101 on Day −5, Day −3, or Day −1. Survival was monitored for 14 days.

### Results

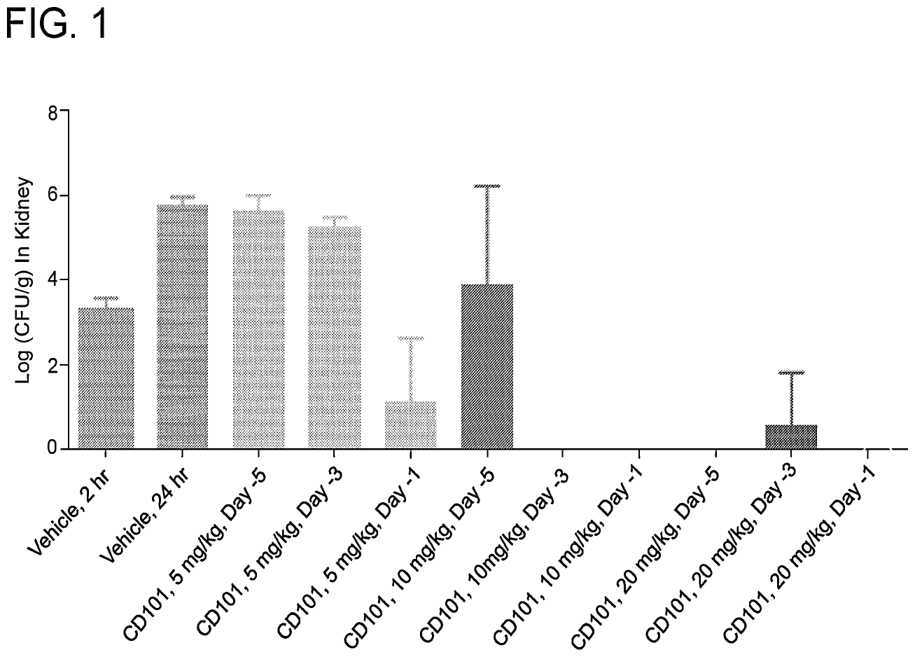

In the candidiasis model (FIG. 1), kidney CFU decreased with increasing doses of CD101 and prophylaxis occurring closer to challenge. Complete clearance was observed in all animals receiving 10 mg/kg at Day −3 and Day −1 and all but one animal receiving 20 mg/kg on Day −3. At doses of 5 or 10 mg/kg, prophylaxis with CD101 demonstrated a significant decrease in CFU at Day −3 and Day −1. At the highest dose of 20 mg/kg, CD101 reduced CFU burden regardless of prophylactic treatment day.

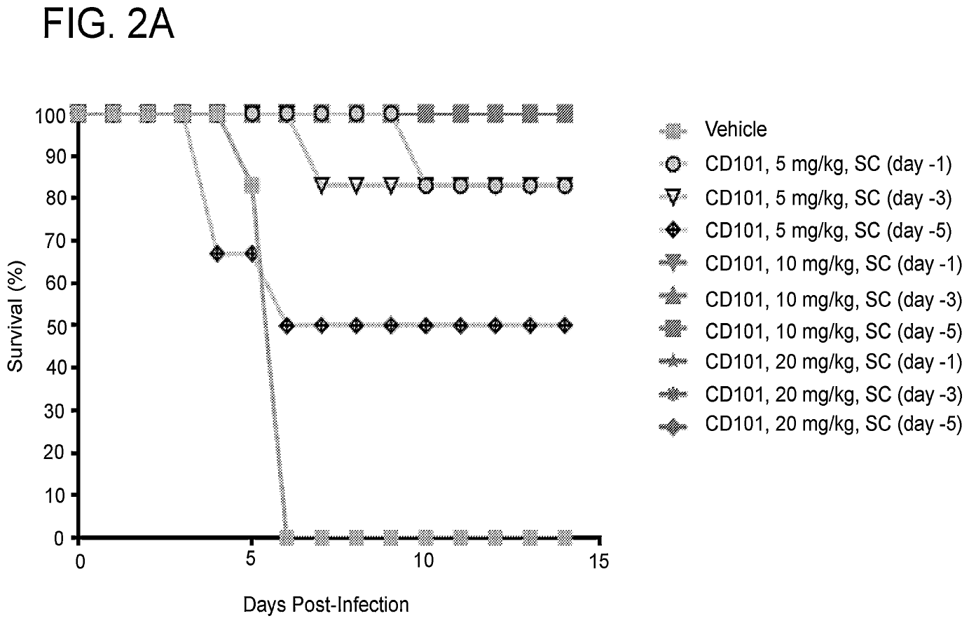

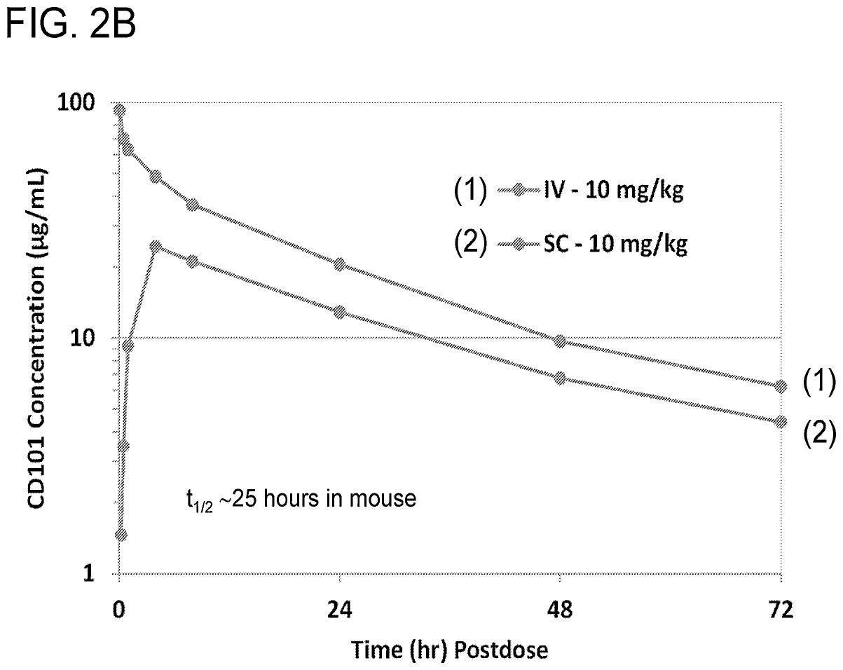

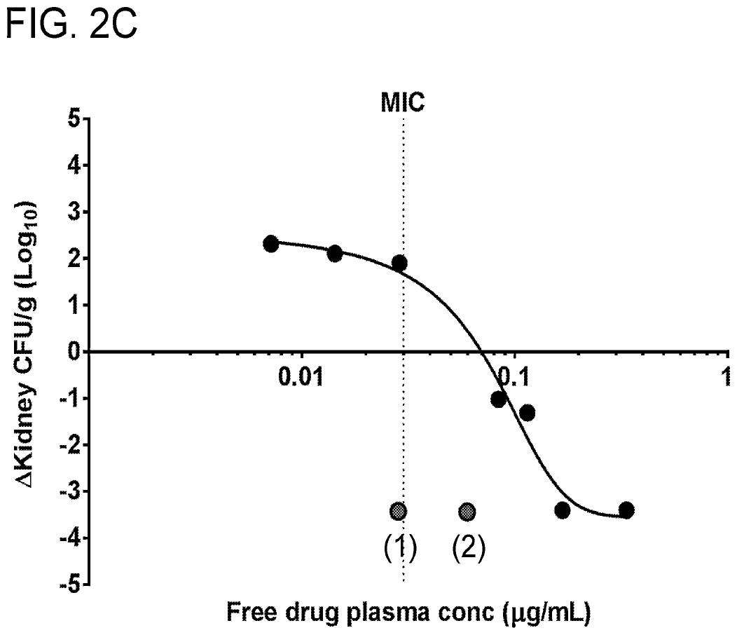

In the aspergillosis model (FIG. 2A), survival was monitored for 14 days after challenge. Subcutaneous CD101 at 5, 10, and 20 mg/kg on Day −5, Day −3 or Day −1 were associated with significant (>50%) increases in survival compared with vehicle. The 5 mg/kg group showed increased survival when prophylaxis was given closer to challenge. All animals in the 10 and 20 mg/kg groups survived regardless of prophylactic treatment day.

The pharmacokinetic profile of CD101 in mice following a 10-mg/kg subcutaneous dose shows a half-life of ˜25 hrs with an absolute bioavailability of ˜50% (FIG. 2B). The AUC from subcutaneous 10 mg/kg in mouse approximates an IV 200 mg dose in human. In general, a correlation was noted between free drug plasma concentration at time of infection over MIC (0.03 μg/mL) with higher free drug plasma concentration generating greater CFU reduction as shown in FIG. 2C for the candidiasis model. Exceptions as indicated by (1) and (2) in FIG. 2C correspond to 10 and 20 mg/kg (Day −3 and −5 respectively) and indicate an apparent hysteresis where effective prophylaxis occurs despite low plasma concentration likely due to slower clearance from tissues.

### Example 2. In Vivo Anti-Pneumocystis Murina Mouse Prophylaxis Study of CD101

### Method

C3H/HeN mice (Charles River) were infected with P. murina by intranasal inoculation of P. murina organisms at 2×10⁶/50 μl from a liquid nitrogen repository. Prior to inoculation, the P. murina were pre-incubated overnight in RPMI 1640 medium supplemented with calf serum and antibiotics to eliminate any bacterial contamination. The immune systems of the mice were suppressed by the addition of dexamethasone at 4 μg/mliter to acidified drinking water (sulfuric acid at 1 ml/liter). Acidification is used to prevent secondary microbial infections. The mice were divided into a negative control group (control steroid—C/S), positive control group (trimethoprim/sulfamethoxazole—TMP/SMX) and treatment groups. Drugs to be tested were administered intraperitoneally (IP) on a mg/kg/d basis; dose, route, and frequency of administration varied depending on the agent being tested.

CD101 was administered at the same time the mice were inoculated. Immune suppression and treatment continued for the entire 6 week study. At that time, the mice were euthanized by CO₂ and lungs processed for analysis by homogenization. Slides were made from the lung homogenates at different dilutions and stained with Diff-Quik to quantify the trophic forms and cresyl echt violet to quantify the asci.

TABLE 1
CD101 Prophylaxis Study Design

| Group | Drug | Dose | | # of Animals |
|---|---|---|---|---|
| 1 | Negative Control | — | | 10 |
| 2 | CD101 | 20 | mg/kg/3×/wk | 10 |
| 3 | ″ | 20 | mg/kg/1×/wk | 10 |
| 4 | CD101 | 2 | mg/kg/3×/wk | 10 |
| 5 | ″ | 2 | mg/kg/1×/wk | 10 |
| 6 | CD101 | 0.2 | mg/kg/3×/wk | 10 |
| 7 | ″ | 0.2 | mg/kg/1×/wk | 10 |
| 8 | TMP/SMX | 50/250 | mg/kg/3×/wk | 10 |

Efficacy is based on the reduction of organism burden between the treatment groups and the negative control group was determined by microscopic evaluation. The nuclei and asci counts for each lung were log transformed and statistical analysis was determined by the analysis of variance (ANOVA); individual groups were compared by the Student-Newman-Keuls t test for multiple comparisons using GraphPad Prism. Statistical significance was accepted at a p value <0.05.

### Results

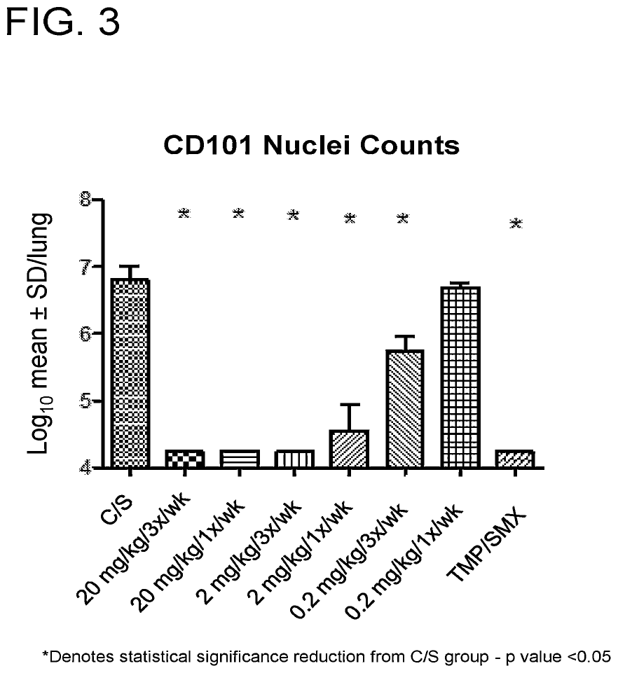

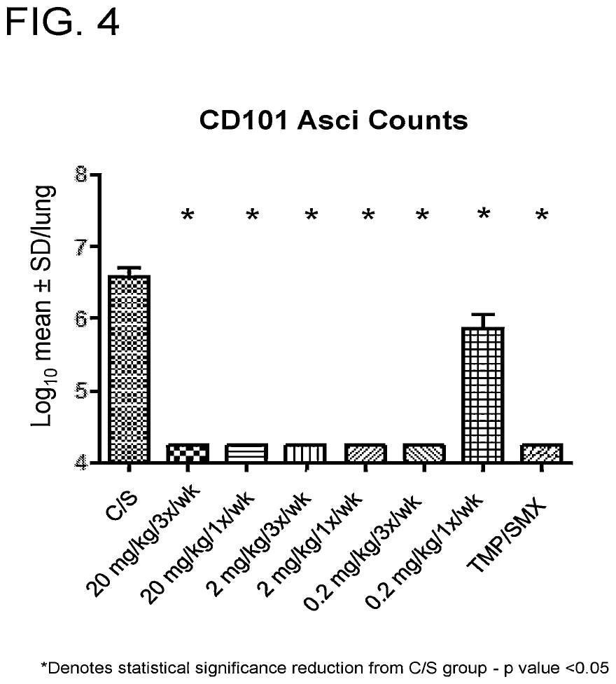

Prophylactic treatment of P. murina infected mice with CD101 showed a statistically significant reduction in nuclei levels at all dose levels except for the 0.2 mg/kg/1×/week group as compared to the negative control group after 6 weeks of dosing (FIG. 3). Three of the treatment groups were equally as efficacious as the positive control TMP/SMX with no nuclei seen by microscopic evaluation. All CD101 treatment groups showed a statistically significant reduction in asci levels compared to the negative control group and there was no difference in efficacy between 5 treatment groups and the positive control TMP/SMX with no asci observed by microscopic evaluation (FIG. 4).

### Example 3. A. fumigatus (ATCC 13073) Disseminated Infection of Immune Competent Mice: CD101 Prophylactic Efficacy

### Summary

The study objective was to evaluate the efficacy of the test article, CD101, as prophylaxis in the Aspergillus fumigatus (ATCC 13073) disseminated infection model with immune competent DBA/2 mice.

### Method

A. fumigatus (ATCC 13073) growth was taken from 96 hour potato dextrose agar (PDA) and re-suspended in 0.1% Tween 20. The culture was resuspended in 1 mL cold PBS (>1.0×10⁸ CFU/mL, OD₆₂₀ 2.3-2.8). The culture was then diluted in PBS to final cellular densities of 1.0×10⁷ CFU/mL. The actual colony counts were determined by plating dilutions on PDA plates to confirm inoculation concentration. The actual inoculum count was 1.30×10⁷ CFU/mL.

TABLE 2
Study Design

| | Test | | | Dosing | Conc. | Conc. | Dosage | | Mice |
|---|---|---|---|---|---|---|---|---|---|
| Grp | Article | Route | Schedule | Day | mg/mL | mL/kg | mg/kg | mg/kg | (ICR) |
| 1 | Vehicle | — | None | — | — | — | — | 3 | 6 |
| 2 | AmpB | IP | Single | 0 | 0.3 | 10 | — | 3 | 6 |
| 3 | CD101 | SC | Single | 0 | 0.3 | 10 | 3 | 3 | 6 |
| 4 | CD101 | SC | Single | −5 | 0.3 | 10 | 3 | 3 | 6 |
| 5 | CD101 | SC | Single | −3 | 0.3 | 10 | 3 | 3 | 6 |
| 6 | CD101 | SC | Single | −1 | 0.3 | 10 | 3 | 10 | 6 |
| 7 | CD101 | SC | Single | −5 | 1 | 10 | 3 | 10 | 6 |
| 8 | CD101 | SC | Single | −3 | 1 | 10 | 10 | 10 | 6 |
| 9 | CD101 | SC | Single | −1 | 1 | 10 | 10 | 30 | 6 |
| 10 | CD101 | SC | Single | −5 | 3 | 10 | 10 | 30 | 6 |
| 11 | CD101 | SC | Single | −3 | 3 | 10 | 30 | 30 | 6 |
| 12 | CD101 | SC | Single | −1 | 3 | 10 | 30 | 3 | 6 |

Groups of 6 immune competent female DBA/2 mice weighing 18±2 g were used. On Day 0, animals were inoculated (0.1 mL/mouse) by intravenous (IV) injection into the tail vein with A. fumigatus (ATCC 13073), 1.30×10⁶ CFU per mouse. CD101 at 3, 10 and 30 mg/kg as prophylaxis was administered subcutaneously (SC) once starting 5, 3 or 1 day before inoculation. In addition, CD101 at 3 mg/kg SC and the reference, amphotericin B, at 3 mg/kg by intraperitoneal (IP) injection were administered one hour after infection (See Table 2).

Mortality was observed for 14 days. A 50 percent or more (≥50%) increase in the survival rate compared to the vehicle control group indicates significant anti-infective activity. The health observations including body weight, hunched posture, ruffled fur, immobility and hypothermia were recorded daily for 14 days.

### Results

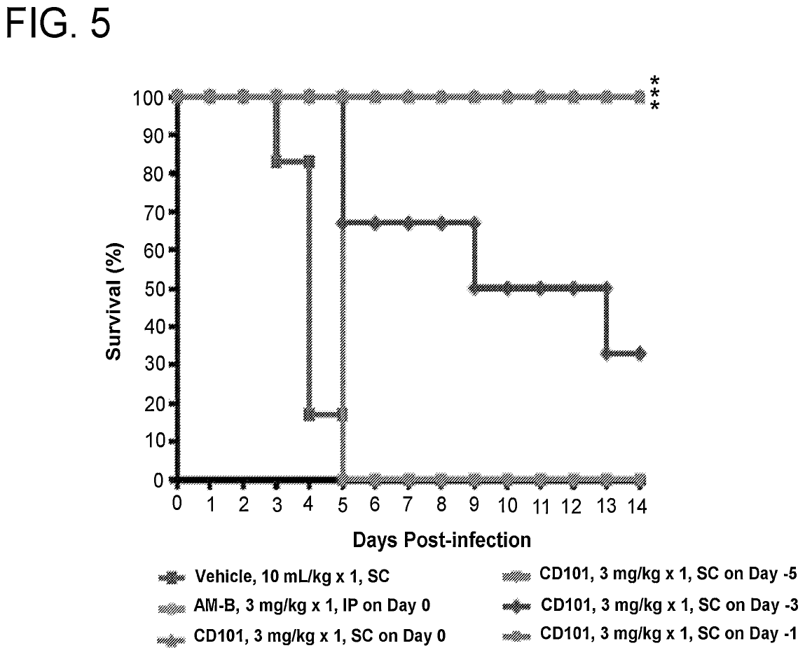

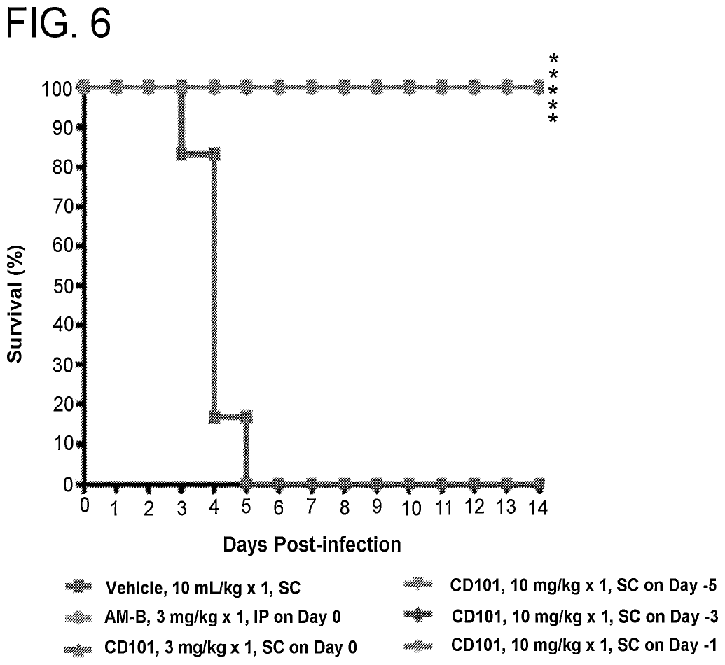

Subcutaneous administrations of CD101 at 3 mg/kg on Day −1, 10 mg/kg on Day −5, Day −3 and Day −1 and 30 mg/kg on Day −5, Day −3 and Day −1 were associated with significant (50%) increase in the 14-day survival compared to the vehicle group (FIGS. 5-7). CD101 at 3 mg/kg SC and amphotericin B at 3 mg/kg IP administered 1 hour after infection were also associated with significant increase in the 14-day survival observation in the study. In addition, the symptoms of infection including a decrease in the body weight, hunched posture, ruffled fur, immobility and hypothermia from were improved by subcutaneous administrations of CD101 at 3, 10 and 30 mg/kg on Day −5, Day −3 and Day −1 before infection.

### Example 4. Kidney Fungal Burden Assessment in Fully Immunocompetent DBA/2 Mice in a Disseminated Candidiasis Model of Infection with Prophylactic Treatment of CD101

### Summary

The purpose of this study was to evaluate CD101 in fully immunocompetent DBA/2 mice after challenge with Candida albicans (ATCC SC5314) via IV injection in the lateral tail vein at 5 log10.

### Method

Cultures of Candida albicans (ATCC SC5314) were grown overnight at 37° C. in an ambient atmosphere on Sabouraud agar. Cultures were removed from the incubator, aseptically transferred to tubes of phosphate buffered saline (PBS), and an optical density was determined at 600 nm. The cultures were diluted to provide a challenge inoculate of approximately 5.0 log₁₀ CFU per mouse in a volume of 100 μL. Each animal was administered an intravenous (IV) challenge in a volume of 100 IL on Day 0. Instillation of the challenge constituted time 0 hour for the study. The final count of the challenge inoculum demonstrated a delivered burden of 5.0 log₁₀ organisms per mouse.

Mice were treated with CD101 one time via subcutaneous (SC) injection prior to challenge on either Day −5, Day −3, Day −1 or Day 0 per Table 3. Micafungin and vehicle controls were administered on Day 0 as per Table 3. Day 0 treatment or control administration occurred immediately following the challenge. Test articles were administered in a volume of 0.5 mL.

TABLE 3
Study Design

| | | Fungal Challenge | | | | | | | |
|---|---|---|---|---|---|---|---|---|---|
| | | (C. albicans | | Treatment | | | | | |
| | | SC5314) | | | Dose | Dosing | | | Kidney |
| Grp | n | Log₁₀ | ROA | Description | mg/kg | Day | | ROA | Harvest |
| 1 | 3 | 5.0 | IV | Vehicle | NA | Day | 0 | SC | 2 hr |
| 2 | 5 | 5.0 | IV | Vehicle | NA | Day | 0 | SC | 24 hr |
| 3 | 5 | 5.0 | IV | Micafungin | 1 | Day | 0 | IP | 24 hr |
| 4 | 5 | 5.0 | IV | CD101 | 1 | Day | 0 | SC | 24 hr |
| 5 | 5 | 5.0 | IV | CD101 | 3 | Day | −5 | SC | 24 hr |
| 6 | 5 | 5.0 | IV | CD101 | 3 | Day | −3 | SC | 24 hr |
| 7 | 5 | 5.0 | IV | CD101 | 3 | Day | −1 | SC | 24 hr |
| 8 | 5 | 5.0 | IV | CD101 | 10 | Day | −5 | SC | 24 hr |
| 9 | 5 | 5.0 | IV | CD101 | 10 | Day | −3 | SC | 24 hr |
| 10 | 5 | 5.0 | IV | CD101 | 10 | Day | −1 | SC | 24 hr |
| 11 | 5 | 5.0 | IV | CD101 | 30 | Day | −5 | SC | 24 hr |
| 12 | 5 | 5.0 | IV | CD101 | 30 | Day | −3 | SC | 24 hr |
| 13 | 5 | 5.0 | IV | CD101 | 30 | Day | −1 | SC | 24 hr |

The primary endpoint used to assess progress of the infection was mean CFU burden per gram of kidney tissue. At 24 hours post-challenge, mice were humanely euthanized via CO₂ overexposure. Kidneys were aseptically removed, weighed, and transferred to sterile tubes containing 2 mL of PBS. Tissues were homogenized with a mini-bead beater. Serial dilutions of the tissue homogenates were plated on Sabouraud dextrose agar plates and incubated at 37° C. for 24-48 hours. CFU/g tissue was determined from colony counts.

### Results

All challenge procedures were performed as detailed in the study protocol. One animal in Group 2 (vehicle control) died before harvest. NOTE: Group 5 animals (CD101, 3 mg/kg) were to be dosed on Day −5 of study and were inadvertently dosed on Day −5 and Day −3 of study, receiving two doses of compound prior to challenge.

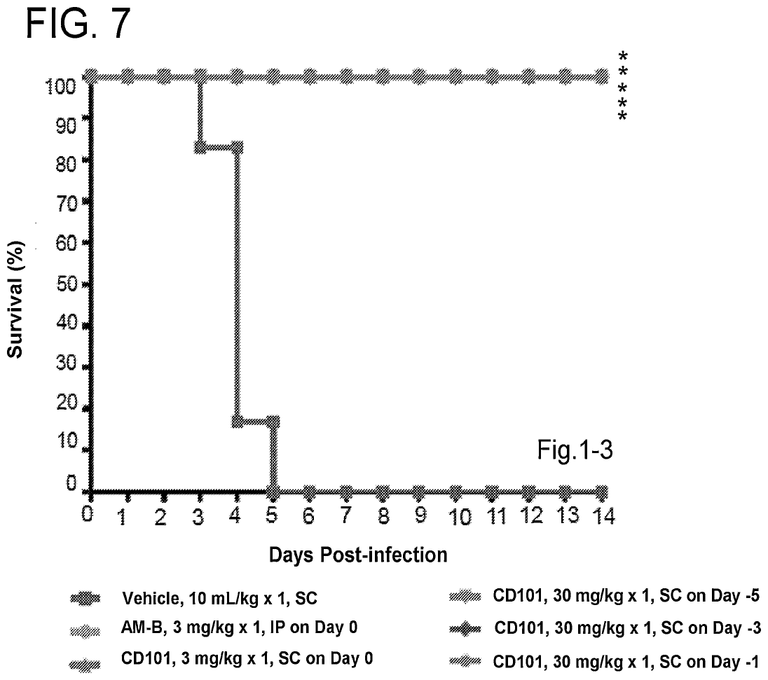

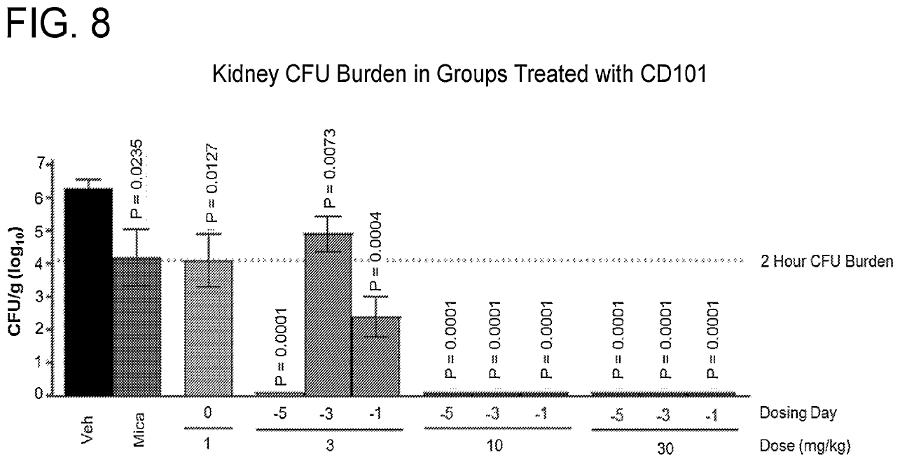

Compared to the vehicle control, all animals showed a significant decrease in CFU count at the 24 hour harvest. Animals receiving 10 mg/kg or 30 mg/kg CD101 at all pre-challenge time points cleared the infection completely by time of harvest (FIG. 8). All but one animal receiving 3 mg/kg CD101 on Day −1 cleared the infection completely. The animals inadvertently given compound at 3 mg/kg on Day −5 and Day −3 completely cleared the infection.

### Example 5. Efficacy CD101 in the Treatment of Candida Auris Infection in a Murine Model of Disseminated Candidiasis

### Methods

Female 6-8 week old CD-1 mice were immunosuppressed with cyclophosphamide (200 mg/kg) 3 days prior to infection and 150 mg/kg 1 day post-infection. On the day of infection, mice were inoculated with 3×10⁷ C. auris blastospores via the lateral tail vein. Mice were randomized into 5 groups (n=5 for colony forming units (CFU) and n=10 for survival): CD101 20 mg/kg administered by intraperitoneal (IP) injection, fluconazole 20 mg/kg administered per os (PO), amphotericin B 0.3 mg/kg IP, and a vehicle control. Treatments were administered 2 hours post-infection (Day 1) and again on Day 4 of the study for a total of 2 doses. Mice were monitored daily and a survival curve was generated. CFU groups were sacrificed on Day 8 of the study. One kidney was removed from each mouse, homogenized, plated on potato dextrose agar (PDA), and incubated at 35° C. for 2 days to determine CFU. The remaining survival mice were monitored until the end of the study (Day 14).

### Results

CD101 showed an average 3 log reduction in kidney CFU compared to fluconazole, amphotericin B, and vehicle treated groups, which was statistically significant (P=0.03, 0.03, and 0.04, respectively). At the end of the study, percent survival of mice in CD101, fluconazole, amphotericin B, vehicle, and untreated groups was 80, 0, 30, 20, and 0%, respectively (FIG. 9).

### Conclusion

Taken together, our findings show that CD101 possesses potent antifungal activity against C. auris infection in a disseminated model of candidiasis. Additionally, treatment with CD101 resulted in a significantly higher overall percent survival.

### Example 6. Evaluate the Ability of CD101 to Prevent and Treat Candida albicans Biofilms and Explore its Temporal Effect by Time Lapse Photography

In this study, we determined the effect of CD101 on prevention and treatment of biofilms formed by Candida albicans in vitro, and evaluated the effect of CD101 (at effective concentration) on formation of biofilm in real time using Time Lapse Microscopy (TLM).

### Materials and Methods

Test Compounds

CD101 powder stocks were reconstituted in water or Yeast Nitrogen Base (YNB) medium, and diluted in YNB to a final working concentration of 0.25 μg/ml and 1 μg/ml. YNB with no CD101 was prepared in parallel and used as controls. Fluconazole was used as a comparator.

Test Media

YNB and Sabouraud dextrose agar (SDA) media

CD101 (powder and reconstituted solution) stored at −80° C. when not in use.

Strains

C. albicans SC-5314 was used for the current study.

Activity of CD101 Against Candida Biofilms

In this study, biofilms were grown in vitro using a biofilm model (Chandra et al., Nature Protocols 3:1909, 2008) and the effect of CD101 on adhesion phase biofilms (representing prevention of biofilms) or mature phase biofilms (representing treatment of biofilms) was determined.

Activity Against Adhesion Phase (Prevention) or Mature Phase (Treatment) Biofilms

Biofilms were formed on silicone elastomer (SE) discs using a catheter-associated-biofilm model (Chandra et al., Nature Protocols 3:1909, 2008; Chandra et al., J. Bacteriol. 183: 5385, 2001; Chandra et al., J. Dental Research 80: 903, 2001). For evaluation of activity against adhesion phase biofilms (prevention), Candida cells were adhered to catheter discs for 90 min. Next, discs were incubated for 24 h with CD101 (0.25 or 1 μg/ml concentrations) to allow biofilm formation. For evaluation of activity against mature phase biofilms (treatment), Candida cells were adhered to catheter discs for 90 min, then transferred to fresh media and incubated for a further 24 h to allow formation of biofilms. Mature biofilms were then exposed to CD101 (0.25 or 1 μg/ml concentrations) for another 24 h. Discs incubated with fluconazole or media alone were used as controls in all experiments.

At the end of drug exposure in both adhesion and mature phase biofilms, biofilms were quantified by measuring their metabolic activity using XTT assay (Chandra et al., Nature Protocols 3:1909, 2008; Chandra et al., J. Bacteriol. 183: 5385, 2001; Chandra et al., J. Dental Research 80: 903, 2001). Following incubation with drugs, discs were transferred to fresh plates containing phosphate buffered saline with XTT and menadione, incubated for 3 h at 37° C. and optical density was read at 492 nm. Separate batches of biofilms were stained with fluorescent dyes (FUN1™, CONA) and observed under Confocal Scanning Laser Microscope (CSLM) to evaluate biofilm architecture and thickness (Chandra et al., Nature Protocols 3:1909, 2008; Chandra et al., J. Bacteriol. 183: 5385, 2001).

Time Lapse Microscopy

The effective CD101 concentration obtained from the above experiments was used to monitor its effect on biofilm formation in real time using TLM, which involves capturing real-time images of a single frame at specific time intervals, allowing temporal monitoring of the interactions occurring between the drug and Candida biofilms. Captured images were combined in a time sequence, resulting in an animation depicting the sequence of events that occurred with the passage of time. Briefly, the discs with C. albicans (adhered for 90 min as above) were placed in a 35-mm-diameter glass-bottom Petri dish (MiTek Corp., Ashland, MA). Next, CD101 (dissolved in the growth medium) was added to the Petri dish, and incubated at 37° C. to allow formation of biofilm. Phase contrast images for this interaction were captured immediately from 0 h and followed up to 16-17 h on a Leica DMI 6000 B inverted microscope connected to a Retiga EXi Aqua camera (Q-imaging Vancouver British Columbia). To determine the structural changes in the maturing biofilm, both acquisition and analysis of a series of horizontal (xy) optical sections of the biofilm was done using Metamorph Imaging software (Molecular Devices, Downington, PA). Disc incubated with media alone was used as control.

Statistical Analyses

Statistical analyses for all data were performed using GraphPad Prism 6 software. Drug treated groups were compared to control untreated groups using unpaired t-tests. P-value of <0.05 was considered significant.

### Results

Activity Against Adhesion Phase Biofilms (Prevention)

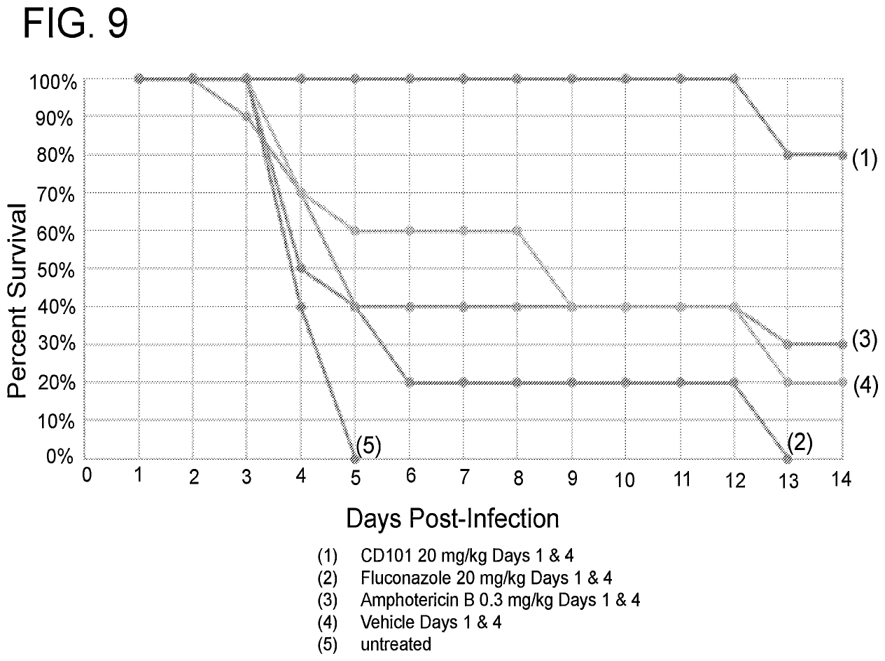

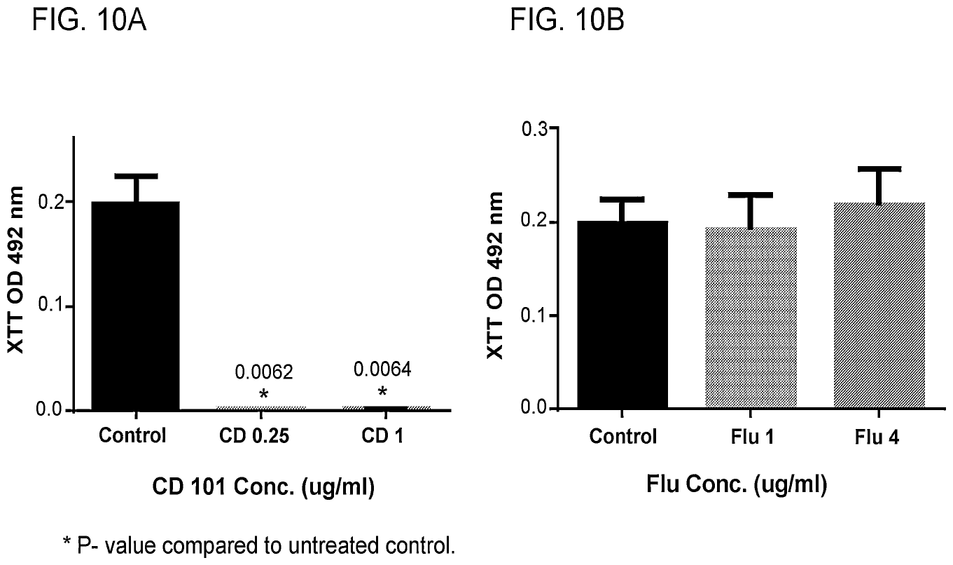

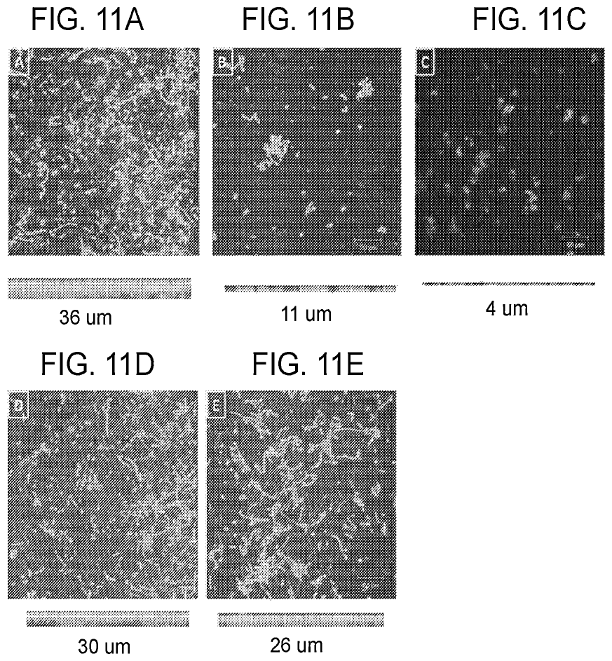

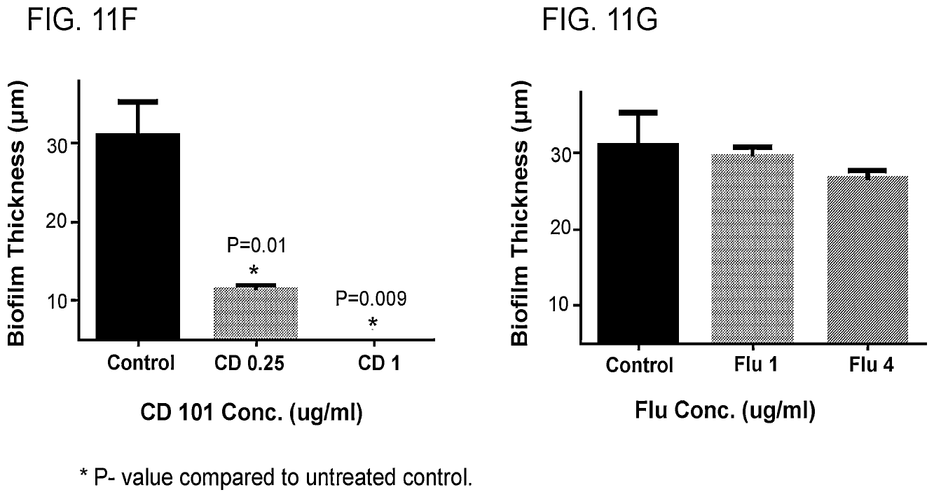

Our metabolic activity and CSLM results showed that CD101 prevented formation of robust biofilms at both concentrations tested (0.25 and 1 μg/ml). Assessment of metabolic activity revealed that C. albicans treated with CD101 formed significantly less biofilms compared to untreated C. albicans (FIG. 10A, P<0.05), In contrast, fluconazole did not inhibit biofilm formation at the two concentrations tested (1 and 4 μg/ml, FIG. 10B, P>0.05). CSLM images showed highly heterogeneous architecture of biofilms with cells/hyphae embedded within extracellular matrix for untreated control (FIG. 11A) while exposure to both concentrations of CD101 showed only remnants of adhered cells, and no biofilm maturation (FIGS. 11B and 11C). In contrast, fluconazole did not inhibit biofilm formation (FIGS. 11D and 11E). Additionally, exposure to CD101 significantly reduced the thickness of biofilms compared to untreated control (36 μm vs. 4 μm, P<0.05, FIG. 11F), while fluconazole had no effect on biofilm thickness (FIG. 11G).

Activity Against Mature Phase Biofilms (Treatment)

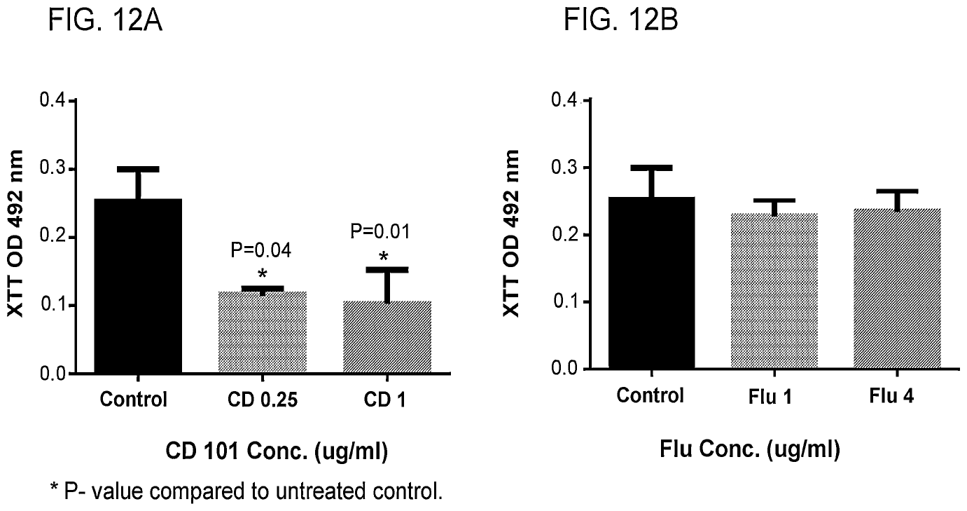

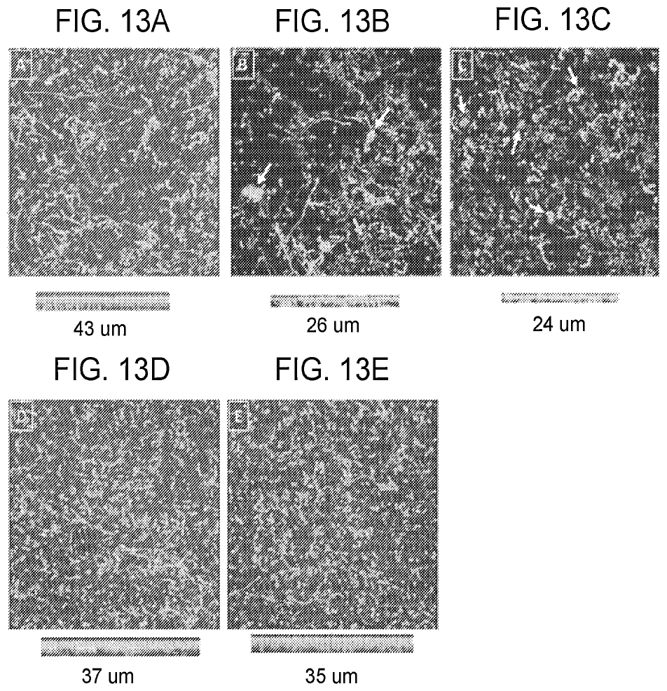

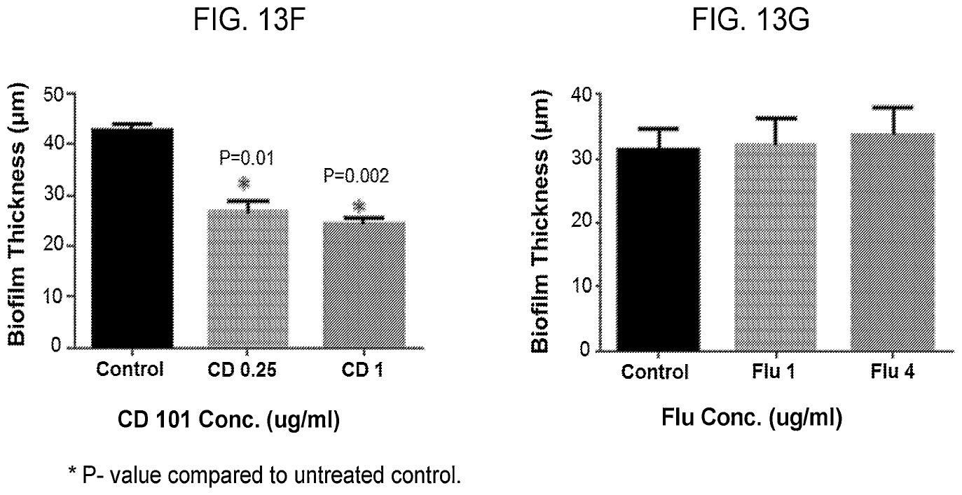

Metabolic activity and CSLM results showed that CD101 was active against mature biofilms at both tested concentrations (0.25 and 1 μg/ml). Mature C. albicans biofilms exposed to CD101 exhibited significantly less metabolic activity compared to those formed by untreated biofilms (FIG. 12A, P<0.05). In contrast, neither concentrations of fluconazole (1 and 4 μg/ml) affected these biofilms (FIG. 12B, P>0.05 compared to untreated controls). CSLM analyses showed highly heterogeneous architecture of biofilms for untreated control (FIG. 13A), while biofilm treated with CD101 were eradicated and showed bulged, deformed/broken cells (FIGS. 13B and 13C). In contrast, fluconazole did not affect Candida biofilms at both concentrations used (FIGS. 13D and 13E). Additionally, CD101 significantly reduced thickness of biofilms compared to untreated control (43 μm vs. 24 μm, P<0.05, FIG. 13F) while fluconazole had no effect (FIG. 13G).

Time Lapse

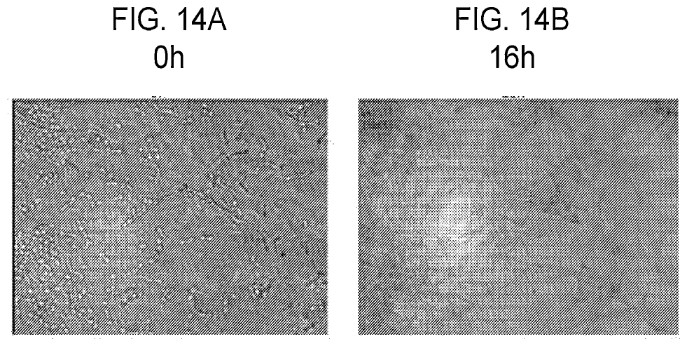

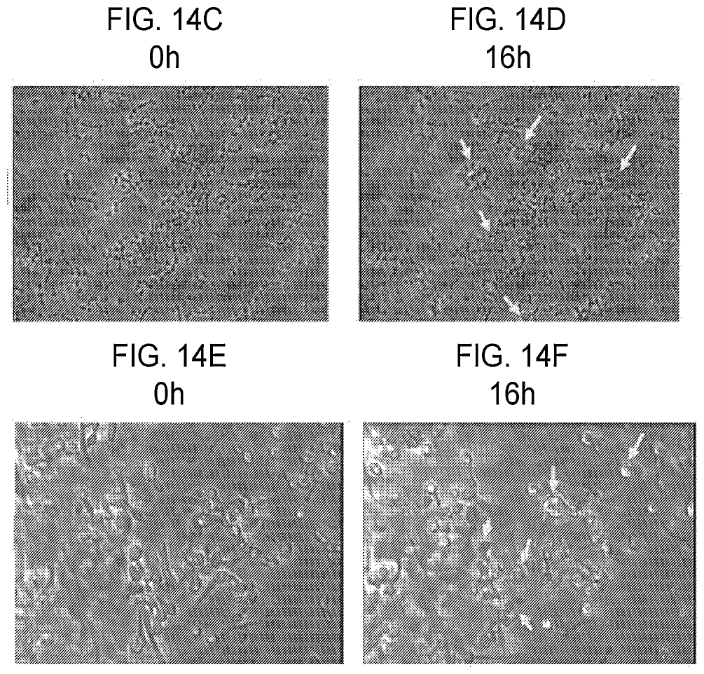

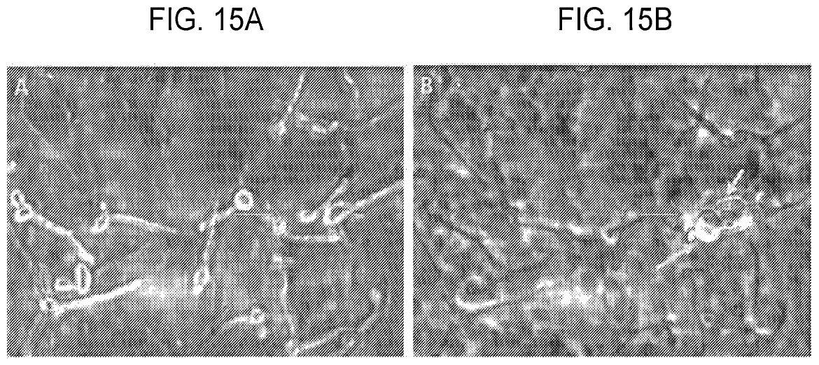

Time lapse movies showed that untreated biofilms formed highly heterogeneous architecture of biofilms with cells/hyphae embedded within extracellular matrix (screen frames in FIGS. 14A and 14B). In contrast, biofilms exposed to 0.25 μg/ml CD101 showed only adhered cells with stunted growth, which failed to grow into mature biofilms (FIGS. 14C-14F). Under high magnification, bulging, deformed, and broken cells were clearly visible (arrows, FIGS. 14D and 14F). The effect of CD101 (0.25 μg/ml) was also studied on 3 h formed biofilms and images were captured immediately after adding the drug and followed up to 16 h. Screen frames in FIG. 15A showed 3 h biofilm hyphal growth which after adding drug remained stunted and failed to grow into mature biofilms (FIG. 15B). Bulged, deformed, broken cells/hypha were clearly visible after 16 h (arrows, FIG. 15B).

### Example 7. Subcutaneous Injection of CD101

Single dose subcutaneous (SC) administration may further extend the utility of CD101 beyond that of other echinocandins, to antifungal treatment and prophylaxis in the outpatient setting. Preclinical studies were conducted to evaluate the feasibility of using SC administration of CD101 for these purposes.

### Methods

The efficacy of CD101 SC was studied in an immunocompetent DBA/2 mouse model of disseminated candidiasis. Mice (5/grp) were challenged with Candida albicans SC5314 (ATCC: MYA-2876, fluconazole-sensitive human clinical isolate shown to be pathogenic in mice) via IV injection (100 μL, 5.0 log CFU/mouse) and treated with CD101 SC (1, 3 or 10 mg/kg). Micafungin via IP administration was tested as a positive control at the same 3 doses. At 24 hours following challenge, kidneys were harvested and processed for CFU enumeration. All comparisons were made between the treatment and time-matched vehicle groups. CD101 SC (5 mg/kg) was also tested in a similar disseminated candidiasis model using ICR mice rendered neutropenic by cyclophosphamide on Day −4 (150 mg/kg) and Day −1 (100 mg/kg) prior to infection by the same C. albicans SC5314 strain (IV injection, 100 μL, 4.5 log CFU/mouse, See Example 1).

Previous toxicology studies by the IV route of administration conducted in cynomolgus monkeys have shown CD101 to be safe and well tolerated at up to at least 30 mg/kg, which generates very high systemic exposures upon initial infusion of CD101 into the bloodstream. Therefore, only local tolerability (and PK) of CD101 by SC administration required evaluation. For this purpose, male and female monkeys were observed for up to 10 days following a single 30 mg/kg SC dose. In the same study, to determine the pharmacokinetics of CD101 following SC administration, whole blood samples were collected and the plasma was harvested at approximately 0.25, 0.5, 1, 2, 4, 8, 24, 36, and 48 hours, and 3, 4, 5, 7, and 10 days postdose. Plasma concentrations were then quantified by liquid chromatography with tandem mass spectrometric detection (LC-MS/MS). Bioavailability from SC dosing was calculated by comparing the calculated area under the concentration-time profile (AUC) from SC against the AUC from IV administration of the same dose.

### Results

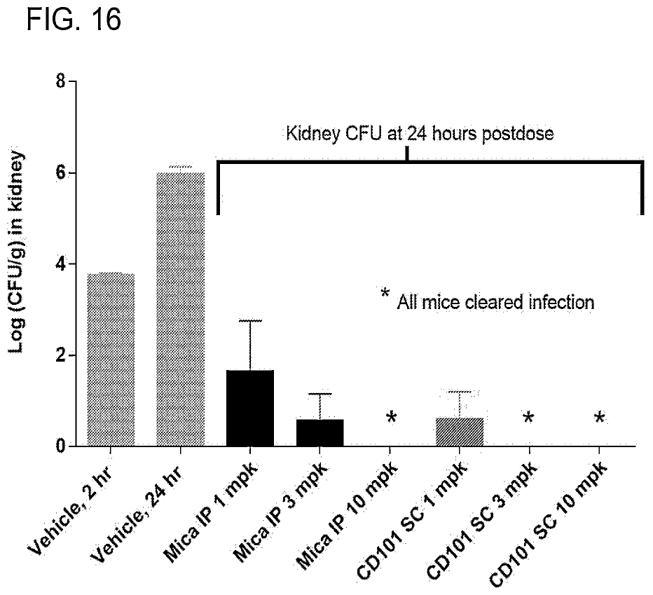

In the DBA/2 mouse efficacy study (FIG. 16), at 2 hr post infection, vehicle-treated mice demonstrated an average kidney CFU of 3.8 log CFU that increased to 6.1 log CFU at 24 hr. Groups treated with CD101 SC (1, 3, and 10 mg/kg) showed significant reduction in kidney CFU when compared with the vehicle control. Animals receiving 3 or 10 mg/kg of CD101 SC showed complete CFU clearance, and 4 of 5 animals in the 1 mg/kg group were completely cleared of CFU burden by 24 hr. Micafungin also showed good dose response in CFU reduction with the same treatment doses but complete clearance was only observed with the 10 mg/kg dose suggesting that it was less efficacious than CD101 at comparable doses.

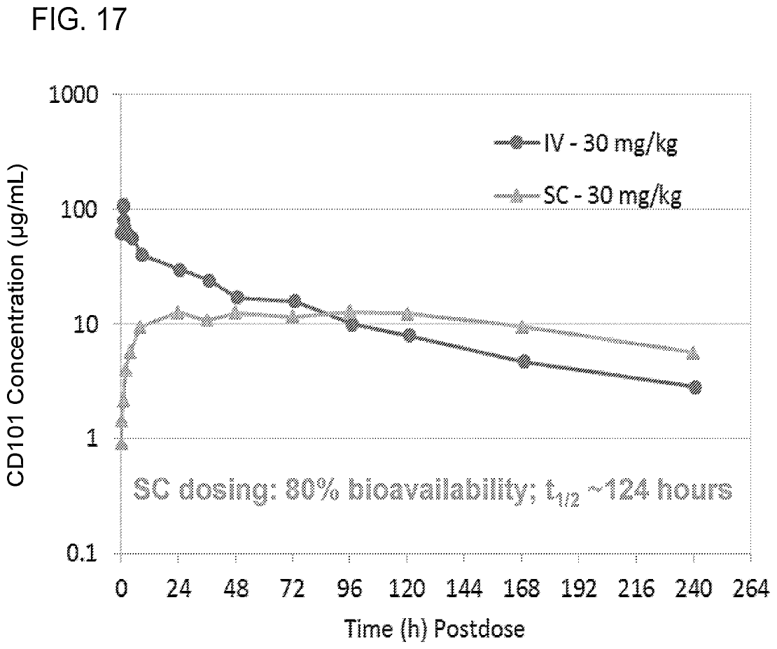

Early pharmacokinetic studies in rats and monkeys had indicated that CD101 subcutaneous administration was well-tolerated, although these initial studies were aimed at characterizing the pharmacokinetics at lower doses (≤5 mg/kg). Therefore, a separate study was designed to evaluate the tolerability of a SC dose of CD101 as a highly concentrated solution (100 mg/mL; 30 mg/kg) in two cynomolgus monkeys. FIG. 17 shows plasma levels of CD101 in two cynomolgus monkeys over 10 days after a single 30 mg/kg dose administered subcutaneously. The drug reaches a maximum concentration within a few hours then remains nearly constant over the course of 10 days. Table 4 further shows various pharmacokinetic characteristics of SC administered CD101 in the monkeys. Table 5 shows the SC formulation of CD101 used in the monkey study.

TABLE 4

| | | Dose | Tmax | Cmax | AUClast | AUCINF_obs | Half-life |
|---|---|---|---|---|---|---|---|
| Animal | Sex | (mg/kg) | (hr) | (ug/mL) | (hr*μg/mL) | (hr*μg/mL) | (hr) |
| 9697 | M | 30 | 96 | 15.9 | 2740 | 3280 | 76.2 |
| 9707 | F | 30 | 48 | 10.9 | 2180 | 3760 | 171 |

TABLE 5

| Component | Function | Concentration |
|---|---|---|
| CD101 acetate | Active ingredient | 100 mg/mL |
| Mannitol | Tonicity | 11.4 mg/mL |
| HCl | pH adjustment | As needed to adjust to pH 5.6 |
| NaOH | pH adjustment | As needed to adjust to pH 5.6 |
| Water for injection | medium | q.s to 1.0 mL |

In the monkey SC tolerability/PK study, no sign of irritation or local (injection site) adverse toxicity was noted following a single high dose of 30 mg/kg CD101. There was also no effect on bodyweight or food consumption upon further follow-up observations for 10 days after administration.

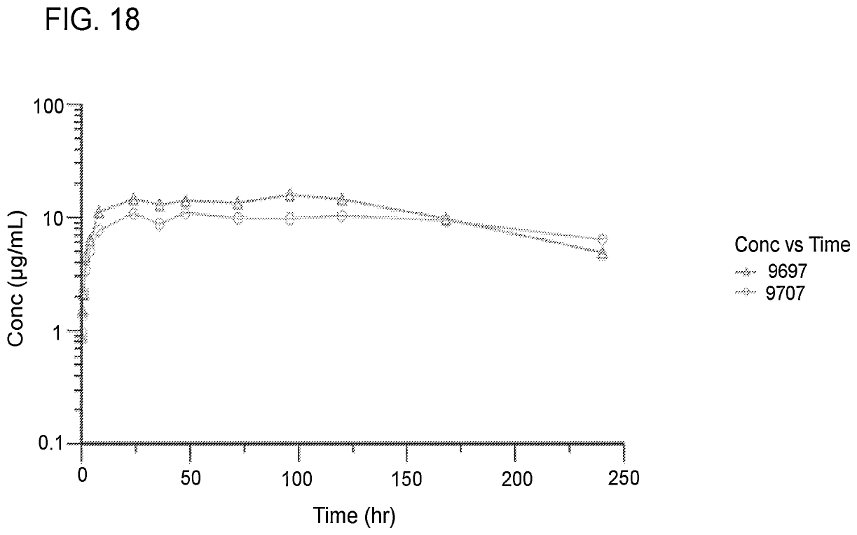

From the same monkey SC tolerability/PK study, the pharmacokinetic profile following SC administration of CD101 at 30 mg/kg showed that total exposure measured over a 10-day period was comparable (80% bioavailability) to that following IV administration at the same dose (FIG. 18), indicating high/equivalent bioavailability from SC administration. The maximum plasma concentration from SC administration was reached after 24 hours and was sustained throughout the first week post-dose. Concentrations started to decrease one week after injection and the terminal half-life estimated was high at approximately 124 hours. Both routes show comparable elimination rates/slopes. Table 6 further shows various pharmacokinetic characteristics of subcutaneously and intravenously administered CD101.

TABLE 6

| | Dose | Tₘₐₓ | Cₘₐₓ | AUCₗₐₛₜ | AUC_{inf} | Half-life |
|---|---|---|---|---|---|---|
| Route | (mg/kg) | (hr) | (μg/mL) | (μg*hr/mL) | (μg*hr/mL) | (hr) |
| Subcutaneous | 30 | 72 | 13.4 | 2460 | 3520 | 124 |
| IV | 30 | 1 | 112 | 3135 | 3340 | 49.7 |

### Example 8. Efficacy of CD101 in the Treatment of Vulvovaginal Candidiasis in a Rat Model

### Methods and Experimental Design

Animal Strain. Wistar rats were supplied by Harlan Laboratories UK and were specific pathogen free. Rats weighed 80-100 g at the time of surgery. Ovariectomies were performed. Rats were allowed 4-7 days recovery before transportation to the facility where the experiment was to be performed. Following arrival, rats were allowed at least 4 days acclimatization before start of the experiment. Rats weighed 100-120 g at the time of ovariectomy and were about 300 g at start of the experiment.

Animal Housing. Rats were housed in sterilized individual ventilated cages that expose the animals at all times to HEPA filtered sterile air. Rats had free access to food and water (sterile) and had sterile aspen chip bedding (changed every 3-4 days). Additionally, during infection, rats had additional access to wet food if required to ensure they remained fully hydrated.

The room temperature was 22° C.+/−1° C., with a relative humidity of 60% and maximum background noise of 56 dB. Mice were exposed to 12 hour light/dark cycles.

Pre-conditioning. Female Wistar rats underwent ovariectomy at least 10 days prior to the study commencing. They were further pre-conditioned by treatment with 5 mg/kg 17-β-estradiol administered subcutaneously (SC) every other day on Days −7, −5, −3 and −1 prior to infection with C. albicans strain 529. Estradiol treatment continued every other day throughout the study to 7 days post-infection.

Yeast Isolate. Candida albicans strain 529L was used in this chronic rat vaginal infection model.

Infection. Yeast strains were inoculated aerobically onto Sabouraud dextrose agar media (SAB) containing 0.05 mg/mL chloramphenicol and incubated at 30° C. for 48-72 h. 18-24 h prior to infection, Yeast Peptone Dextrose (YPD) broths were inoculated with 2-3 isolated colonies from agar plates and incubated overnight (37° C. on an orbital shaker). Broths containing C. albicans strain 529L were washed 3 times with sterile phosphate buffered saline (PBS) before dilution to the correct inoculum for infection. Cell counts were determined using a haemocytometer and confirmed by quantitative culture on Sabouraud dextrose agar.

Rats were infected with 0.1 mL by intravaginal administration under inhaled isoflurane anaesthesia using about 9.8×10⁵ CFU/mL (9.8×10⁴ CFU/Rat) C. albicans strain 529L

Preparation of 17-β-Estradiol, CD101, and Fluconazole

17-β-estradiol in 20% 2-hydroxypropyl-β-cyclodextrin (HPBCD). 90 mg of 17-β-estradiol (Sigma, UK) was weighed and added to 7.2 g HPBCD (Sigma, UK) and water for infection (WFI) added to obtain a final volume of 36 mL of suspension and used immediately to dose animals at 2.5 mg/mL.

Vehicle: 12.81 mg/mL Mannitol in WFI. 384.3 mg of mannitol was weighed and 30 mL of WFI was added. The mixture was briefly vortexed until completely solubilized and was filter sterilized using a 0.2 μm filter. This was stored at 2-8° C. until required and was warmed to room temperature before use.

CD101. To 61.3 mg of CD101 add 12.26 mL of vehicle and mixture briefly vortexed until completely solubilized. This was used neat at 2 mL/kg for the 10 mg/kg dose and was diluted in vehicle 1:2 to prepare the 5 mg/kg dose. These were stored at 2-8° C. until required and were allowed to warm to room temperature before use. Animals were dosed at 2 mL/kg dosing volume by the SC route.

Fluconazole. Clinical oral suspension was used to prepare fluconazole as follows: 1) Oral suspension was prepared as per manufacturer instructions (10 mg/mL Fluconazole); and 2) The 10 mg/mL oral suspension was further diluted 1:5 in WFI to give 2 mg/mL (20 mg/kg) dosing solution. This was maintained at room temperature until required and animals dosed at 10 mg/mL dosing volume orally (by the PO route).

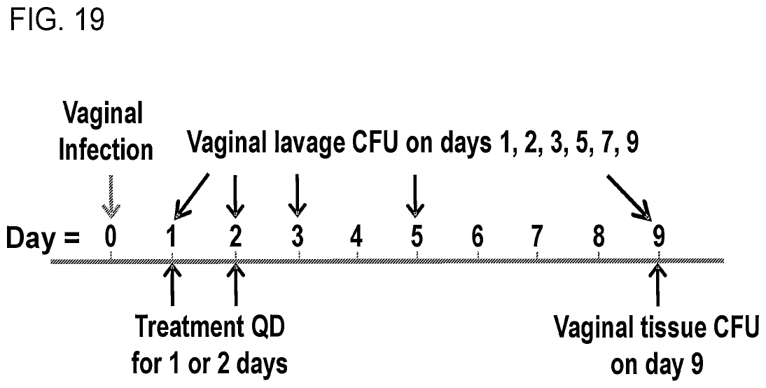

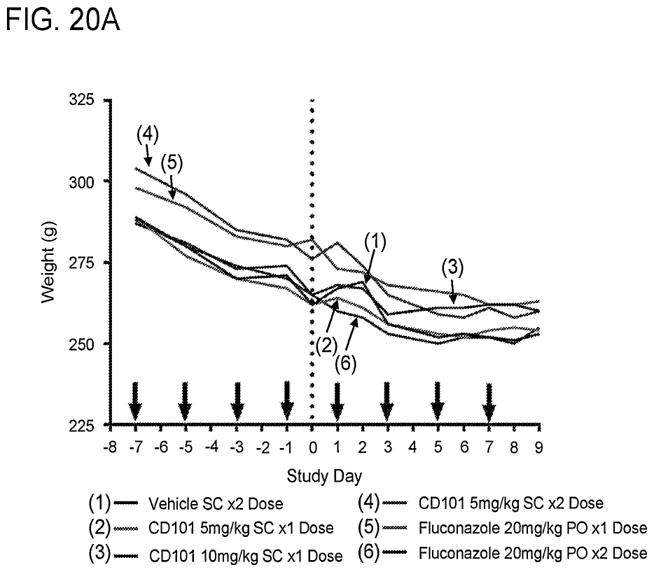

Treatment. CD101, fluconazole, and vehicle treatments started at 24 h post infection by the SC route following the dose volume and frequency shown in Table 7. The fluconazole treatment also started at 24 h post infection but was administered by the PO route at the dose volume and frequency shown in Table 7. The study design is further outlined in FIG. 19.

TABLE 7

| | | | | | | Treatment | | End of | |
|---|---|---|---|---|---|---|---|---|---|
| | | | Dosing | | Number of | commences | Total | Experiment | |
| Group | | Dose | volume | Route/ | treatment | (hours post | Number | (day post | Group |
| No. | Treatment | (mg/kg) | (mL/kg) | Regime | days | infection) | of Doses | infection) | Size |
| 1 | Vehicle | — | 2 | SC OD | 0 | 24 | 2 | 9 | 6 |
| 2 | CD101 | 5 | 2 | SC OD | 1 | 24 | 1 | 9 | 6 |
| 3 | CD101 | 10 | 2 | SC OD | 1 | 24 | 1 | 9 | 6 |
| 4 | CD101 | 5 | 2 | SC OD | 2 | 24 | 2 | 9 | 6 |
| 5 | Fluconazole | 20 | 10 | PO OD | 1 | 24 | 1 | 9 | 6 |
| 6 | Fluconazole | 20 | 10 | PO OD | 2 | 24 | 2 | 9 | 6 |

Endpoints. The rats were monitored at a frequency appropriate for their clinical condition. Rat weights were recorded at least once daily to ensure animals remained within ethical limits.

Rats do not typically succumb to infection in this model but untreated rats may experience some weight loss, dehydration, and piloerection. Reduction in weight and general loss of condition due to estradiol treatment are also typical in this rat model. Colonization with C. albicans was determined by quantitative culture of daily vaginal lavage samples. Rats were euthanized 9 days post infection and C. albicans determined by quantitative culture of vaginal tissue (including uterine horns).

Lavage samples were obtained on Days 1 (pre-treatment), 2, 3, 5, 7, and 9 days post infection by flushing rat vaginas 4 times with 0.1 mL pre-warmed sterile PBS. Following euthanasia, vaginal tissue including uterine horns was removed prior to weighing. Tissues were homogenized in 2 mL sterile PBS using a bead-beater. Vaginal wash and tissue homogenates were diluted appropriately then quantitatively cultured on to Sabouraud dextrose agar containing 0.05 mg/mL chloramphenicol and incubated at 37° C. for up to 72 h before being counted.

Statistical analysis. Data were analyzed using StatsDirect software (version 2.7.8) using the non-parametric Kruskal-Wallis test and if this was statistically significant all pairwise comparisons were analyzed (Conover-Inman).

### Results

In this study, the in vivo efficacy of CD101 dosed SC once at 5 and 10 mg/kg or dosed SC twice at 5 mg/kg was investigated in a rat model of vulvovaginal candidiasis caused by C. albicans strain 529L.

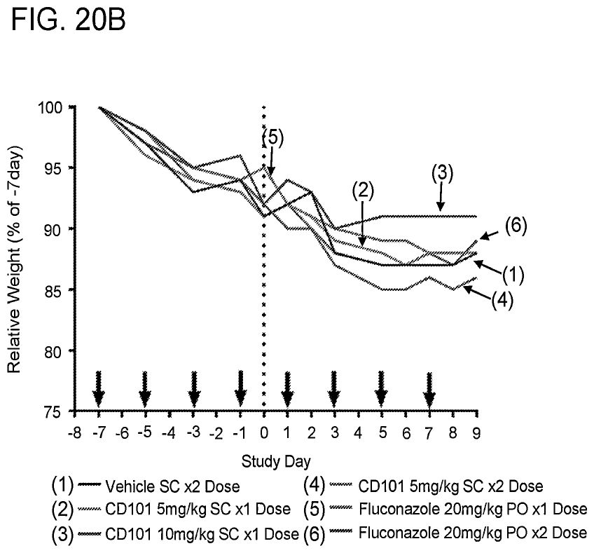

CD101 and Fluconazole Tolerability and Clinical Condition. CD101 and fluconazole at all treatment dose and duration were well tolerated with no adverse events observed. Animal weights following localized vaginal infection with C. albicans and treatment with CD101 or fluconazole are shown in FIGS. 20A and 20B. Animal weights are shown as daily group average weights (FIG. 20A) and the weight relative to that measured on day of infection (Day 0, FIG. 20B). As is typical of this model, ovariectomised rats slowly lost weight following multiple doses of 17-β-estradiol. Weight loss observed was typical of the model and did not seem to be exacerbated by CD101 treatment.

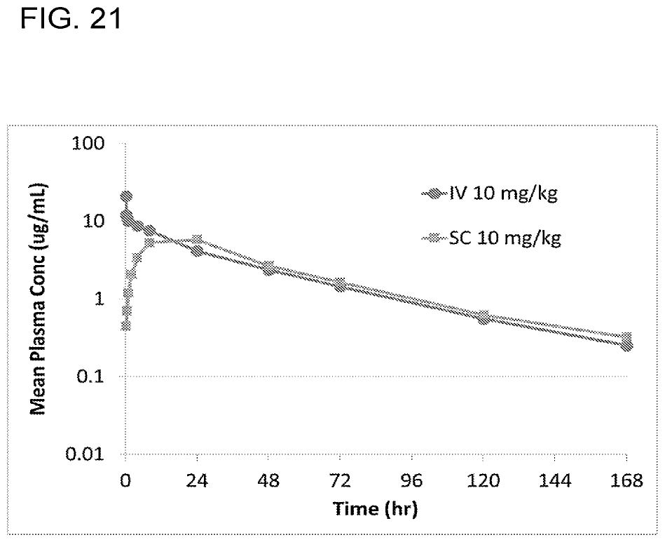

Pharmacokinetic Profile of CD101. The pharmacokinetic (PK) profile of CD101 in female rats (three per group) was characterized. Following subcutaneous (SC) administration, the time to Cmax (i.e. Tmax) was observed between 8 to 24 hours post-dose suggesting slow absorption/distribution from the site of administration (FIG. 21). The half-life, t½, values were similar to those observed from intravenous (IV) dosing and shows a t½ of 48 hrs and SC bioavailability of 97%.

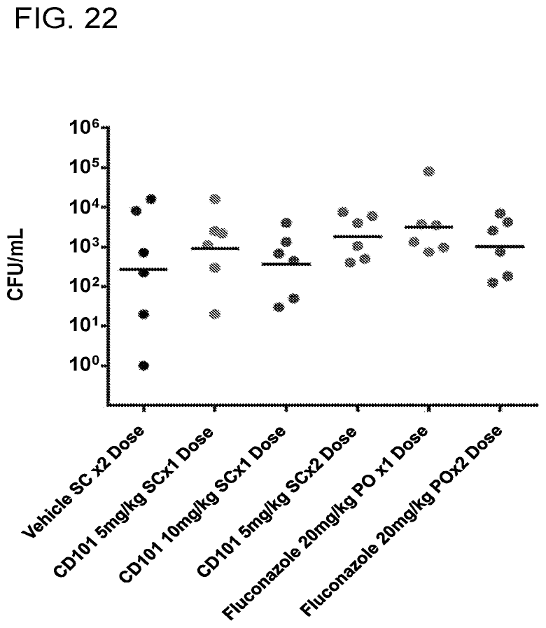

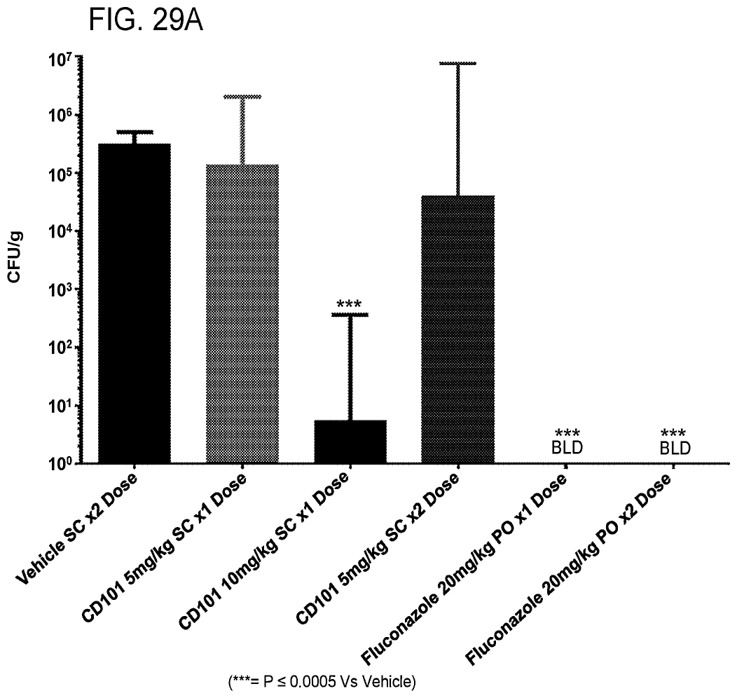

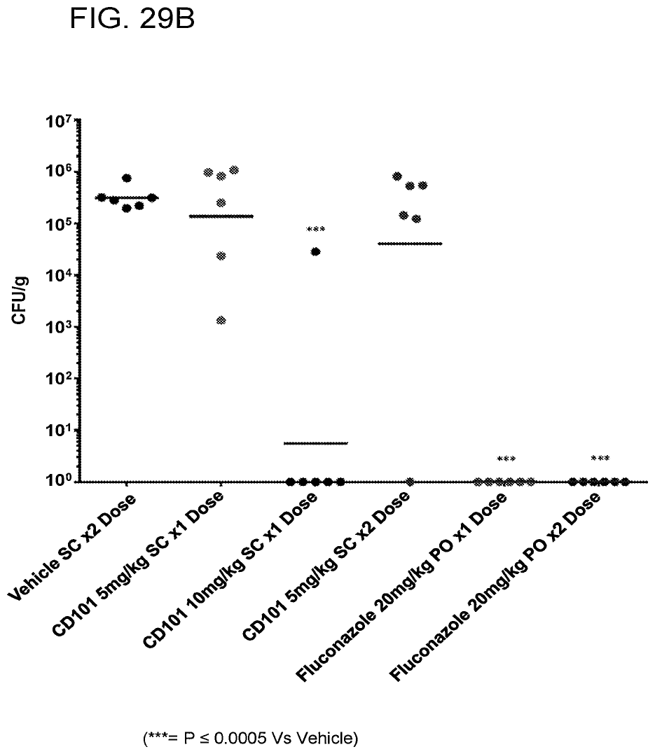

Efficacy Data. A robust model of localized C. albicans strain 529L vaginal infection was successfully established. The geometric mean burden of all infected rats was about 0.9×10³ CFU/mL on Day 1 post infection in pre-treatment vaginal lavage samples (Table 8 and FIG. 22). This level of infection recovered from vehicle only lavage samples increased slightly (about 2.0 Log10 CFU/mL) over the duration of the study and was stable between Day 5 to end of study. A high level (about 5.5 Log10 CFU/g) of C. albicans burden was also obtained from terminal vagina, uterus, and uterine horn tissue taken from vehicle control rats at the end of the study (see Table 14 and FIGS. 29A and 29B) as is typical of this model (the fungi are tightly adherent to the vaginal mucosa due to pseudo-hyphal invasion).

TABLE 8
Geometric mean burden on Day 1 post infection
in pre-treatment vaginal lavage samples

| | | Standard | Log Group | Log reduction |
|---|---|---|---|---|
| | Group Geometric | Deviation | Geometric mean | from vehicle |
| Treatment | mean (CFU/mL) | (CFU/mL) | (CFU/mL) | control (CFU/mL) |
| Vehicle SC - | 2.74 × 10² | 6.60 × 10³ | 2.44 | 0.00 |
| Dosed Twice | | | | |
| CD101 5 mg/kg | 9.11 × 10² | 6.11 × 10³ | 2.96 | −0.52 |
| SC - Dosed Once | | | | |
| CD101 10 mg/kg | 3.68 × 10² | 1.53 × 10³ | 2.57 | −0.13 |
| SC - Dosed Once | | | | |
| CD101 5 mg/kg | 1.83 × 10³ | 3.06 × 10³ | 3.26 | −0.83 |
| SC - Dosed Twice | | | | |
| Fluconazole 20 mg/kg | 3.16 × 10³ | 3.18 × 10⁴ | 3.50 | −1.06 |
| PO - Dosed Once | | | | |
| Fluconazole 20 mg/kg | 1.04 × 10³ | 2.73 × 10³ | 3.02 | −0.58 |
| PO - Dosed Twice | | | | |

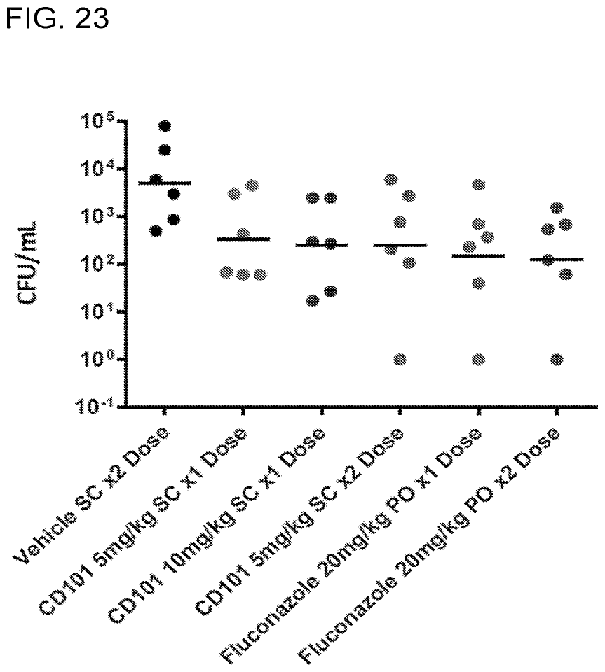

The daily lavage data showed the following results:
Day 1 post treatment (Day 2 post infection, Table 9 and FIG. 23)—All doses of CD101 showed similar reduction in burden (about 1.3 Log₁₀ CFU/mL). Fluconazole showed slightly higher reduction (about 1.6 Log₁₀ CFU/mL). However, neither the CD101 nor fluconazole burden was statistically different from the vehicle burden.

TABLE 9
Geometric mean burden on Day 1 post treatment (Day 2 post infection)

| | | Standard | Log Group | Log reduction |
|---|---|---|---|---|
| | Group Geometric | Deviation | Geometric mean | from vehicle |
| Treatment | mean (CFU/mL) | (CFU/mL) | (CFU/mL) | control (CFU/mL) |
| Vehicle SC - | 5.00 × 10³ | 3.12 × 10⁴ | 3.70 | 0.00 |
| Dosed Twice | | | | |
| CD101 5 mg/kg | 3.34 × 10² | 1.92 × 10³ | 2.52 | 1.17 |
| SC - Dosed Once | | | | |
| CD101 10 mg/kg | 2.48 × 10² | 1.22 × 10³ | 2.39 | 1.30 |
| SC - Dosed Once | | | | |
| CD101 5 mg/kg | 2.56 × 10² | 2.37 × 10³ | 2.41 | 1.29 |
| SC - Dosed Twice | | | | |
| Fluconazole 20 mg/kg | 1.50 × 10² | 1.83 × 10³ | 2.18 | 1.52 |
| PO - Dosed Once | | | | |
| Fluconazole 20 mg/kg | 1.27 × 10² | 5.83 × 10² | 2.10 | 1.59 |
| PO - Dosed Twice | | | | |

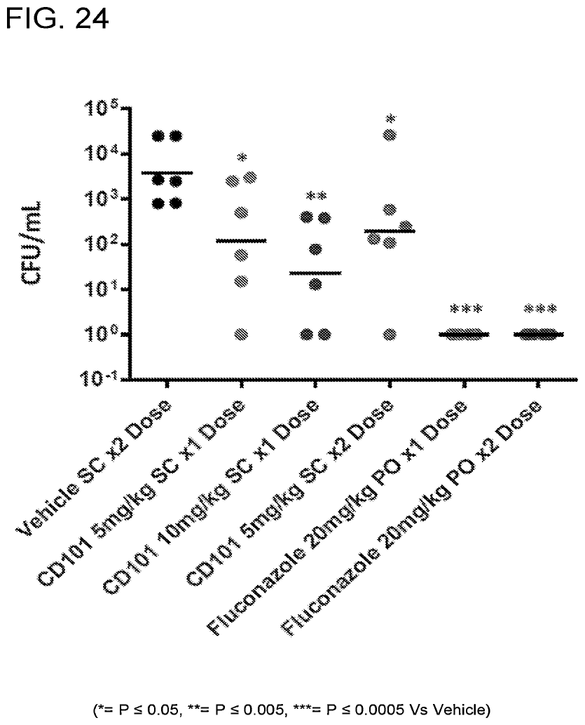

Day 2 posttreatment (Day 3 post infection, Table 10 and FIG. 24)—CD101 dosed once at 10 mg/kg showed a higher reduction in burden compared to CD101 dosed once or twice at 5 mg/kg. The CD101 burden data was more variable compared to vehicle or fluconazole treated animals. Fluconazole showed the greatest reduction in burden with 11/12 rats having burden below the limit of detection. All CD101 and fluconazole treatments were statistically different from the vehicle treatments.

TABLE 10
Geometric mean burden on Day 2 post treatment (Day 3 post infection)

| | | Standard | Log Group | Log reduction |
|---|---|---|---|---|
| | Group Geometric | Deviation | Geometric mean | from vehicle |
| Treatment | mean (CFU/mL) | (CFU/mL) | (CFU/mL) | control (CFU/mL) |
| Vehicle SC - | 3.74 × 10³ | 1.21 × 10⁴ | 3.57 | 0.00 |
| Dosed Twice | | | | |
| CD101 5 mg/kg | 1.21 × 10² | 1.37 × 10³ | 2.08 | 1.49 |
| SC - Dosed Once | | | | |
| CD101 10 mg/kg | 23.2 | 1.92 × 10² | 1.37 | 2.21 |
| SC - Dosed Once | | | | |
| CD101 5 mg/kg | 1.95 × 10² | 1.07 × 10⁴ | 2.29 | 1.28 |
| SC - Dosed Twice | | | | |
| Fluconazole 20 mg/kg | 1.00 | 0.00 | 0.00 | 3.57 |
| PO - Dosed Once | | | | |
| Fluconazole 20 mg/kg | 1.49 | 4.08 | 0.17 | 3.40 |
| PO - Dosed Twice | | | | |

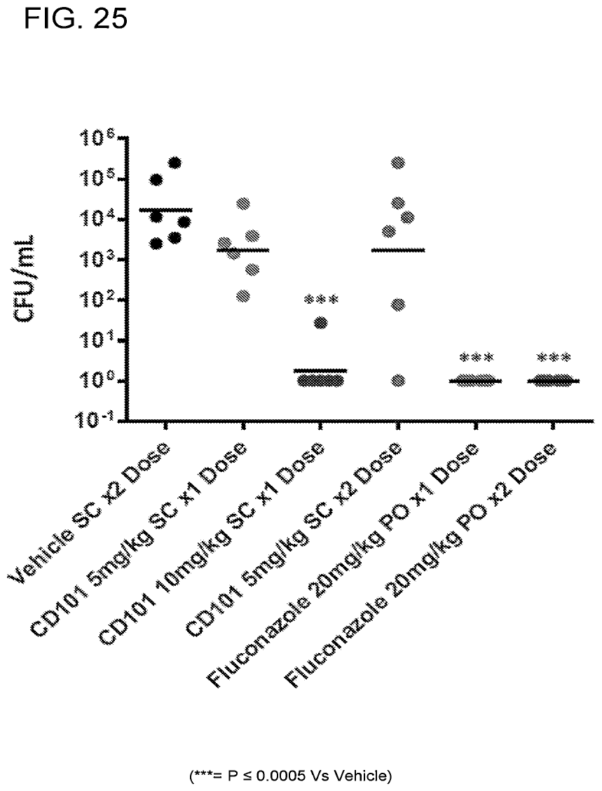

Day 4 post treatment (Day 5 post infection, Table 11 and FIG. 25)—CD101 dosed at 5 mg/kg once or twice reduced the burden to about 1 Log₁₀ CFU/m but this was not statistically different from the vehicle treatments. CD101 dosed once at 10 mg/kg was highly efficacious and reduced the burden to about 4 Logo CFU/mL with 5/6 rats having burden below the limit of detection and almost similar to the fluconazole. All rats treated with the fluconazole once or twice had burdens that were below the limit of detection.

TABLE 11
Geometric mean burden on Day 4 post treatment (Day 5 post infection)

| | | Standard | Log Group | Log reduction |
|---|---|---|---|---|
| | Group Geometric | Deviation | Geometric mean | from vehicle |
| Treatment | mean (CFU/mL) | (CFU/mL) | (CFU/mL) | control (CFU/mL) |
| Vehicle SC - | 1.65 × 10⁴ | 9.88 × 10⁴ | 4.22 | 0.00 |
| Dosed Twice | | | | |
| CD101 5 mg/kg | 1.70 × 10³ | 9.41 × 10³ | 3.23 | 0.99 |
| SC - Dosed Once | | | | |
| CD101 10 mg/kg | 1.73 | 10.6 | 0.24 | 3.98 |
| SC - Dosed Once | | | | |
| CD101 5 mg/kg | 1.73 × 10³ | 9.91 × 10⁴ | 3.24 | 0.98 |
| SC - Dosed Twice | | | | |
| Fluconazole 20 mg/kg | 1.00 | 0.00 | 0.00 | 4.22 |
| PO - Dosed Once | | | | |
| Fluconazole 20 mg/kg | 1.00 | 0.00 | 0.00 | 4.22 |
| PO - Dosed Twice | | | | |

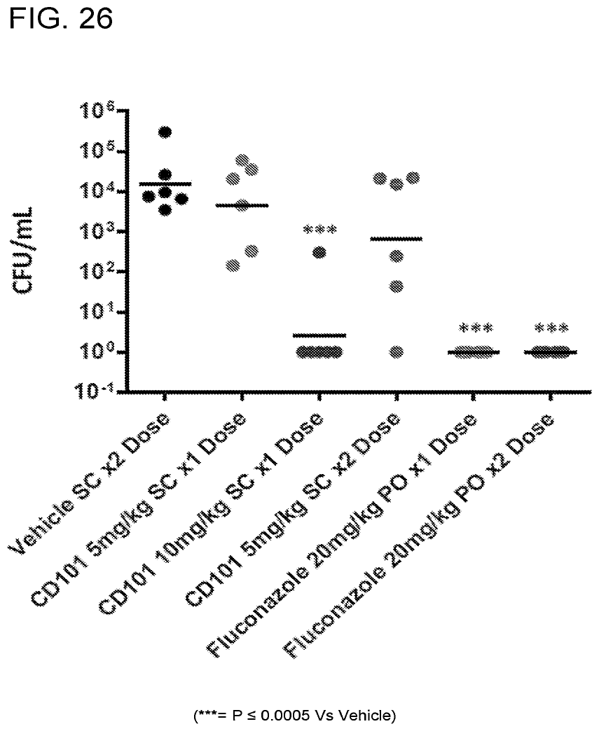

Day 6 post treatment (Day 7 post infection, Table 12 and FIG. 26)—CD101 dosed once at 5 mg/kg showed slight increase in fungal burden compared to Day 5 post infection. CD101 dosed twice at 5 mg/kg reduced the burden more than Day 5 post infection but was not statistically significant. CD101 dosed at 10 mg/kg was similar to Day 5 post infection but the overall burden reduction was not as high as Day 5. The single rat with detectable burden on Day 5 had a slightly increased fungal burden.

TABLE 12
Geometric mean burden on Day 6 post treatment (Day 7 post infection)

| | | Standard | Log Group | Log reduction |
|---|---|---|---|---|
| | Group Geometric | Deviation | Geometric mean | from vehicle |
| Treatment | mean (CFU/mL) | (CFU/mL) | (CFU/mL) | control (CFU/mL) |
| Vehicle SC - | 1.53 × 10⁴ | 1.18 × 10⁵ | 4.18 | 0.00 |
| Dosed Twice | | | | |
| CD101 5 mg/kg | 4.56 × 10³ | 2.39 × 10⁴ | 3.66 | 0.52 |
| SC - Dosed Once | | | | |
| CD101 10 mg/kg | 2.59 | 1.22 × 10² | 0.41 | 3.77 |
| SC - Dosed Once | | | | |
| CD101 5 mg/kg | 6.47 × 10² | 1.08 × 10⁴ | 2.81 | 1.37 |
| SC - Dosed Twice | | | | |
| Fluconazole 20 mg/kg | 1.00 | 0.00 | 0.00 | 4.18 |
| PO - Dosed Once | | | | |
| Fluconazole 20 mg/kg | 1.00 | 0.00 | 0.00 | 4.18 |
| PO - Dosed Twice | | | | |

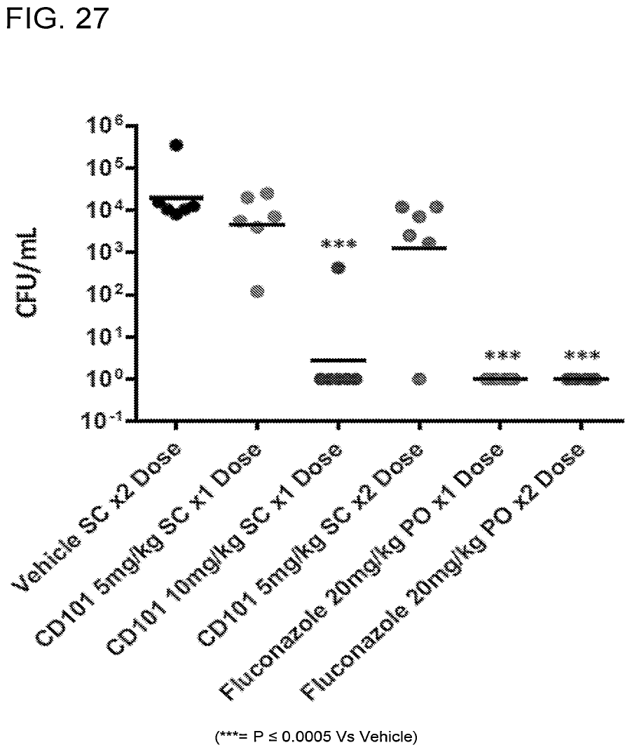

Day 8 post treatment (Day 9 post infection, Table 13 and FIG. 27)—Data were similar to Day 6 post treatment.

TABLE 13
Geometric mean burden on Day 8 post treatment (Day 9 post infection)

| | | Standard | Log Group | Log reduction from |
|---|---|---|---|---|
| | Group Geometric | Deviation | Geometric mean | vehicle control |
| Treatment | mean (CFU/mL) | (CFU/mL) | (CFU/mL) | (CFU/mL) |
| Vehicle SC - | 1.98 × 10⁴ | 1.38 × 10⁵ | 4.30 | 0.00 |
| Dosed Twice | | | | |
| CD101 5 mg/kg | 4.58 × 10³ | 9.87 × 10³ | 3.66 | 0.64 |
| SC - Dosed Once | | | | |
| CD101 10 mg/kg | 2.75 | 1.76 × 10² | 0.44 | 3.86 |
| SC - Dosed Once | | | | |
| CD101 5 mg/kg | 1.28 × 10³ | 5.28 × 10³ | 3.11 | 1.19 |
| SC - Dosed Twice | | | | |
| Fluconazole 20 mg/kg | 1.00 | 0.00 | 0.00 | 4.30 |
| PO - Dosed Once | | | | |
| Fluconazole 20 mg/kg | 1.00 | 0.00 | 0.00 | 4.30 |
| PO - Dosed Twice | | | | |

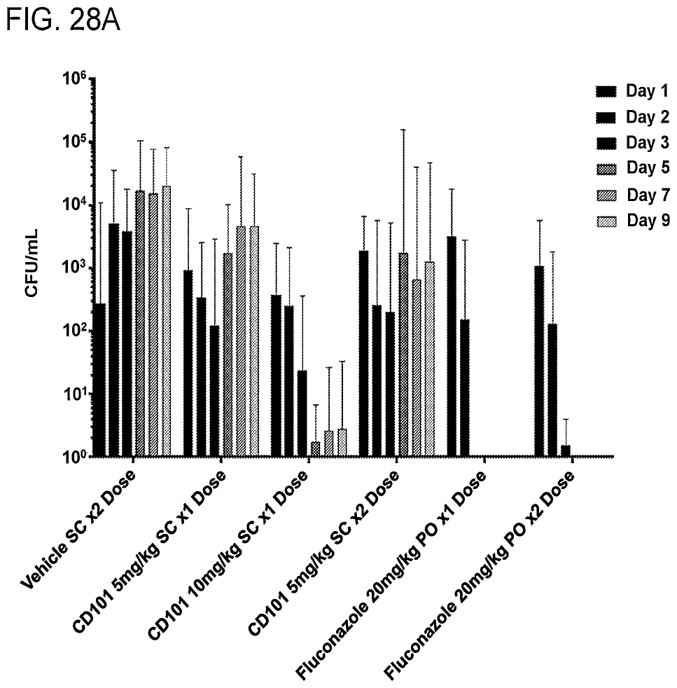

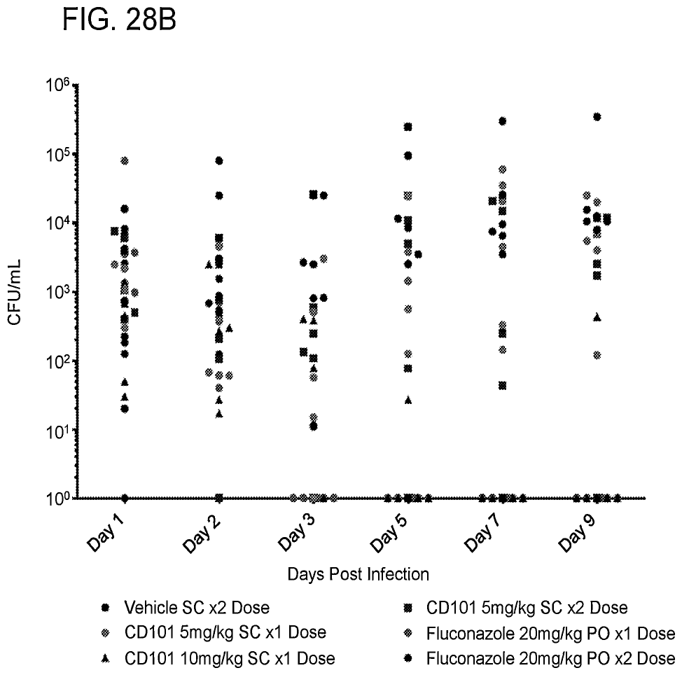

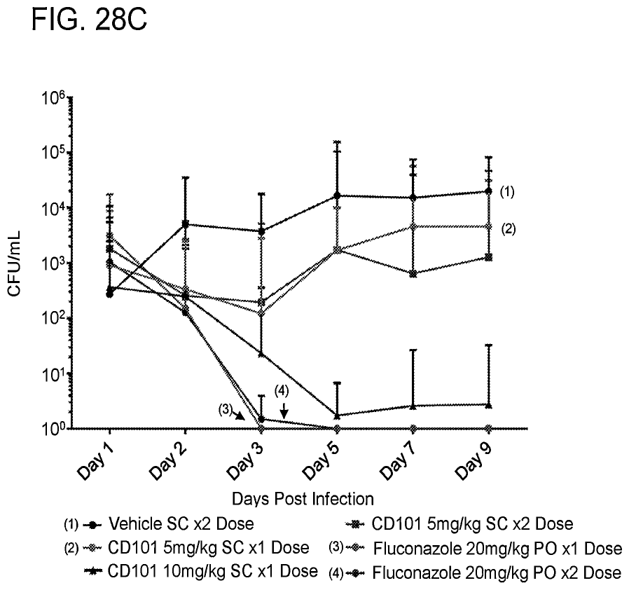

The lavage burden data are summarized in FIGS. 28A-28C. A robust VVC model was established (FIG. 28C); vehicle-treated rats maintained a high fungal burden throughout the study, rising to 2×10⁴ CFU/mL by Day 9 post infection. CD101 administered once at 10 mg/kg was the most effective dose and similar to fluconazole dosed at 20 mg/kg showing comparable CFU by Day 5 post infection and thereafter the burden increased slightly. The increase was caused by a single rat that had a small fungal burden on Day 5 but which increased on Day 7 and 9 post infection. As expected, Day 9 tissue CFU were higher than lavage CFU for all treatments, but the overall pattern was similar to lavage CFU. All but one rat treated with CD101 once at 10 mg/kg had undetectable CFU.

The terminal vaginal tissue burdens (vagina, uterus, and uterine horns) are shown in Table 14 and FIGS. 29A and 29B. The data is in line with that observed in the vaginal lavage washes. The data showed that CD101 dosed once at 5 mg/kg resulted in the smallest reduction in burden (about 0.4 Log₁₀ CFU/g) followed by CD101 dosed twice at 5 mg/kg (about 0.9 Log₁₀ CFU/g), neither were statistically lower than vehicle treatments. 5/6 rats treated with CD101 dosed once at 10 mg/kg had burdens below the levels of detection. A single rat had low level of burden. All rats treated with fluconazole once or twice had burdens below the limit of detection.

TABLE 14
Terminal vaginal tissue burden (vagina, uterus, and uterine horns) (Day +9 post infection)

| | | Standard | Log Group | Log reduction from |
|---|---|---|---|---|
| | Group Geometric | Deviation | Geometric mean | vehicle control |
| Treatment | mean (CFU/g) | (CFU/g) | (CFU/g) | (CFU/g) |
| Vehicle SC - | 3.11 × 10⁵ | 2.05 × 10⁵ | 5.49 | 0.00 |
| Dosed Twice | | | | |
| CD101 5 mg/kg | 1.37 × 10⁵ | 4.88 × 10⁵ | 5.14 | 0.36 |
| SC - Dosed Once | | | | |
| CD101 10 mg/kg | 5.52 | 1.16 × 10⁴ | 0.74 | 4.75 |
| SC - Dosed Once | | | | |
| CD101 5 mg/kg | 4.00 × 10⁴ | 3.16 × 10⁵ | 4.60 | 0.89 |
| SC - Dosed Twice | | | | |
| Fluconazole 20 mg/kg | 1.00 | 0.00 | 0.00 | 5.49 |
| PO - Dosed Once | | | | |
| Fluconazole 20 mg/kg | 1.00 | 0.00 | 0.00 | 5.49 |
| PO - Dosed Twice | | | | |

### Summary

A robust model of localized rat chronic model of vulvovaginal candidiasis following infection with C. albicans strain 529L was established. The infectious burden peaked to >4 Log₁₀ CFU/mL in lavage samples from the vehicle treatment group. A high level of C. albicans burden was also recovered from vagina, uterus, and uterine horn tissue taken from vehicle control rats.

Treatment with CD101 administered by the SC route showed the following in lavage wash burdens:
A single dose of 5 mg/kg administered 24 h post infection reduced fungal burden with a peak effect at Day 3 post infection (48 h post treatment) but which was not maintained to the end of the study. Burden reduction was statistically significant vs. vehicle controls only at Day 3 post infection.
A single dose of 5 mg/kg given twice; once at 24 h and another at 48 h post infection resulted in a superior burden reduction compared to the single dose which was maintained for the duration of the study. But like the single dose, statistical significance was only observed at Day 3 post infection.
A single dose at 10 mg/kg (equivalent to 200 mg in human) given at 24 h post infection resulted in substantial reduction in fungal burden at 3 day post infection (48 h post treatment) with a peak effect at 5 day post infection. Thereafter burden appeared to increase again but this was due to a single rat that retained burden whereas all others had actually burdens below the level of detection. These results suggest excellent CD101 distribution/penetration into vaginal mucosa via a single SC dose. Treatment with CD101 administered by the SC route showed the following vaginal tissue burden:
A single dose at 5 mg/kg 24 h post infection resulted in a slight reduction in burden.
A single dose at 5 mg/kg given twice; once at 24 h and another at 48 h post infection (total of 2 doses) resulted in a larger decrease in burden compared to the single dose.
A single dose at 10 mg/kg (equivalent to 200 mg in human) given at 24 h post infection resulted in substantial reduction in burden with clearance in fungal burden to below the level of detection in 5 of the 6 rats. Similar to the lavage data, a single rat retained fungal burden. These results suggest excellent CD101 distribution/penetration into vaginal mucosa via a single SC dose.

All rats treated with fluconazole at 20 mg/kg PO dosed once at 24 h post infection or single dose twice (24 h and 48 h post infection) showed a faster reduction in burden from lavage washes compared to CD101. At the end of the study, all rats had cleared the infection to below the level of detection in both the lavage wash and vaginal tissue.

### Example 9. Efficacy of CD101 in a Murine Model of Pulmonary Aspergillosis

This study assessed the antifungal efficacy of CD101 by intraperitoneal administration in a murine model of pulmonary aspergillosis caused by Aspergillus fumigatus (strain AF293) compared to micafungin. The primary objective of the study was to compare survival between the treatment groups.

### Methods

Animal Strain and Housing. Mice used in these studies were supplied by Charles River (Margate UK) and were specific pathogen free. The strain of mice used was ICR (also known as CD1 Mice) which is a well characterized outbred murine strain. Male mice were 11-15 g on receipt at our facility and allowed to acclimatize for at least seven days.

Immunosuppression. Mice were immunosuppressed on Day −4 with 150 mg/kg cyclophosphamide IP, and on Day −1 with 150 mg/kg cyclophosphamide IP and 175 mg/kg cortisone acetate SC. To prevent bacterial infection due to the immunosuppression mice were given 50 mg/kg/day ceftazidime.

Preparation of Organism and Infection. A. fumigatus strain AF293 inoculum was prepared from spore cultures grown on Sabouraud Dextrose agar (SAB) containing 50 μg/mL chloramphenicol (SABC) in vented tissue culture flasks. Following incubation for 7-10 days at 30° C., spore cultures were washed in sterile phosphate buffered saline (PBS) containing 0.05% Tween 80. Spore count was determined using a haemocytometer and spores were diluted in PBS to ˜6.9×10⁶ CFU/mL. Inoculum concentration was confirmed by quantitative culture onto SABC agar. Neutropenic mice lungs were infected with 0.04 mL (0.02 mL/nares) of ˜6.9×10⁶ CFU/mL (˜2.8×10⁵ cfu/mouse) of A. fumigatus strain AF293 by intranasal (IN) instillation under temporary 2.5% isoflurane induced anesthesia.

Preparation of Test Articles. Micafungin was provided as a 50 mg vial (Lot 02323002, expiry 08/2017) and was prepared as per manufacturer instructions by adding 5 mL saline for injection (SFI) directly into the vial to make a 10 mg/mL stock solution. This solution was then diluted further in SFI to a working concentration of 0.2 mg/mL. The compound was administered IP at 10 mL/kg to achieve a 2 mg/kg dose. It was prepared fresh once and stored at 4° C. between doses.

Vehicle and CD101 diluent was 10⁰% DMSO/1% Tween 20 in SFI: 1 mL of Tween 20 was added to 10 mL DMSO and the gently mixed and SFI added to a final volume of 100 mL. This was filter sterilised and maintained at room temperature before use for dosing or formulating CD101. The vehicle was administered IP at 10 mL/kg.

Test article CD101 stock was prepared at 2 mg/mL in 10% DMSO/1% Tween 20 diluent. A clear non particulate solution was obtained following gentle mixing. The stock was kept at 4° C. until required. Study doses of 5 mg/kg (0.5 mg/mL) and 10 mg/kg (1 mg/mL) were prepared from the 2 mg/mL stock as required by diluting in 10% DMSO/1% Tween 20 diluent. The 2 mg/mL stock was used undiluted for the 20 mg/kg study dose. All doses were administered IP at 10 mL/kg.

Treatment. For this study, treatments were initiated on day five pre-infection according to treatment groups outlined in Table 15. A total of 78 mice (six mice per treatment group) were used in the study.

TABLE 15
Murine model of pulmonary aspergillosis treatment groups

| | Test | | | Dosing | Conc. mg/mL | | | Total | Mice | End of |
|---|---|---|---|---|---|---|---|---|---|---|
| Group | Article | Route | Schedule | Day | ml/kg | mg/kg | Dosage | Dose | (ICR) | study |
| 1 | Vehicle | IP | Single | −5 | — | — | — | — | 6 | 10 |
| 2 | Micafungin | IP | Single | 0* | 0.2 | 10 | 2 | 2 | 6 | 10 |
| 3 | Micafungin | IP | Single | −1 | 0.2 | 10 | 2 | 2 | 6 | 10 |
| 4 | CD101 | IP | Single | 0* | 0.5 | 10 | 5 | 5 | 6 | 10 |
| 5 | CD101 | IP | Single | −1 | 0.5 | 10 | 5 | 5 | 6 | 10 |
| 6 | CD101 | IP | Single | −3 | 0.5 | 10 | 5 | 5 | 6 | 10 |
| 7 | CD101 | IP | Single | −5 | 0.5 | 10 | 5 | 5 | 6 | 10 |
| 8 | CD101 | IP | Single | −1 | 1 | 10 | 10 | 10 | 6 | 10 |
| 9 | CD101 | IP | Single | −3 | 1 | 10 | 10 | 10 | 6 | 10 |
| 10 | CD101 | IP | Single | −5 | 1 | 10 | 10 | 10 | 6 | 10 |
| 11 | CD101 | IP | Single | −1 | 2 | 10 | 20 | 20 | 6 | 10 |
| 12 | CD101 | IP | Single | −3 | 2 | 10 | 20 | 20 | 6 | 10 |
| 13 | CD101 | IP | Single | −5 | 2 | 10 | 20 | 20 | 6 | 10 |

*1 h post infection

General Health monitoring. The mice were monitored at a frequency appropriate for their clinical condition. Mouse weights were recorded at least once daily both to ensure animals remained within ethical limits and to monitor efficacy of treatment.

Endpoints. The primary endpoint for this study was survival within agreed ethical limits (>20% weight loss, severe hypothermia <34° C., inability to reach food or drink, severe hunching). Mice were monitored by daily weight measurements with observations as frequently as clinical condition required. Mice presenting with severe clinical deterioration were humanely euthanized using an overdose of pentobarbitone administered by IP injection following clinical assessment and the time of death was recorded. Animal carcasses were stored at −20° C. for assessment of burden. Ten days post infection all surviving animals were weighed and had their clinical condition assessed prior to being euthanized. Final survival numbers were recorded and analyzed as described below and carcasses frozen at −20° C. prior to further processing.

A secondary endpoint for the study was terminal lung tissue burden. Immediately following confirmation of death, carcasses were frozen at −20° C. prior to tissue dissection and processing. The frozen carcasses were thawed at room temperature and the lungs removed and placed into pre-weighed bead-beating tubes containing 2 mL of PBS and subjected to mechanical disruption. Organ homogenates were diluted further in PBS and quantitatively cultured for A. fumigatus onto SABC and incubated at 30° C. for 24-48 hours. In addition, a 300 μL aliquot of the undiluted lung tissue homogenate was stored at −80° C. for possible optional assessment of burden by qPCR.

Statistical analysis. Data were analyzed using StatsDirect software (version 2.7.8). Survival data were analyzed using the Kaplan Meier and Log-Rank and Wilcoxon tests (using the Peto-Prentice weighting method).

### Results

The aim of this study was to determine the in vivo efficacy of CD101 in a murine model of pulmonary aspergillosis. The design of this study is summarized in Table 15. All treatments were well tolerated with no adverse signs observed.

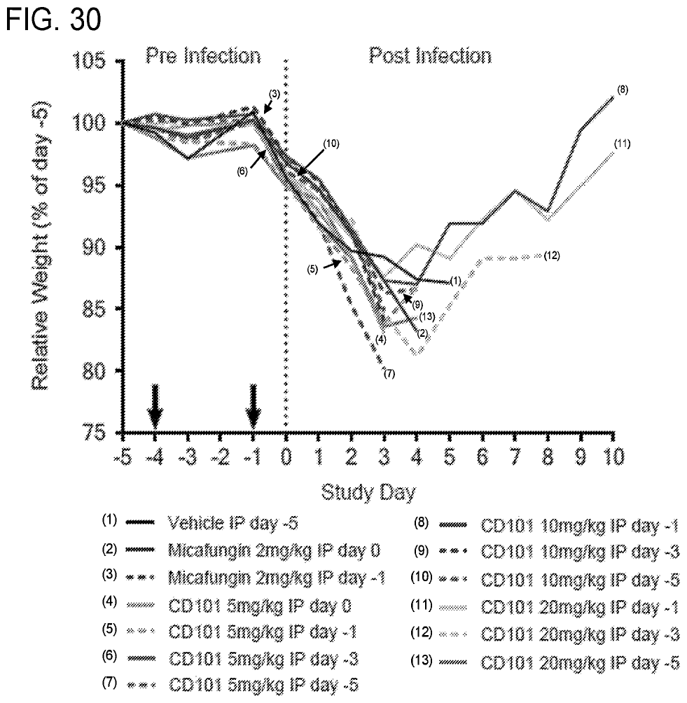

Body weights. Animal weights following infection with A. fumigatus strain AF293 are shown in FIG. 30. Animal weights are shown relative to the weight on Day −5 pre-infection (first treatment time). Weights remained stable up to Day −1 pre-infection. Mice from all treatment groups lost weight following the immunosuppression on Day −1. The weight loss continued after the infection in almost all treatments groups except mice treated with CD101 at 10 mg/kg on Day −1 and CD101 at 20 mg/kg on Day −3 and Day −1 from three days post infection.

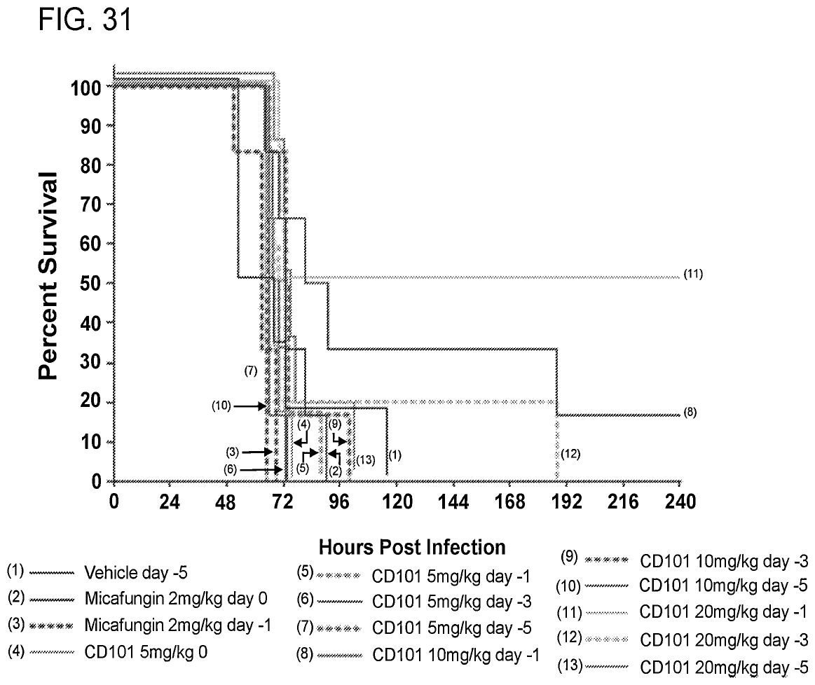

Survival. The median and mean survival for the various treatments are shown in Table 16 and the survival plots for all treatment groups are shown in FIG. 31. Statistical outcomes from the Log-Rank and Wilcoxon test are shown in Table 17.

TABLE 16
Mean and median survival per treatment group

| | Median | Mean |
|---|---|---|
| | Survival | Survival |
| Treatment | (Hours) | (Hours) |
| Vehicle IP Day −5 | 52.5 | 69.0 |
| Micafungin 2 mg/kg IP Day 0 | 73.3 | 75.3 |
| Micafungin 2 mg/kg IP Day −1 | 64.7 | 64.7 |
| CD101 5 mg/kg IP Day 0 | 64.6 | 67.8 |
| CD101 5 mg/kg IP Day −1 | 67.6 | 72.1 |
| CD101 5 mg/kg IP Day −3 | 67.7 | 70.0 |
| CD101 5 mg/kg IP Day −5 | 65.3 | 65.3 |
| CD101 10 mg/kg IP Day −1 | 81.0 | 121.8 |
| CD101 10 mg/kg IP Day −3 | 73.4 | 76.9 |
| CD101 10 mg/kg IP Day −5 | 65.7 | 67.2 |
| CD101 20 mg/kg IP Day −1 | 72.3 | 155.6 |
| CD101 20 mg/kg IP Day −3 | 69.8 | 91.9 |
| CD101 20 mg/kg IP Day −5 | 69.8 | 75.5 |

A robust survival model of pulmonary aspergillosis infection with A. fumigatus strain AF293 was established. Vehicle treated mice started to succumb to the infection ˜48 h post infection and all had succumbed to the infection by day 5 post infection resulting in a mean survival time of 69 h post infection. The study was terminated 10 days post infection as most mice had succumbed to the infection.

Survival in animal groups treated with CD101 was compared against groups treated with the comparator micafungin at the same time pre- or post-infection. Groups treated with 10 mg/kg and 20 mg/kg CD101 one day before infection had statistically greater survival compared to groups treated with micafungin one day before infection (Table 17).

TABLE 17
Log-rank and Wilcoxon test output for different comparisons

| Comparison | Log-Rank | Peto-Prentice |
|---|---|---|
| Vehicle vs. Micafungin 2 mpk Day 0 | NS | NS |
| Vehicle vs. Micafungin 2 mpk Day −1 | NS | NS |
| Vehicle vs. CD101 5 mpk Day 0 | NS | NS |
| Vehicle vs. CD101 5 mpk Day −1 | NS | NS |
| Vehicle vs. CD101 5 mpk Day −3 | NS | NS |
| Vehicle vs. CD101 5 mpk Day −5 | NS | NS |
| Vehicle vs. CD101 10 mpk Day −1 | NS | NS |
| Vehicle vs. CD101 10 mpk Day −3 | NS | NS |
| Vehicle vs. CD101 10 mpk Day −5 | NS | NS |
| Vehicle vs. CD101 20 mpk Day −1 | NS (0.0533) | 0.0465 |
| Vehicle vs. CD101 20 mpk Day −3 | NS | NS |
| Vehicle vs. CD101 20 mpk Day −5 | NS | NS |
| Micafungin 2 mpk Day 0 vs. CD101 5 mpk Day 0 | NS | NS |
| Micafungin 2 mpk Day −1 vs. CD101 5 mpk Day −1 | NS | 0.0467 |
| Micafungin 2 mpk Day −1 vs. CD101 10 mpk Day −1 | 0.0201 | 0.0191 |
| Micafungin 2 mpk Day −1 vs. CD101 20 mpk Day −1 | 0.0047 | 0.0067 |
| CD101 5 mpk Day −1 vs. CD101 10 mpk Day −1 | NS | NS |
| CD101 5 mpk Day −1 vs. CD101 20 mpk Day −1 | NS | NS |
| CD101 5 mpk Day −3 vs. CD101 10 mpk Day −3 | NS | NS |
| CD101 5 mpk Day −3 vs. CD101 20 mpk Day −3 | NS | NS |
| CD101 5 mpk D −5 vs. CD101 10 mpk Day −5 | 0.0009 | 0.0015 |
| CD101 5 mpk D −5 vs. CD101 20 mpk Day −5 | 0.0009 | 0.0015 |
| CD101 5 mpk Day 0 vs. CD101 5 mpk Day −1 | NS | NS |
| CD101 5 mpk Day 0 vs. CD101 5 mpk Day −3 | NS | NS |
| CD101 5 mpk Day 0 vs. CD101 5 mpk Day −5 | NS | NS |
| CD101 5 mpk Day −1 vs. CD101 5 mpk Day −3 | NS | NS |
| CD101 5 mpk Day −1 vs. CD101 5 mpk Day −5 | NS | NS |
| CD101 5 mpk Day −3 vs. CD101 5 mpk Day −5 | 0.0183 | 0.0426 |
| CD101 10 mpk Day −1 vs. CD101 10 mpk Day −3 | NS | NS |
| CD101 10 mpk Day −1 vs. CD101 10 mpk Day −5 | NS | NS |
| CD101 10 mpk Day −3 vs. CD101 10 mpk Day −5 | NS | NS |
| CD101 20 mpk Day −1 vs. CD101 20 mpk Day −3 | 0.0387 | 0.033 |
| CD101 20 mpk Day −1 vs. CD101 20 mpk Day −5 | NS | NS |
| CD101 20 mpk Day −3 vs. CD101 20 mpk Day −5 | NS | NS |

NS—not significant

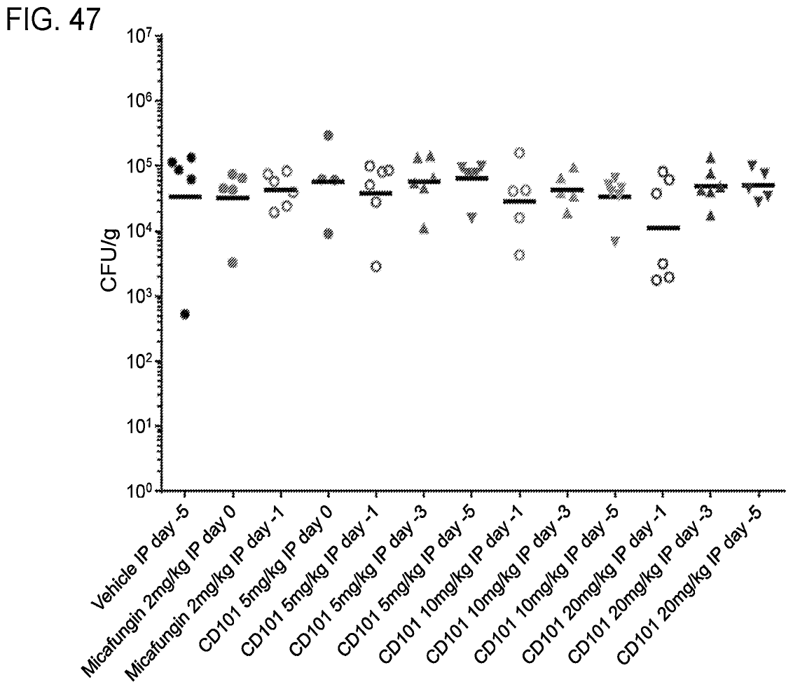

Lung burden. Terminal lung burden are shown in Table 18 and FIG. 47.

TABLE 18
Lung burden

| | Group | Standard | Log₁₀ Group | Log₁₀ reduction |
|---|---|---|---|---|
| | Geometric | Deviation | Geometric | from vehicle |
| Treatment | mean (CFU/g) | (CFU/g) | mean (CFU/g) | control (CFU/g) |
| Vehicle IP Day −5 | 5.93 × 10³ | 5.67 × 10⁴ | 3.77 | 0.00 |
| Micafungin 2 mg/kg IP Day 0 | 1.08 × 10⁴ | 3.10 × 10⁴ | 4.03 | −0.26 |
| Micafungin 2 mg/kg IP Day −1 | 4.34 × 10⁴ | 2.65 × 10⁴ | 4.64 | −0.86 |
| CD101 5 mg/kg IP Day 0 | 5.65 × 10⁴ | 1.28 × 10⁵ | 4.75 | −0.98 |
| CD101 5 mg/kg IP Day −1 | 3.75 × 10⁴ | 3.75 × 10⁴ | 4.57 | −0.80 |
| CD101 5 mg/kg IP Day −3 | 5.71 × 10⁴ | 5.21 × 10⁴ | 4.76 | −0.98 |
| CD101 5 mg/kg IP Day −5 | 6.36 × 10⁴ | 2.99 × 10⁴ | 4.80 | −1.03 |
| CD101 10 mg/kg IP Day −1 | 5.17 × 10³ | 5.89 × 10⁴ | 3.71 | 0.06 |
| CD101 10 mg/kg IP Day −3 | 7.34 × 10³ | 3.39 × 10⁴ | 3.87 | −0.09 |
| CD101 10 mg/kg IP Day −5 | 3.40 × 10⁴ | 1.97 × 10⁴ | 4.53 | −0.76 |
| CD101 20 mg/kg IP Day −1 | 1.12 × 10⁴ | 3.46 × 10⁴ | 4.05 | −0.28 |
| CD101 20 mg/kg IP Day −3 | 4.92 × 10⁴ | 4.12 × 10⁴ | 4.69 | −0.92 |
| CD101 20 mg/kg IP Day −5 | 8.35 × 10³ | 3.58 × 10⁴ | 3.92 | −0.15 |

### Conclusions

In this model of pulmonary aspergillosis, mice developed robust infection with ˜80% vehicle treated mice succumbing to the infection by Day 4 post infection and 100% mice by Day 5 post infection.

Single dose CD101 treatment one day pre-infection at 10 mg/kg or 20 mg/kg (the CD101 human dose (400 mg) AUC equivalent) resulted in statistically greater survival compared to comparator 2 mg/kg (the micafungin human dose (50 mg) AUC equivalent) micafungin treatment one day pre-infection.

### Example 10. CD101 Prophylactic Dose Rationale for Prevention of Aspergillus, Candida, and Pneumocystis Infections

Clinical pharmacokinetics of CD101 were compared to measures of nonclinical in vitro susceptibility and in vivo efficacy to guide dose selection for prevention of fungal infections.

### Methods

The protein binding of CD101 to mouse and human plasma proteins was measured by ultracentrifugation, where free compound is separated from protein-bound compound after 2.5 hr at 37° C. by sedimentation using high centrifugal force. Concentrations in plasma ranging from 7 to 60 μg/mL were tested and resulting samples were analyzed by LC-MS/MS.

The in vitro activity of CD101 was previously evaluated as part of the SENTRY international surveillance program using CLSI broth microdilution methodology (M38-A2, M27-A3; Pfaller, et al 2017). CD101 demonstrated potent activity against Aspergillus fumigatus (MEC₉₀=0.015 μg/mL) and Candida albicans (MIC₉₀=0.06 μg/mL) clinical isolates. These susceptibility data were then compared graphically to CD101 plasma concentrations from studies in healthy adults, adjusted for plasma protein binding. Nonclinical efficacy in a murine model of Pneumocystis pneumonia were also considered to evaluate CD101 doses for clinical investigation of antifungal prophylaxis.

### Results

In the protein binding study, across the concentrations tested, the percent of bound CD101 ranged from 96.4% to 98.0% with a mean of 97.4% in human plasma, whereas the percent of bound CD101 ranged from 99.2% to 99.3% with a mean of 99.2% in mouse plasma.

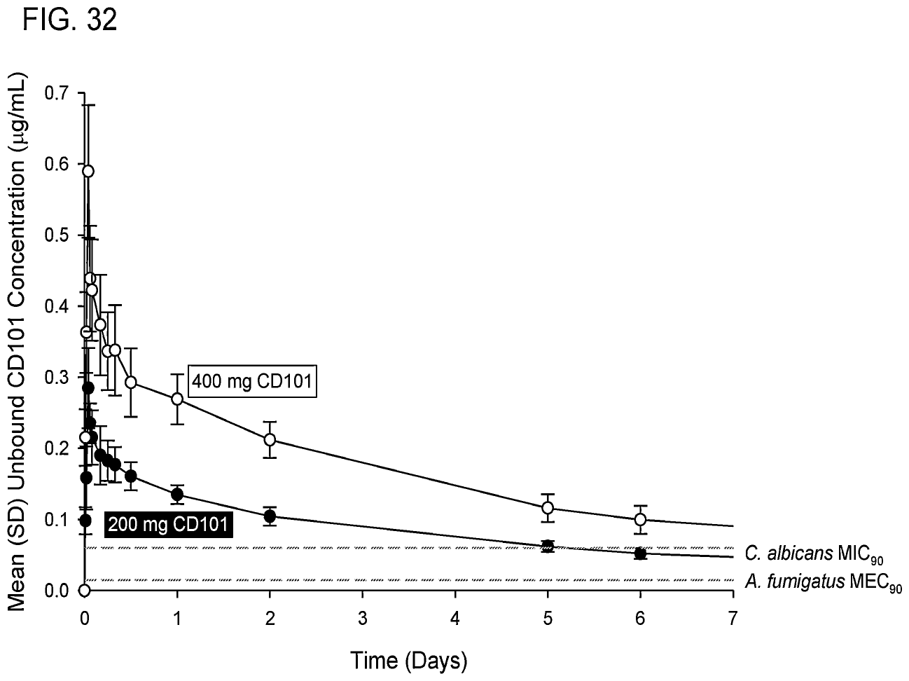

Mean unbound CD101 plasma concentrations in Phase 1 subjects following a single dose of 400 mg were above the MIC₉₀ for C. albicans for seven days, and CD101 plasma concentrations for both 400 mg and 200 mg were above the MEC₉₀ for A. fumigatus for 7 days (FIG. 32). Although standard in vitro MIC testing is not possible for Pneumocysis spp., CD101 prevented Pneumocysis pneumonia in mice at human equivalent doses of <50 mg, with results similar to standard of care (trimethoprim/sulfamethoxazole). A dose of 400 mg of CD101 appears sufficient for prevention of fungal infections, from the very first dose. Given accumulation of approximately 30% to 55% with repeat dose administration, and the fact that CD101 is fungicidal against Candida spp., CD101 at 200 mg may also be effective for fungal prophylaxis.

### Example 11. In Vitro Activity and In Vivo Tissue Distribution of CD101

### Methods

The in vitro activity of CD101 was evaluated against 153 A. fumigatus clinical isolates collected as part of the 2014 and 2015 JMI international SENTRY surveillance program. Susceptibility was determined by measuring the minimal effective concentration (MEC) values in accordance with CLSI broth microdilution guidelines (M38-A2). In vivo tissue distribution of CD101 in SD rats (N=3/time up to 5 d) after a 5 mg/kg IV CD101 dose. Plasma/tissue concentrations were measured by LC-MS/MS.

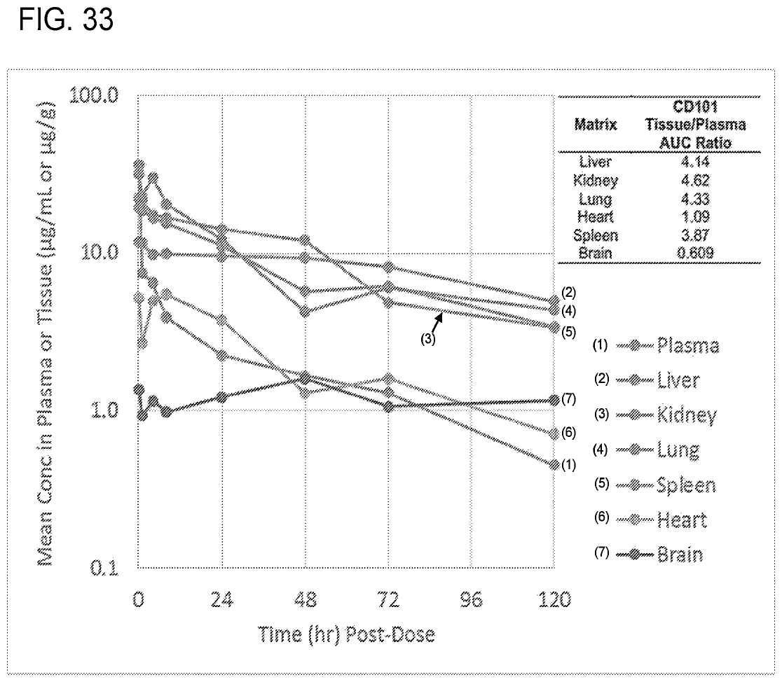

CD101 demonstrated potent in vitro activity against clinical A. fumigatus isolates with MEC50, MEC90 and MEC range values of 0.015, 0.015, and 50.0078-0.03 μg/mL, respectively. In vivo, CD101 tissue/plasma exposure ratios (˜4) were comparable among the major organs (liver, kidney, lung, spleen) suggesting efficient penetration. Also, longer tissue residence times were observed as t_{1/2} of CD101 in all tissues (40-77 h) studied were longer compared with plasma (39 h) (FIG. 33).

### Example 12. Pharmacodynamics of the Long Acting Echinocandin, CD101, in the Neutropenic Invasive Candidiasis Murine Model Using an Extended Interval Dosing Design

The current studies included pharmacokinetic/pharmacodynamic (PK/PD) evaluation of CD101 efficacy in a neutropenic murine model of disseminated candidiasis to assist with further clinical development of optimal dosing strategies. The studies were specifically designed to examine [1] pharmacokinetics of extended interval dosing in the murine model; [2] the CD101 dose-response relationships against a diverse group of strains including C. albicans, C. glabrata, and C. parapsilosis; [3] the PK/PD target exposures associated with efficacy against each species.

### Materials and Methods

Antifungal agent. CD101 dose solutions were prepared on the day of experimentation according to manufacturer instructions with 0.9% NaCl, 10% DMSO, and 1% Tween-20.

Strains. Ten clinical Candida strains were used for the in vivo treatment studies, including four C. albicans, three C. glabrata, and three C. parapsilosis strains (Table 18). This group was selected to encompass phenotypic variability in susceptibility to triazoles and echinocandins and based on similar fitness in the animal model as defined by the amount of growth in control animals over 24 h. The organisms were maintained, grown, and quantified on Sabouraud's dextrose agar (SDA) plates.

TABLE 19
Study organisms, CD101 susceptibility results, and comparative
susceptibility results to anidulafungin.

| | | CD101 | Anidulafungin |
|---|---|---|---|
| Organism | Strain | MIC (mg/L) | MIC (mg/L) |
| C. albicans | K-1 | 0.06 | 0.015 |
| | 580 | 0.06 | 0.015 |
| | 98-17 | 0.06 | 0.03 |
| | 98-210 | 0.03 | 0.015 |
| C. glabrata | 10956 | 1 | 1 |
| | 5592 | 0.125 | 0.06 |
| | 35315 | 0.5 | 0.25 |
| C. parapsilosis | 20519.069 | 1 | 4 |
| | 20477.048 | 1 | 2 |
| | 20423.072 | 0.5 | 1 |

In vitro susceptibility testing. All isolates were tested in accordance with the standards in CLSI document M27-A3. The MICs were determined visually after 24 h of incubation as the lowest concentration of drug that causes a significant diminution (≥50%) of growth compared to controls. MICs were determined on three separate occasions in duplicate. Results are expressed as the median of these results.

Animals. Six-week-old ICR Swiss/CD1 specific-pathogen-free female mice (Harlan Sprague-Dawley, Indianapolis, IN) weighing 23 to 27 g were used for all the studies.

Infection model. A neutropenic, murine, disseminated candidiasis model was used for the treatment studies. The mice were rendered neutropenic (polymorphonuclear cell count, <100/mm3) by injecting 150 mg/kg of cyclophosphamide (Mead Johnson Pharmaceuticals, Evansville, IN) subcutaneously 4 days before infection, 100 mg/kg of cyclophosphamide 1 day before infection, and additional cyclophosphamide doses (100 mg/kg) on Day 2 and Day 4 after infection to ensure neutropenia throughout the 168 h (7 d) study period. Three mice were included in each treatment and control group.

The organisms were subcultured on SDA plates 24 h prior to infection. The inoculum was prepared by placing 3 to 5 colonies into 5 ml of sterile pyrogen-free 0.15 M NaCl warmed to 35° C. The final inoculum was adjusted to a 0.6 transmittance at 530 nm. The fungal count of the inoculum determined by viable counts on SDA was 6.1±0.2 log₁₀ CFU/ml.

Disseminated infection with the Candida strains was achieved by injection of 0.1 ml of the inoculum via the lateral tail vein 2 h prior to the start of antifungal therapy. At the end of the study period, the animals were sacrificed by CO₂ asphyxiation. The kidneys of each mouse were aseptically removed and placed in 0.15 M NaCl at 4° C. The kidneys were homogenized and serially diluted 1:10, and the aliquots were plated onto SDA for viable fungal colony counts after incubation for 24 h at 35° C. The lower limit of detection was 100 CFU/ml. The results are expressed as the mean CFU/kidney for three mice.

Pharmacokinetics. Single-dose pharmacokinetic (PK) evaluation was undertaken following intraperitoneal (IP) doses of 1, 4, 16, and 64 mg/kg of CD101. Plasma from groups of three mice per time point (1, 3, 6, 12, 24, 48, and 72-h) was collected. The plasma drug concentrations were determined by liquid chromatography-tandem mass spectrometry. A noncompartmental model was used in the pharmacokinetic analysis. Elimination half-life was calculated by nonlinear least-squares technique. The area under the concentration-time curve (AUC) was calculated by the trapezoidal rule. Pharmacokinetic exposures for doses not directly measured in the PK study were estimated by linear extrapolation for higher and lower dose levels and by interpolation for dose levels within the dose range studied given the linear PK results. Protein binding was 99.2%.

Treatment efficacy and pharmacodynamic target determination of CD101. Neutropenic mice were infected with one of 10 Candida strains as described above. The dosing regimens were chosen to vary the magnitude of the 24-h AUC/MIC index and to attempt to produce treatment effects that ranged from no effect to a maximal effect. Five dose levels that varied from 0.25 to 64 mg/kg were administered once in a 0.2-ml volume by IP route for the 168 h study period. Due to enhanced effect against single isolate, additional studies at 0.0156 and 0.0625 mg/kg was examined for C. glabrata 5592. Groups of three mice were used for each dosing regimen and control group. At the end of the treatment period (168 h), the mice were euthanized, and their kidneys were immediately processed for CFU determination as described above.

Data analysis. A sigmoid dose-effect (Hill) model was used to measure the in vivo potency of CD101. The efficacy endpoints included the dose level required to produce a 24 h net static effect (no change in organism burden compared to that at the start of therapy) and the dose required to achieve a 1-log₁₀ reduction in colony counts (relative to the burden at the start of therapy), when achieved. The maximum response (Eₘₐₓ) was measured as the difference in the number of CFU/kidney relative to that of the untreated control animals. The doses associated with the stasis and 1-log₁₀ endpoint for each strain was calculated using the equation: log₁₀ D=[log₁₀ (E/(Eₘₐₓ−E))/N]+log₁₀ ED₅₀, where D is the drug dose, E is the control growth in untreated animals, Eₘₐₓ is the maximal effect, Nis the slope of the dose-response relationship, and ED₅₀ is the dose needed to achieve 50% of the maximal effect. The associated AUC/MIC targets were then calculated for each strain. We used the PK/PD index AUC/MIC in this study as this has been shown to be associated with treatment efficacy in previous in vivo studies with the echinocandins. The calculations were performed using both total and free drug concentrations. The coefficient of determination (R²) was used to estimate the variance that might be due to regression with the PK/PD index. Kruskal-Wallis one-way analysis of variance (ANOVA) was used to determine if the differences in PK/PD targets were significant between the species.

### Results

In vitro susceptibility studies. The MICs of CD101 for the selected strains is shown in Table 19. Additionally, given the similarity of CD101 to anidulafungin, the comparative MICs to anidulafungin are shown. Of note, the strains included those with known resistance (C. glabrata 10956 is echinocandin resistant secondary to FKS mutation FKS2_HS1_F659V) or reduced susceptibility (C. glabrata 35315) to echinocandins. Overall, the CD101 MIC varied by 32-fold for all strains.

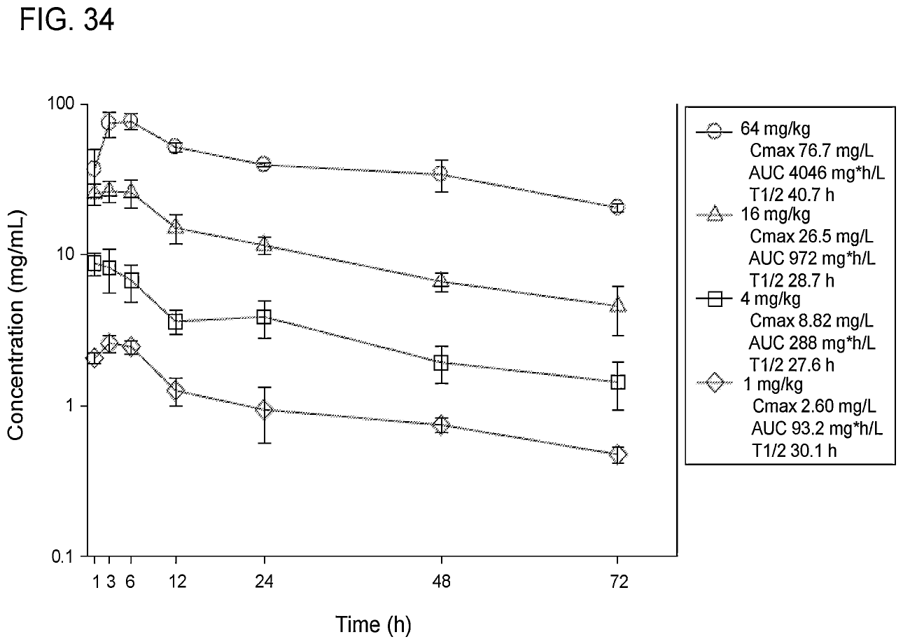

Pharmacokinetics. The time course of plasma concentrations of CD101 in mice after intraperitoneal doses of 1, 4, 16, and 64 mg/kg are shown in FIG. 34. Peak (Cₘₐₓ) levels ranged from 2.6-76.7 mg/L, AUC₀-93.2-40464 mg*h/L, and elimination half-life ranged from 28-41 h. The AUC₀-was linear (R²=1) over the dose range. Protein binding was 99.2%.

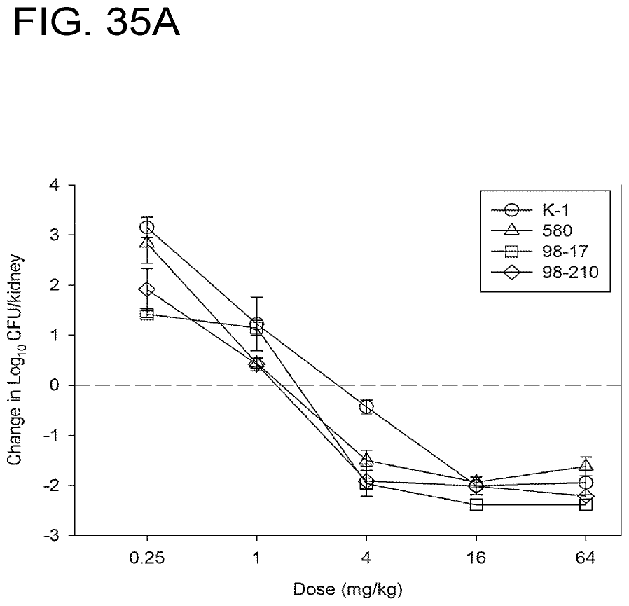

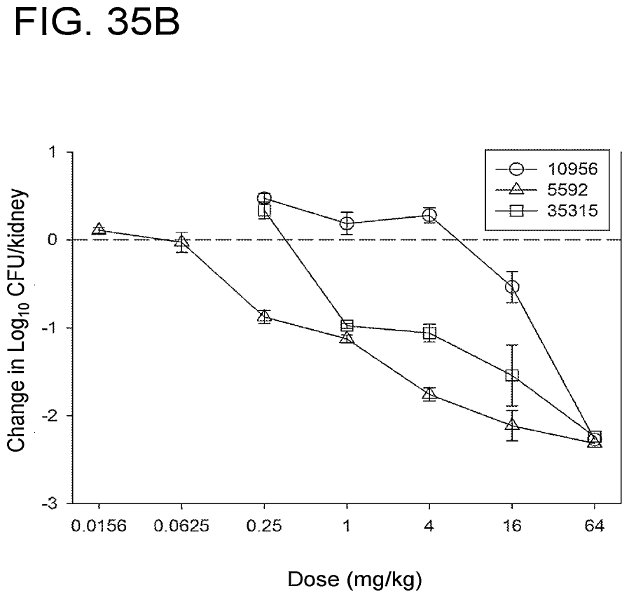

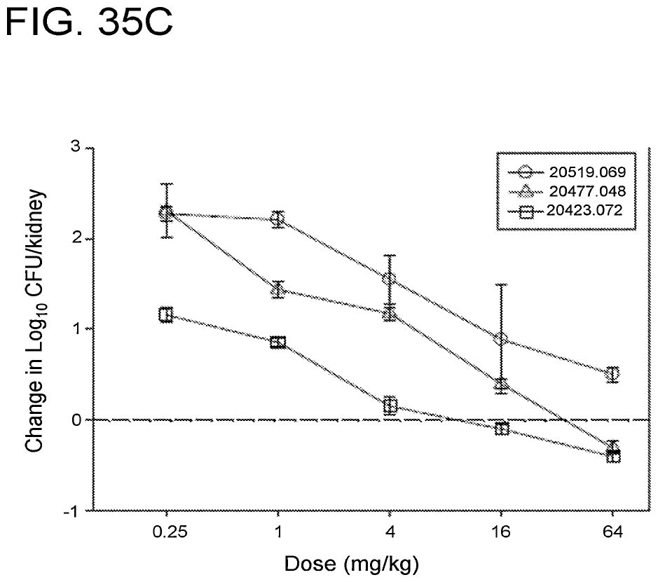

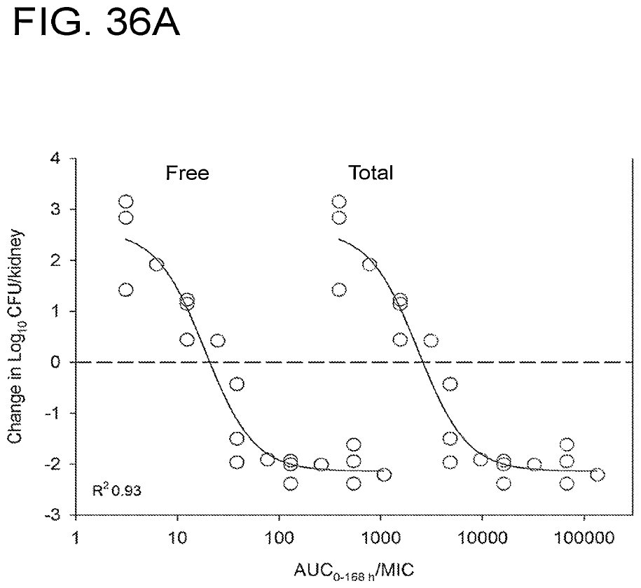

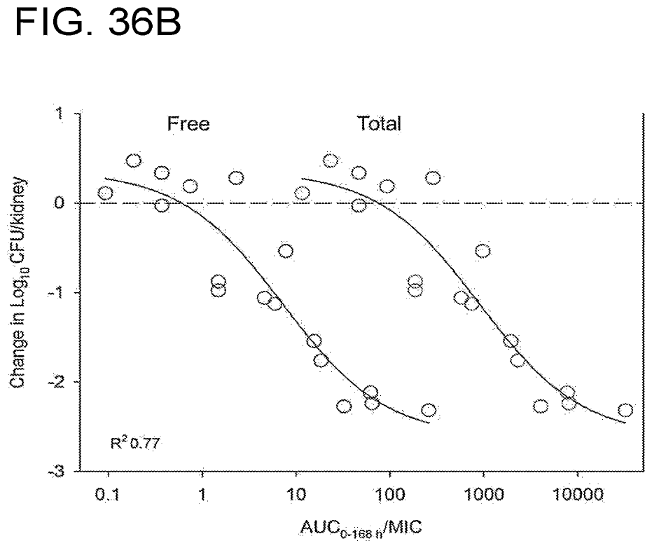

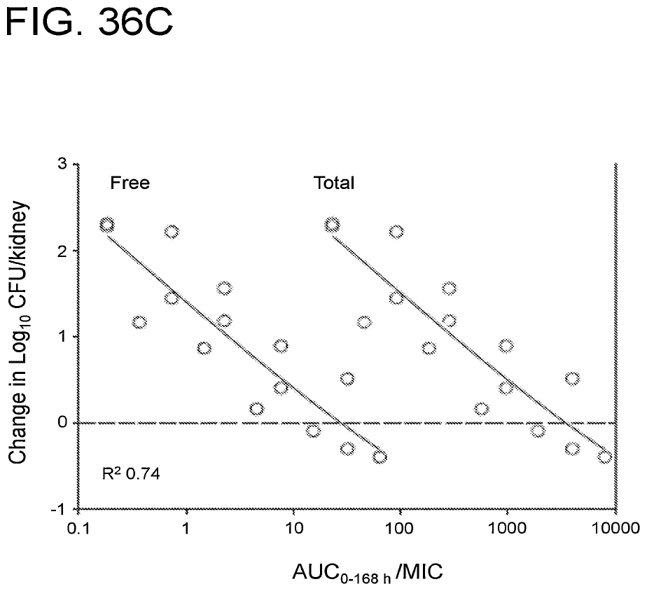

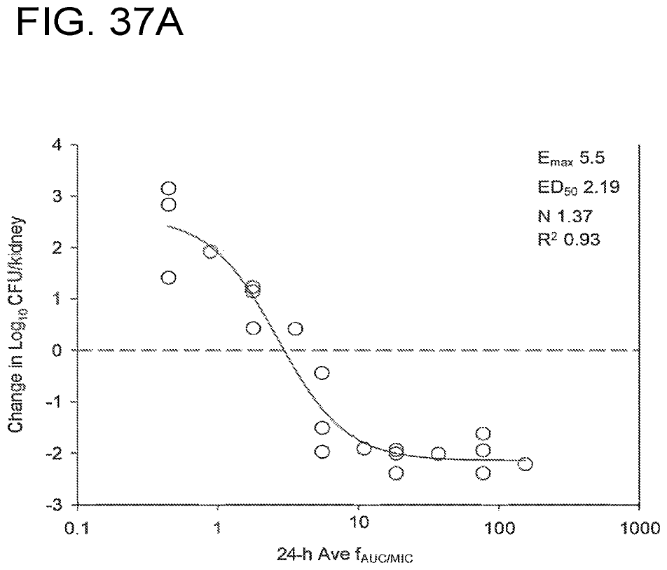

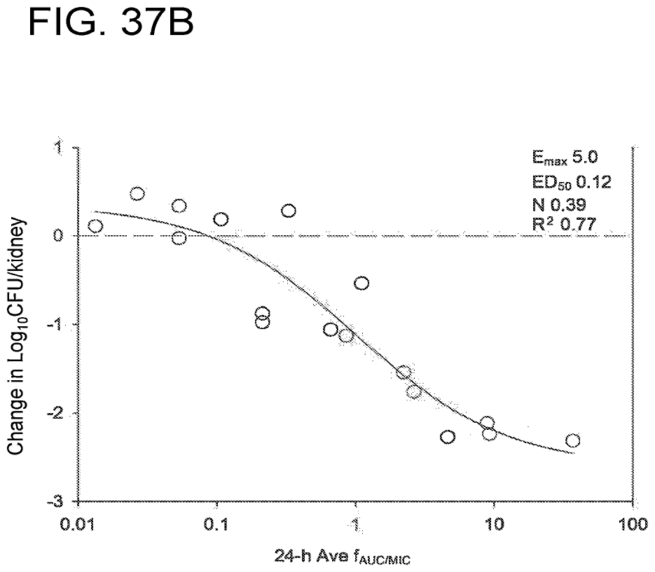

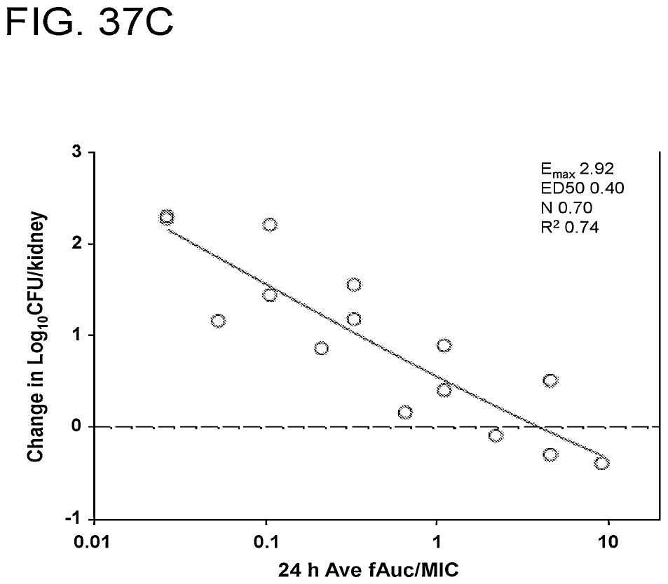

Treatment efficacy and pharmacodynamic target determination of CD101. At the start of therapy, mice had 4.2±0.2 log₁₀ CFU/kidney and burden increased in untreated controls to 7.2±0.6 log₁₀ CFU/kidney. The in vivo dose-response curves for each group of organisms is shown in FIGS. 35A-35C. Dose-dependent activity was observed with each group with marked potency at high doses against C. albicans and C. glabrata as a >2-log₁₀ kill was observed against a number of strains. Potency was less pronounced against C. parapsilosis, although based on the dose-response curve we speculate higher doses would have achieved similar activity for this species. The relationship between the PK/PD parameter AUC/MIC over the treatment period (168 h) and treatment effect is shown in FIGS. 36A-36C. Both free and total drug concentrations are shown with the best fit-line based on the Hill equation. The coefficients of determination (R²) were strong ranging from 0.74-0.93. Finally, shown in FIGS. 37A-37C is the average 24 h free drug AUC/MIC in order to augment comparison with previous echinocandin studies which have focused on 24 h PK/PD targets.

The doses necessary to achieve net stasis and 1-log₁₀ kill (when endpoint was achieved) are shown in Table 20. The corresponding total and free drug AUC/MIC values for the stasis and

1-log₁₀ kill endpoints are shown for the total experiment duration of 168 h (7 d). As above, also shown in the table is the average 24 h free drug AUC/MIC targets to allow for comparison to other echinocandin studies in this model. Stasis was achieved against all but a single strain and 1-log₁₀ kill was achieved against all C. albicans and C. glabrata but none of the C. parapsilosis strains. The median stasis free drug AUC₀₋₁₆₈/MIC targets for each organism group was: C. albicans 20.5, C. glabrata 0.5, and C. parapsilosis 18.2 (only two strains achieved the endpoint). The median stasis 24 h free drug AUC/MIC targets were: C. albicans 2.92, C. glabrata 0.07, and C. parapsilosis 2.61. The PK/PD targets for 1-log₁₀ kill endpoint were 2-4 fold higher than stasis targets indicating a relatively steep exposure-response relationship.

TABLE 20
Static and 1-log kill doses and associated AUC/MIC values in the neutropenic disseminated candidiasis model

| | | | | Stasis | Stasis | Stasis | | 1 log kill | 1 log kill | 1 log kill |
|---|---|---|---|---|---|---|---|---|---|---|
| | | | Static | total drug | free drug | 24 h Ave | 1 log kill | total drug | free drug | 24 h Ave |
| | | MIC | dose | AUC_{0-168 h}/ | AUC_{0-168 h}/ | free drug | dose | AUC_{0-168 h}/ | AUC_{0-168 h}/ | Free drug |
| Organism | Strain | (mg/L) | (mg/kg) | MIC | MIC | AUC/MIC | (mg/kg) | MIC | MIC | AUC/MIC |
| C. albicans | K-1 | 0.06 | 2.52 | 3197.16 | 25.58 | 3.65 | 5.26 | 6005.95 | 48.05 | 6.86 |
| | 580 | 0.06 | 1.20 | 1769.30 | 14.15 | 2.02 | 2.03 | 2667.21 | 21.34 | 3.05 |
| | 98-17 | 0.06 | 1.34 | 1918.43 | 15.35 | 2.19 | 2.73 | 3433.40 | 27.47 | 3.92 |
| | 98-210 | 0.03 | 1.06 | 3241.65 | 25.93 | 3.70 | 2.28 | 5875.95 | 47.01 | 6.72 |
| | Mean | | 1.53 | 2531.64 | 20.25 | 2.89 | 3.08 | 4495.63 | 35.97 | 5.14 |
| | Median | | 1.27 | 2557.79 | 20.46 | 2.92 | 2.51 | 4654.68 | 37.24 | 5.32 |
| | St Dev | | 0.67 | 796.71 | 6.37 | 0.91 | 1.49 | 1698.80 | 13.59 | 1.94 |
| C. glabrata | 10956 | 1 | 6.29 | 418.68 | 3.35 | 0.48 | 17.25 | 1052.22 | 8.42 | 1.20 |
| | 5592 | 0.125 | 0.06 | 43.16 | 0.35 | 0.05 | 0.43 | 317.50 | 2.54 | 0.36 |
| | 35315 | 0.5 | 0.34 | 62.50 | 0.50 | 0.07 | 2.39 | 367.06 | 2.94 | 0.42 |
| | Mean | | 2.23 | 174.78 | 1.40 | 0.20 | 6.69 | 578.93 | 4.63 | 0.66 |
| | Median | | 0.34 | 62.50 | 0.50 | 0.07 | 2.39 | 367.06 | 2.94 | 0.42 |
| | St Dev | | 3.52 | 211.44 | 1.69 | 0.24 | 9.20 | 410.63 | 3.29 | 0.47 |
| C. parapsilosis | 20519.069 | 1 | NA* | | | | NA | | | |
| | 20477.048 | 1 | 52.96 | 3339.42 | 26.72 | 3.82 | NA | | | |
| | 20423.072 | 0.5 | 9.62 | 1217.49 | 9.74 | 1.39 | NA | | | |

*NA, not achieved

### Discussion

In the current murine model pharmacodynamic study, we aimed to integrate the pharmacokinetic properties and in vitro potency to provide guidance on pharmacodynamic targets associated with efficacy against a clinically relevant and diverse group Candida spp. Indeed, the pharmacokinetics of CD101 were unique in that the elimination half-life in mice was prolonged (range 29-41 h). For comparison purposes, the half-life of other echinocandins in the same murine model are on average approximately 14 h. We also demonstrated promising in vitro potency against Candida spp. similar to previous larger surveillance antimicrobial susceptibility studies. Finally, we demonstrated CD101 has favorable in vivo efficacy using the murine disseminated candidiasis model with numerically lower PK/PD target exposures for most organisms compared to other echinocandins. For example, the median stasis 24 h free drug AUC/MIC against C. albicans was 2.92. This is 5- to 10-fold lower than caspofungin, micafungin, and anidulafungin targets against this species. An even larger difference was demonstrated for C. glabrata where CD101 free AUC/MIC targets were >10-fold lower than the three comparator echinocandins. C. parapsilosis PK/PD target analysis was limited in the current study as only two strains were evaluable for the stasis target endpoint, but here too CD101 free AUC/MIC targets were numerically lower than the three comparator echinocandins. It is important to note that due to the prolonged half-life, mice were protected from organism growth and disease for a neutropenic duration of 7 days. Taken together, the study demonstrates that CD101 is a potential valuable addition to the antifungal armamentarium given its unique pharmacokinetic properties and in vivo efficacy.

An important consideration in translating pre-clinical PK/PD target models to clinical medicine is to examine the targets identified in the context of human pharmacokinetics and surveillance susceptibility ranges. Pharmacokinetic study of CD101 in humans demonstrated a free drug AUC₀₋₁₆₈ of 30.2 mg*h/L for a 400 mg dose and 15.4 mg*h/L for a 200 mg dose. This would translate into an average 24 h AUC of approximately 4.3 and 2.2 mg*h/L, respectively, over a 7 day period. Thus, if a patient were to receive 400 mg of CD101 on Day 1 followed by a 200 mg on Day 8 to complete two weeks of therapy, the stasis target would be expected to be achieved against all C. albicans and C. parapsilosis isolates with MIC≤1 mg/L, and against all C. glabrata with MIC≤16 mg/L. The potency against C. glabrata, including an FKS mutant strain included in this study, deserves particular attention to further study given the rise of echinocandin resistance within this species. Overall, the data suggests CD101 exposures in humans would be expected to meet or exceed the stasis targets identified in this study for nearly all wild-type isolates for the examined species.

### Example 13. A. fumigatus (ATCC 13073) Disseminated Infection of Neutropenic ICR Mice: CD101 Prophylactic Efficacy

The study objective was to evaluate the efficacy of the test article, CD101, as prophylaxis in the Aspergillus fumigatus (ATCC 13073) disseminated infection model with neutropenic ICR mice.

### Methods

Inoculum Preparation. A. fumigatus (ATCC 13073) growth was taken from 96 hr potato dextrose agar (PDA) and re-suspended in 0.1% Tween 20. The culture was resuspended in 1 mL cold PBS (>1.0×10⁸ CFU/mL, OD620 2.3-2.8). The culture was then diluted in PBS to final cellular densities of 2.0×10⁵ CFU/mL. The actual colony counts were determined by plating dilutions on PDA plates to confirm inoculation concentration. The actual inoculum count was 1.85×10⁵ CFU/mL.

A. fumigatus (ATCC 13073) disseminated infection (IV). Groups of 6 female ICR mice weighing 22±2 g were used. Animals were immunosuppressed by three intraperitoneal (IP) injections of cyclophosphamide (the first at 6 mg/mouse 3 days before inoculation, the second and third at 2 mg/mouse on Day 1 then Day 4 after inoculation). On Day 0, animals were inoculated (0.1 mL/mouse) by intravenous (IV) injection into the tail vein with A. fumigatus (ATCC 13073), 1.85×104 CFU per mouse. CD101 at 5, 10 and 20 mg/kg as prophylaxis was administered subcutaneously (SC) once starting 5, 3 or 1 day before inoculation. In addition, CD101 at 3 mg/kg SC and the reference, amphotericin B, at 3 mg/kg by intraperitoneal (IP) injection were administered one hr after infection (See Table 21).

Mortality was observed for 14 days. A 50 percent or more (≥50%) increase in the survival rate compared to the vehicle control group indicates significant anti-infective activity. The health observations including body weight, hunched posture, ruffled fur, immobility and hypothermia were recorded daily for 14 days. Animals found moribund were to be humanely sacrificed with CO₂ asphyxiation in the study.

TABLE 21
Study design

| Group | Article | Route | Schedule | Day | mg/mL | mL/kg | mg/kg | Dose | (ICR) |
|---|---|---|---|---|---|---|---|---|---|
| 1 | Vehicle | — | None | — | — | — | — | — | 6 |
| 2 | AmpB | IP | Single | 0^{a} | 0.3 | 10 | 3 | 3 | 6 |
| 3 | CD101 | SC | Single | 0^{a} | 0.5 | 10 | 5 | 5 | 6 |
| 4 | CD101 | SC | Single | −5 | 0.5 | 10 | 5 | 5 | 6 |
| 5 | CD101 | SC | Single | −3 | 0.5 | 10 | 5 | 5 | 6 |
| 6 | CD101 | SC | Single | −1 | 0.5 | 10 | 5 | 5 | 6 |
| 7 | CD101 | SC | Single | −5 | 1 | 10 | 10 | 10 | 6 |
| 8 | CD101 | SC | Single | −3 | 1 | 10 | 10 | 10 | 6 |
| 9 | CD101 | SC | Single | −1 | 1 | 10 | 10 | 10 | 6 |
| 10 | CD101 | SC | Single | −5 | 2 | 10 | 20 | 20 | 6 |
| 11 | CD101 | SC | Single | −3 | 2 | 10 | 20 | 20 | 6 |
| 12 | CD101 | SC | Single | −1 | 2 | 10 | 20 | 20 | 6 |

### Results

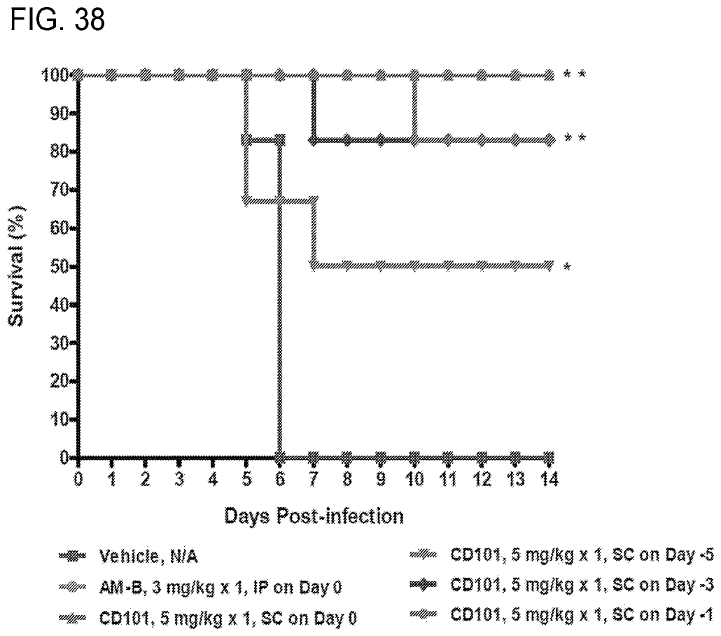

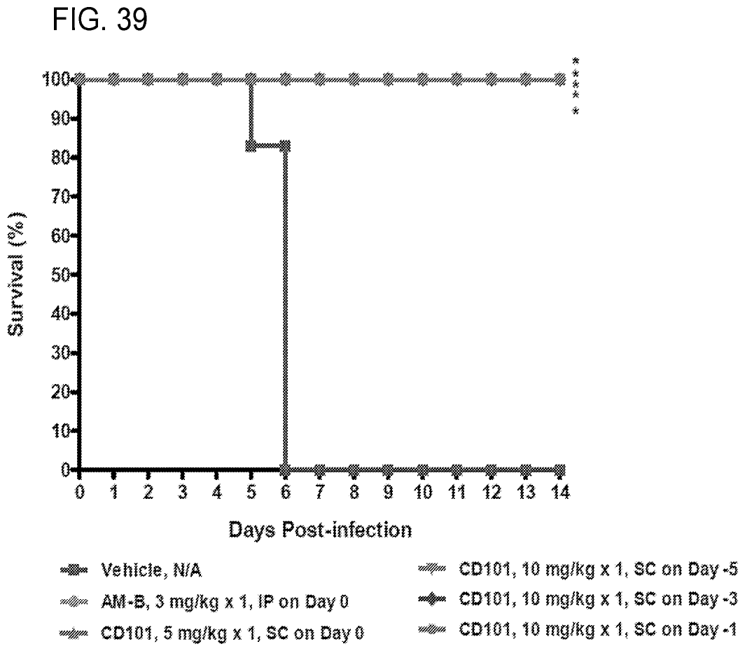

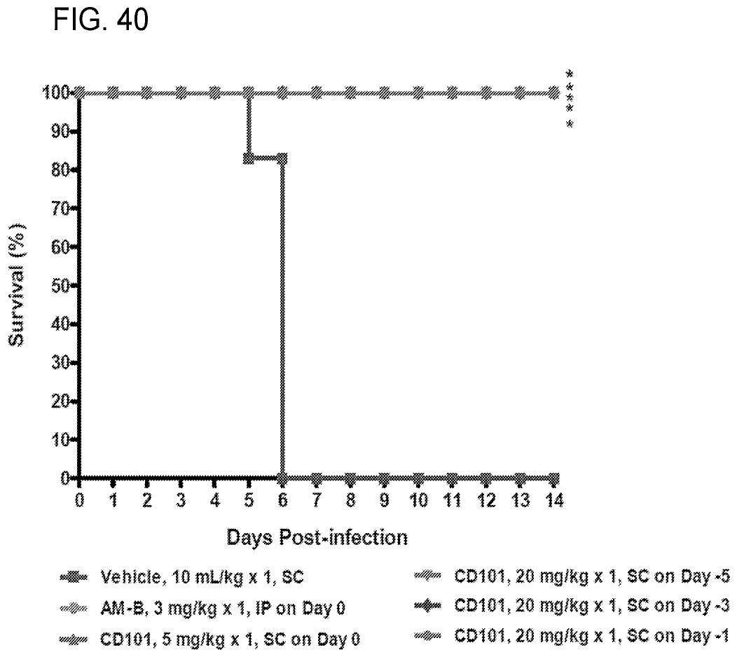

Subcutaneous administrations of CD101 at 5, 10, and 20 mg/kg on Day −5, −3 and −1 were associated with significant (≥50%) increase in the 14-day survival compared to the vehicle group (FIGS. 38-40). CD101 at 5 mg/kg SC and amphotericin B at 3 mg/kg IP administered 1 hr after infection were also associated with significant increase in the 14-day survival observation in the study.

In addition, the symptoms of infection including a decrease in the body weight, hunched posture, ruffled fur, immobility and hypothermia from were improved by subcutaneous administrations of CD101 at 5, 10 and 20 mg/kg on Day −5, −3 and −1 before infection (data not shown).

### Example 14. CD101 Tissue and Epithelial Lining Fluid Concentrations Substantiates its Use for Prophylaxis Treatment as Evident in Mouse Disseminated and Pulmonary Aspergillosis

CD101 has previously demonstrated robust efficacy in mouse antifungal models of aspergillosis. The distribution of CD101 into lung epithelial lining fluid (ELF) was studied to provide further substantiation of observed efficacy.

### Methods

CD101 (20 mg/kg) was administered by IP to 24 ICR mice. At pre-dose, 1, 3, 6, 12, 24, 48, and 72 hours post-dose, 3 mice/timepoint were anesthetized/euthanized for blood collection (plasma) and bronchoalveolar lavage fluid (BALF) collection with 2×0.5 mL flushes of saline. Urea levels for plasma/BALF normalization for the volume of lung epithelial lining fluid (ELF) calculation were quantified using a commercially-available spectrophotometry-based assay. CD101 concentrations in plasma/BALF samples were measured by LC with electrospray ionization tandem mass spectrometric (LC-MS/MS) detection.

Disseminated aspergillosis: ICR mice (6/grp) were made neutropenic by cyclophosphamide on Days −3 (270 mg/kg), +1 and +4 (90 mg/kg). IV infection with A. fumigatus ATCC 13073 (10⁴ CFU/mouse) on Day 0. Treatment (2 h after infection) with CD101 as a single dose (2 mg/kg IV and IP) or daily (0.5 mg/kg BID) dosing. Survival monitored for a 10 days. Same model was used for prophylaxis except CD101 was dosed on Days −1, −3 or −5.

Pulmonary aspergillosis: ICR mice (10/grp) were made neutropenic by cyclophosphamide on Day −4 (150 mg/kg), and CPM/cortisone on Day −1 (150/175 mg/kg). Intranasal infection with A. fumigatus AF293 (10⁵ CFU/mouse) on Day 0. Prophylaxis CD101 as a single dose (IP; 5, 10, 20 mg/kg) or posaconazole (PO; 2 and 10 mg/kg) 1 day prior to infection. Survival monitored for 10 days.

### Results

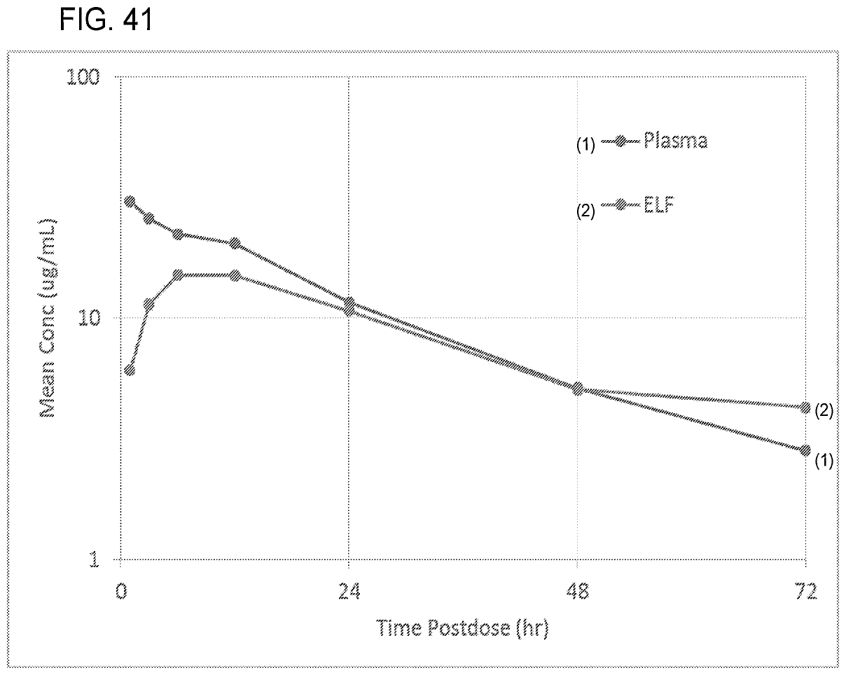

CD101 ELF concentrations reached a maximum by 4 hours and were comparable between plasma and ELF at 24 and 48 hours (FIG. 41). Concentration may potentially be higher in ELF by 72 hours suggesting possibly a longer half-life in ELF of 32 hour vs. 21 hour in plasma. Following CD101 administration, the mean maximum plasma concentration measured was 30.1 μg/mL and was observed at 1 hour post-dose, which was the first collection timepoint. The corresponding mean plasma AUC₀₋₇₂ and AUC_{inf} were 762 and 848 μg-hr/mL, respectively, with a half-life of 21.1 hours. The mean maximum ELF concentration measured was 15.1 μg/mL, which was reached at 6 hours post-dose. Corresponding mean ELF AUC₀₋₇₂ and AUC_{inf} were 606 and 802 μg-hr/mL, respectively, with a half-life of 31.9 hours. Based on AUC exposure ratios of ELF/plasma, the distribution of CD101 from plasma into lung ELF is close to unity (0.80 to 0.95).

For treatment of disseminated aspergillosis, CD101 by IV/IP at 0.2, 1, or 5 mg/kg BID×5 d showed a significant increase in survival compared to vehicle. Survival was comparable when given either a single 2 mg/kg or as 0.2 mg/kg BID×5 d dose. For prophylaxis, a single 5 mg/kg dose given up to 5 days prior to infection showed improved survival depending on day given. Doses 210 mg/kg showed 100% survival.

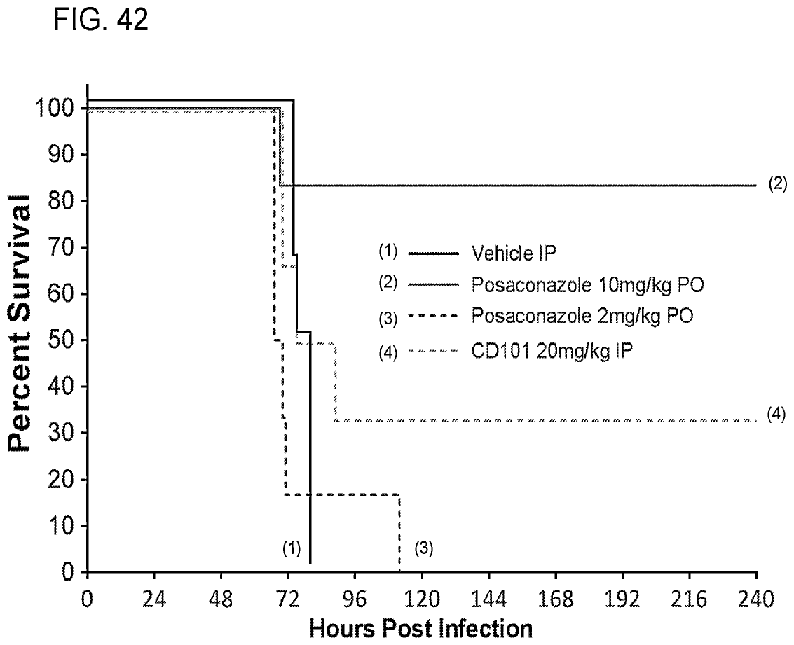

In the more challenging pulmonary aspergillosis model, dose-dependent increase in survival rate was observed from a single CD101 dose given one day prior to infection. The human (400 mg) AUC equivalent of 20 mg/kg in mice showed an increase in survival relative to control. Further comparison with posaconazole at the human AUC equivalent dose of 2 mg/kg (FIG. 42) suggests an advantage for CD101 with 30% survival rate compared to no survivors for posaconazole. Only posaconazole at 10 mg/kg showed a statistically-significant increase in survival rate relative to control.

### Example 15. High and Sustained CD101 Lung Epithelial Lining Fluid-to-Plasma Exposure Ratio from a Single Dose: Comparison to Posaconazole and Micafungin in a Mouse Pulmonary Aspergillosis Infection Model

CD101 has demonstrated in vitro potency and in vivo efficacy in mouse models of aspergillosis. The distribution of CD101 into lung ELF was studied to further substantiate this observed efficacy.

### Methods

Mice were dosed with CD101 (IP, 20 mg/kg) and then sacrificed for plasma and bronchoalveolar lavage fluid (BALF) collection between 0-72 hours. Urea for plasma/BALF normalization for ELF volume were quantified using spectrophotometry. CD101 concentrations in plasma/BALF samples were measured by LC-MS/MS. Total plasma concentrations were corrected for protein binding (99.2%).

Pulmonary aspergillosis: ICR mice (10/grp) were made neutropenic by cyclophosphamide on Day −4 (150 mg/kg), and cyclophosphamide/cortisone was given on Day −1 (150/175 mg/kg). Intranasal challenge with A. fumigatus AF293 (10⁵ CFU/mouse) was initiated on Day 0 and prophylaxis with CD101 as a single dose (IP; 5, 10, 20 mg/kg) or posaconazole (PO; 2 and 10 mg/kg) was started 1 day prior to infection. Survival was monitored for 10 days.

### Results

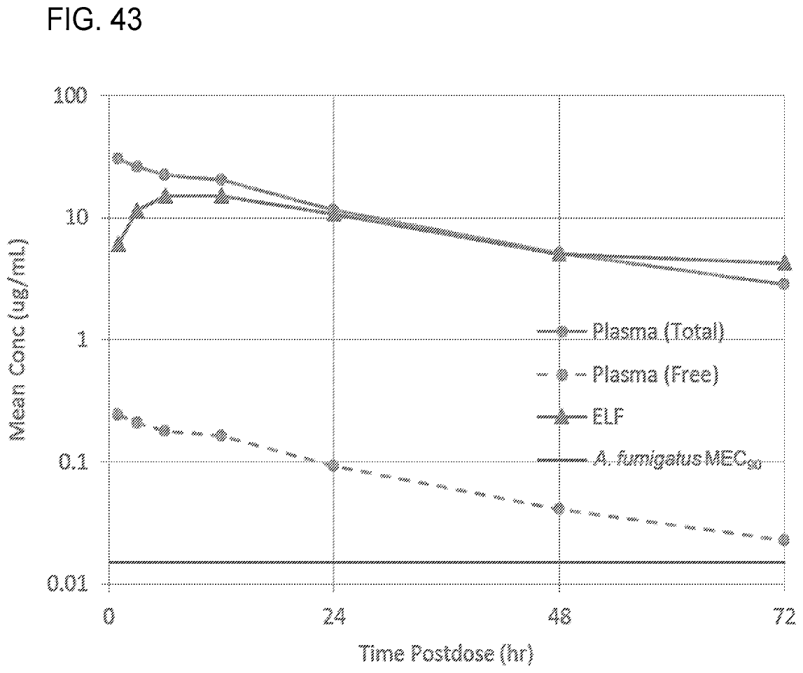

Maximum CD101 ELF concentrations were observed at 4 h and were comparable between plasma and ELF by 24 h post-dose as total-drug concentrations. At 72 h, mean ELF concentration (4 μg/mL) remained considerably higher than A. fumigatus MEC₉₀ of 0.015 μg/mL (FIG. 43). The resulting ELF/Plasma AUC ratio was 0.80 for total-drug and 100 for free-drug exposures, respectively.

Dose-dependent increase in survival was observed from a single prophylaxis CD101 dose. The human (400 mg) AUC equivalent of 20 mg/kg in mice showed an increase in survival relative to control. CD101 protein binding results show a higher free fraction (˜3×) in human vs mouse plasma suggesting a lower human dose may be equally protective. Further comparison with posaconazole at the human-equivalent dose of 2 mg/kg or micafungin human-equivalent dose (100 mg) of 5 mg/kg in mice suggests an advantage for CD101 with 30% survival rate compared to no survivors for posaconazole or micafungin. Only posaconazole at 10 mg/kg (5× higher than human AUC) showed a statistically-significant increase in survival rate relative to control.

### Example 16. Assessment of the Efficacy of CD101 and Comparators in a Murine Model of Pulmonary Aspergillosis

The overall aim of the studies was to assess the antifungal efficacy of CD101 by intraperitoneal administration in a murine model of pulmonary aspergillosis caused by Aspergillus fumigatus strain AF293 (A. fumigatus AF293) compared to comparators posaconazole and micafungin. The primary objective of the study was to compare survival between the treatment groups. The secondary objective was to compare lung burden in vehicle and test article treated animals.

### Methods

Animal strain and housing. Mice used in these studies were supplied by Charles River (Margate UK) and were specific pathogen free. The strain of mice used was ICR (also known as CD1 Mice) which is a well characterized outbred murine strain. Male mice were 11-15 g on receipt and allowed to acclimatize for at least 7 days.

Immunosuppression. Mice were immunosuppressed on Day −4 with 150 mg/kg cyclophosphamide administered intraperitoneally (IP), and on Day −1 with 150 mg/kg cyclophosphamide IP and 175 mg/kg cortisone acetate administered subcutaneously (SC). To prevent bacterial infection due to the immunosuppression mice were given once daily 50 mg/kg ceftazidime.

Preparation of Organism and Infection. A. fumigatus strain AF293 inoculum was prepared from spore cultures grown on Sabouraud Dextrose agar (SAB) containing 50 μg/mL chloramphenicol (SABC) in vented tissue culture flasks. Following incubation for 7-10 days at 30° C., spore cultures were washed in sterile phosphate buffered saline (PBS) containing 0.05% Tween 80. Spore count was determined using a haemocytometer and spores were diluted in PBS to ˜6.9×10⁶ CFU/mL. Inoculum concentration was confirmed by quantitative culture onto SABC agar.

Neutropenic mice lungs were infected with 0.04 mL (0.02 mL/nare) of ˜4.17×10⁶ CFU/mL (˜1.67×10⁵ CFU/mouse) of A. fumigatus AF293 by intranasal (IN) instillation under temporary 2.5% isoflurane induced anesthesia.

Preparation of Test Articles. Micafungin (Mycamine, Astellas) was provided as a 50 mg vial (Lot 02323002, expiry 08/2017) and was prepared as per manufacturer's instructions by adding 5 mL saline for injection (SFI) directly into the vial to make up a 10 mg/mL stock solution. This solution was then diluted further in SFI to a working concentration of 0.5 mg/mL. The compound was administered IP at 10 mL/kg to achieve a 2 mg/kg dose. It was prepared fresh once and stored at 4° C. until required.

Posaconazole (Noxafil 40 mg/mL oral suspension, Merck Sharp & Dohme Limited) was provided as a 40 mg/mL oral suspension (Lot N00801, expiry 04/2019). This suspension was then diluted further in water for infection (WFI) to a working concentration of 0.2 and 1 mg/mL. The suspension was administered orally (PO) at 10 mL/kg for 2 and 10 mg/kg doses respectively, was prepared fresh once and stored at 4° C. until required.

Vehicle and CD101 diluent was 10% DMSO/1% Tween 20 in SFI: 1 mL of Tween 20 was added to 10 mL DMSO, gently mixed and SFI added to a final volume of 100 mL. This was filter sterilised and maintained at room temperature before use for dosing or formulating CD101. The vehicle was administered IP at 10 mL/kg.

Test article CD101 stock was prepared at 6 mg/mL in 10% DMSO/1% Tween 20 diluent. A clear non particulate solution was obtained following gentle mixing. Study doses of 20 mg/kg (2 mg/mL) were prepared from the 6 mg/mL stock as required by diluting in 10% DMSO/1% Tween 20 diluent. The 6 mg/mL stock was used undiluted for the 60 mg/kg study dose. All doses were administered IP at 10 mL/kg. The study doses were kept at 4° C. until required.

Treatment. For this study, treatments were initiated on Day 1 pre infection according to treatment groups outlined in Table 22. A total of 36 mice (6/treatment group) were used in the study.

TABLE 22
Murine model of pulmonary aspergillosis treatment groups

| | Test | | | Dosing | Conc. | Dosage | | Total | Mice | End of |
|---|---|---|---|---|---|---|---|---|---|---|
| Group | Article | Route | Schedule | Day | mg/mL | ml/kg | mg/kg | Dose | (ICR) | study |
| 1 | Vehicle | IP | Single | −1 | — | — | — | — | 6 | 10 |
| 2 | Posaconazole | PO | Single | −1 | 1 | 10 | 10 | 2 | 6 | 10 |
| 3 | Posaconazole | PO | Single | −1 | 0.2 | 10 | 2 | 2 | 6 | 10 |
| 4 | Micafungin | IP | Single | −1 | 0.5 | 10 | 5 | 5 | 6 | 10 |
| 5 | CD101 | IP | Single | −1 | 2 | 10 | 20 | 20 | 6 | 10 |
| 6 | CD101 | IP | Single | −1 | 6 | 10 | 60 | 60 | 6 | 10 |

General health monitoring. The mice were monitored at a frequency appropriate for their clinical condition. Mouse weights were recorded at least once daily both to ensure animals remained within ethical limits and to monitor efficacy of treatment.

Endpoints. The primary endpoint for this study was survival within agreed ethical limits (>20% weight loss, severe hypothermia <34° C., inability to reach food or drink, severe hunching). Mice were monitored by daily weight measurements with observations as frequently as clinical condition required.

Mice presenting with severe clinical deterioration were humanely euthanized using an overdose of pentobarbitone administered by IP injection following clinical assessment and the time of death was recorded. Animal carcasses were stored at −20° C. before quantitative assessment of burden.

Ten days post infection all surviving animals were weighed and had their clinical condition assessed prior to being euthanized. Final survival numbers were recorded and analysed as described below and carcasses frozen at −20° C. prior to further processing.

Lung burden. A secondary endpoint for the study was terminal lung tissue burden. Immediately following confirmation of death, carcasses were frozen at −20° C. prior to tissue dissection and processing. The frozen carcasses were thawed at room temperature, the lungs removed and placed into pre-weighed bead-beating tubes containing 2 mL of PBS and subjected to mechanical disruption. Organ homogenates were diluted further in PBS and quantitatively cultured for A. fumigatus onto SABC and incubated at 30° C. for 24-48 hours.

In addition, a 300 μL aliquot of the undiluted lung tissue homogenate was stored at −80° C. for possible optional assessment of burden by qPCR.

Statistical analysis. Data were analysed using StatsDirect software (version 2.7.8):
1. Survival data were analysed using the Kaplan Meier and Log-Rank and Wilcoxon tests (using the Peto-Prentice weighting method).
2. Lung tissue burden data were analysed using the non-parametric Kruskal-Wallis test and if this was statistically significant all pairwise comparisons were analyzed (Conover-Inman).

### Results

The aim of this study was to determine the in vivo efficacy of CD101 and comparators in a murine model of pulmonary aspergillosis. The design of this study is summarized in Table 22. All treatments were well tolerated with no adverse signs observed.

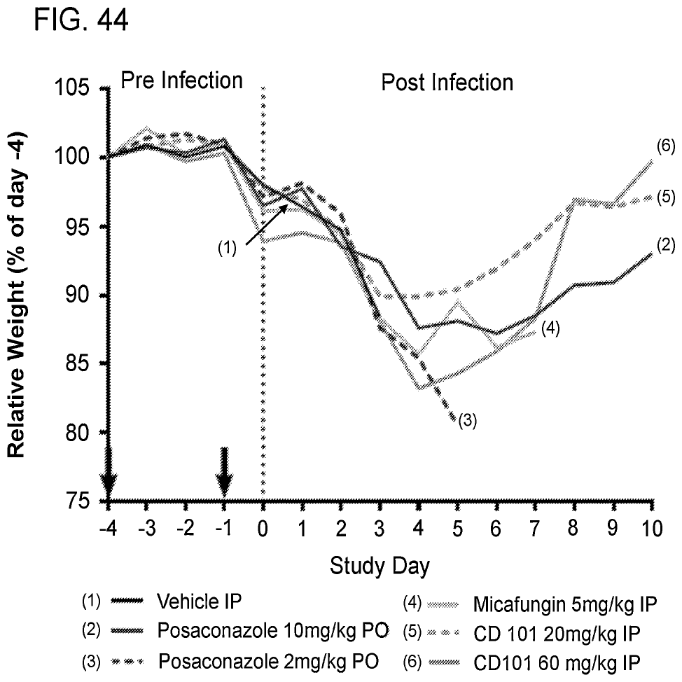

Body weights. Animal weights following infection with A. fumigatus AF293 are shown in FIG. 44. Animal weights are shown relative to the weight on Day 4 pre infection. Weights remained stable up to Day 1 pre-infection. Mice from all treatment groups lost weight following the immunosuppression on Day −1. The weight loss continued after the infection in almost all treatments groups up to Day 5 post infection; thereafter any mice that survived gained weight.

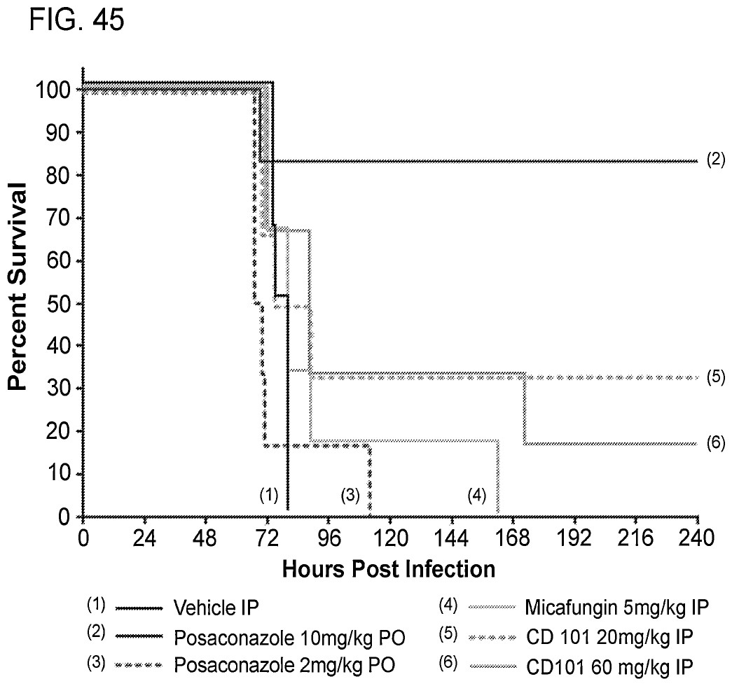

Survival. The median and mean survival for the various treatments are shown in Table 23, the survival plots in FIG. 45. Statistical outcomes from the Log-Rank and Wilcoxon test are shown in Table 24.

A robust survival model of pulmonary aspergillosis infection with A. fumigatus AF293 was established, with vehicle treated mice having a mean survival time of ˜77 h and a median survival time of ˜75 h post infection (range 74-80 h post infection). The study was terminated 10 days post infection as most mice had succumbed to the infection except in the 10 mg/kg posaconazole treatment group.

Treatment with test articles showed the following.
20 mg/kg CD101 dosed IP once on 1 Day pre-infection—Mice had a longer mean survival time of ˜130 h compared to the vehicle treated mice but a similar median survival time of ˜75 h post infection (range 70-240 h). However, this was not statistically better than vehicle treated mice (FIG. 45, Tables 24 and 25). Two mice survived to the end of the study.
60 mg/kg CD101 dosed IP once on 1 Day pre-infection—Mice had a longer mean and median survival time post infection compared to vehicle treated mice (˜123 h and ˜89 h respectively, range 73-240). A single mouse survived to the end of the study. Treatment with 60 mg/kg of CD101 did not result in significantly better survival compared to vehicle treated mice (FIG. 45, Tables 24 and 25).
Micafungin dosed at 5 mg/kg IP once on Day 1 pre-infection—Mice had a slightly longer mean and median survival time post infection compared to the vehicle treated mice (92 h and 80 h respectively, range 71-162 h) however, this was not statistically significant (FIG. 45, Tables 24 and 25).
Posaconazole dosed at 2 mg/kg PO once on Day 1 pre-infection—Mice had a similar mean survival time of 69 h and a median survival time of ˜78 h post infection (range 69-114 h). Statistically this was similar to the vehicle treated mice using the log-rank test but statistically lower survival compared to vehicle using the generalised Wilcoxon test (FIG. 45, Tables 24 and 25).
Posaconazole dosed at 10 mg/kg PO once on Day 1 pre-infection—Mice had a much longer mean survival time of 212 h post infection and median survival that could not be estimated as 5 mice survived to the end of the study (range 69-240 h). Statistically this was better than vehicle treated mice using the both the log-rank test and the generalized Wilcoxon test (FIG. 45, Tables 24 and 25).

TABLE 23
Mean and median survival per treatment group

| | Median | Mean |
|---|---|---|
| | Survival | Survival |
| Treatment | (Hours) | (Hours) |
| Vehicle IP | 75.3 | 77.4 |
| Posaconazole 10 mg/kg PO | cannot estimate | 211.5 |
| Posaconazole 2 mg/kg PO | 69.0 | 77.5 |
| Micafungin 5 mg/kg IP | 80.0 | 92.0 |
| CD101 20 mg/kg IP | 74.7 | 130.4 |
| CD101 60 mg/kg IP | 88.7 | 122.7 |

TABLE 24
Log-Rank and Wilcoxon test output for different comparisons

| | | Wilcoxon |
|---|---|---|
| Comparison | Log-Rank | (Peto- Prentice) |
| Vehicle vs. Posaconazole 10 mg/kg | P = 0.0182 | P = 0.0441 |
| Vehicle vs. Posaconazole 2 mg/kg | NS | P = 0.0391 |
| Vehicle vs. Micafungin 5 mg/kg | NS | NS |
| Vehicle vs. CD101 20 mg/kg | NS | NS |
| Vehicle vs. CD101 60 mg/kg | NS | NS |

NS—not significant

A small satellite study looking at the effect of immunosuppression (n=6 mice) was running with one week delay and a different batch of mice. Two mice in the study were lost several days after the Day −1 combination immunosuppression (cyclophosphamide and cortisone acetate), the remaining four mice in the study survived to the end of the study. The loss of the two mice was most likely due to secondary infection due to Pseudomonas aeruginosa, the source of which is not clear.

The main study data are unlikely to be affected by secondary infections as the positive control included, posaconazole, showed good efficacy against the infection in line with expectations based on previous results at EVOTEC.

Lung Burden

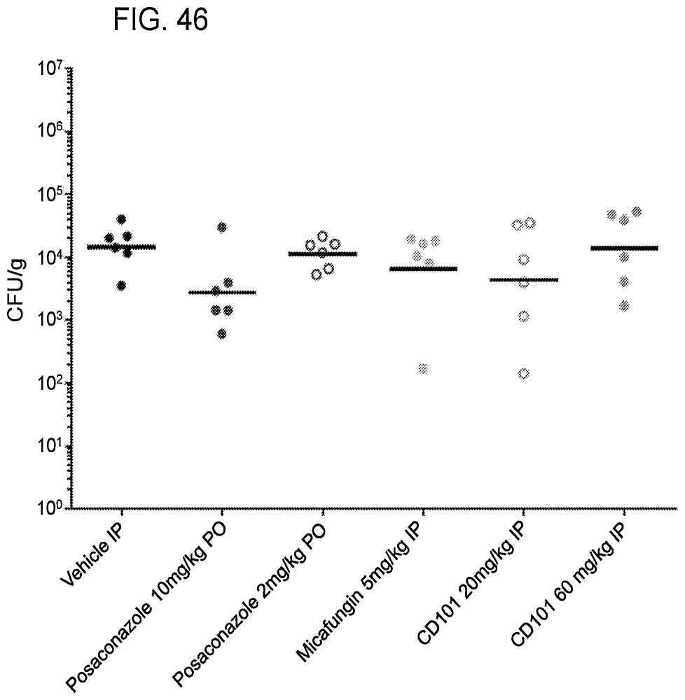

Terminal lung burdens are shown in Table 25 and FIG. 46.

TABLE 25
Lung burden

| | Group | | | |
|---|---|---|---|---|
| | Geometric | Standard | Log₁₀ Group | Log₁₀ reduction |
| | mean | Deviation | Geometric | from vehicle |
| Treatment | (CFU/g) | (CFU/g) | mean (CFU/g) | control (CFU/g) |
| Vehicle IP Day −1 | 1.48 × 10⁴ | 1.24 × 10⁴ | 4.17 | 0.00 |
| Posaconazole 10 mg/kg PO Day −1 | 2.75 × 10³ | 1.15 × 10⁴ | 3.44 | 0.73 |
| Posaconazole 2 mg/kg PO Day −1 | 1.14 × 10⁴ | 6.17 × 10³ | 4.06 | 0.11 |
| Micafungin 5 mg/kg IP Day −1 | 6.61 × 10³ | 7.34 × 10³ | 3.82 | 0.35 |
| CD101 20 mg/kg IP Day −1 | 4.37 × 10³ | 1.60 × 10⁴ | 3.64 | 0.53 |
| CD101 60 mg/kg IP Day −1 | 1.38 × 10⁴ | 2.30 × 10⁴ | 4.14 | 0.03 |

### Conclusion

In this model of pulmonary aspergillosis, mice developed a robust infection with vehicle treated mice succumbing to the infection by Day 4 post infection. CD101 administered at 20 and 60 mg/kg once one day pre-infection resulted in slight increase in survival, which was statistically longer than the vehicle treatment. The comparator micafungin dosed at 5 mg/kg once one day pre-infection did not show any improvement in survival, with all mice succumbing to the infection by Day 7 post infection. The comparator posaconazole dosed at 2 mg/kg once one day pre-infection did not show any improvement in survival compared to the vehicle mice, with all mice succumbing to the infection by Day 6 post infection. Increasing the dose of posaconazole to 10 mg/kg and dosed once 1 day pre-infection resulted in >80% mice surviving to the end of the study, significantly longer than the vehicle control treatment.

### OTHER EMBODIMENTS

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present invention that come within known or customary practice within the art to which the invention pertains and may be applied to the essential features hereinbefore set forth, and follows in the scope of the claims.

Other embodiments are within the claims.

1. A method of reducing the likelihood of a fungal infection in a subject comprising administering to the subject a pharmaceutical composition comprising CD101 salt, or a neutral form thereof, and one or more pharmaceutically acceptable excipients, wherein the pharmaceutical composition is administered in an amount and for a duration sufficient to reduce the likelihood of the fungal infection.

2. The method of claim 1, wherein the pharmaceutical composition comprises from 50 mg to 1200 mg of the CD101 salt, or a neutral form thereof.

3. The method of claim 1, wherein:
the subject is immunocompromised;
the subject has a cancer, an autoimmune disorder, or HIV/AIDs; or
the subject is a patient admitted to a hospital.

4. The method of claim 1, wherein the likelihood of a fungal infection in the subject is associated with one or more risk factors selected from:
an immunosuppressive condition, an immunosuppressive treatment, or a combination thereof;
a diagnostic or therapeutic procedure;
an injury;
an age-related risk factor; or
an environmental risk factor.

5. The method of claim 4, wherein:
the immunosuppressive treatment is a chemotherapy, a radiation therapy, a corticosteroid treatment, an anti-TNF therapy, an immunosuppressive drug, or a combination thereof;
the immunosuppressive condition is associated with a humoral immune deficiency, T cell deficiency, leukopenia, neutropenia, asplenia, complement deficiency, or a combination thereof.
the diagnostic or therapeutic procedure is a biopsy, an endoscopy, a catheterization, an intubation, a ventilation, a surgery, an implantation, a transplantation, or a combination thereof;
the injury to the skin or mucous membranes;
the age-related risk factor is an age greater than or equal to 65 years;
the age-related risk factor is an age less than or equal to 31 days; or
the environmental risk factor is environmental contamination by an airborne fungus.

6. The method of claim 1, wherein the CD101 salt is CD101 acetate.
